SEQUENCE LISTING (167 sequences)

SEQ ID NO: 1 - protein, 559 aa, synthetic construct
  REGION 1..559: note = RD114TR Fusion Protein
MKLPTGMVILCSLIIVRAGFDDPRKAIALVQKQHGKPCECSGGQVSEAPPNSIQQVTCPGKTAYLMTNQKWKCRVTPKISPSGGELQNCPCNTFQDSMHSSCYTEYRQCRRINKTYYTATLLKIRSGSLNEVQILQNPNQLLQSPCRGSINQPVCWSATAPIHISDGGGPLDTKRVWTVQKRLEQIHKAMTPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTRLCVQASLLPDIDINPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLASNPLWTGLQGFLPYLLPLLGPLLTLLLILTIGPCVFNRLVQFVKDRISVVQALVLTQQYHQLKPL

SEQ ID NO: 2 - protein, 662 aa, synthetic construct
  REGION 1..662: note = Feline Endogeneous Virus
MKPPAGMVFLWVLTSLGAGIGAKIVKEGNPHQVYTLTWQIYSQSGEVVWEVQGNHALNTWWPPLTPDFCQLAAGLDTWDIPARSPKNLQSYMGERIQQMTAHGCSSPTARCRLAQAEFYVCPRDNRDRATAHRCGGYEEYFCSAWGCETTGDAYWQPTSSWDLITITRGYTKPDPDGHTCYYKKGTEGYHHWISPLSLPLKITFTDSGKRALGWQTGYTWGLRWYLPGKDRGIVLKIKLKIDTITQTVGPNLVLADQKAPVQLAIPVQPPRAPTQTPGINPVNSTLSPSLGYPTPPLDRAQGDRLLNLVQGVYLTLNLTAPNQTQDCWLCLTAKPPYYQGVAIIGNFTNHTNAPLRCSTTPRHGLTLTEVTGHGLCIGKIPPSHQNLCSQTIPSVGQGPYYLTAPNGTYWVCNTGLTPCISLQVLNNTADYCILIELWPKIFYHDSEYIYGHYEPGGRFRREPVSLTVALLLGGLTMGSLAAGIGTGTAALIETNQFKQLQIAMHSDIQALEESISALERSLTSLSEVVLQNRRGLDLLFLQEGGLCAALKEECCFYADHTGIVRDSMAKLRERLKQRQKLFESQQGWFEGWYNKSPWFTTLVSSLMGPLILLLLILMFGPCILNRLVQFIRERLSVIQALVLTQQYHQLRQFDAERPDTIE

SEQ ID NO: 3 - protein, 511 aa, synthetic construct
  REGION 1..511: note = Vesicular Stomatitis Virus
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 4 - protein, 685 aa, synthetic construct
  REGION 1..685: note = Gibbon ape leukemia virus
MVLLPGSMLLTSNLHHLRHQMSPGSWKRLIILLSCVFGGGGTSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKREAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKILVLRQKYQALENEGNL

SEQ ID NO: 5 - protein, 565 aa, synthetic construct
  REGION 1..565: note = RD114TR fusion protein
MKLPTGMVILCSLIIVRAGFDDPRKAIALVQKQHGKPCECSGGQVSEAPPNSIQQVTCPGKTAYLMTNQKWKCRVTPKNLTPSGGELQNCPCNTFQDSMHSSCYTEYRQCRANNKTYYTATLLKIRSGSLNEVQILQNPNQLLQSPCRGSINQPVCWSATAPIHISDGGGPLDTKRVWTVQKRLEQIHKAMHPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTGLCVQASLLPDIDIIPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLASNPLWTGLQGFLPYLLPLLGPLLTLLLILTIGPCVFSRLMAFINDRLNVVHAMVLAQQYQALKAEEEAQD

SEQ ID NO: 6 - protein, 10 aa, Homo sapiens
YLYDSETKNA

SEQ ID NO: 7 - protein, 9 aa, Homo sapiens
HLMDQPLSV

SEQ ID NO: 8 - protein, 9 aa, Homo sapiens
GLLKKINSV

SEQ ID NO: 9 - protein, 9 aa, Homo sapiens
FLVDGSSAL

SEQ ID NO: 10 - protein, 10 aa, Homo sapiens
FLFDGSANLV

SEQ ID NO: 11 - protein, 9 aa, Homo sapiens
FLYKIIDEL

SEQ ID NO: 12 - protein, 11 aa, Homo sapiens
FILDSAETTTL

SEQ ID NO: 13 - protein, 9 aa, Homo sapiens
SVDVSPPKV

SEQ ID NO: 14 - protein, 8 aa, Homo sapiens
VADKIHSV

SEQ ID NO: 15 - protein, 9 aa, Homo sapiens
IVDDLTINL

SEQ ID NO: 16 - protein, 9 aa, Homo sapiens
GLLEELVTV

SEQ ID NO: 17 - protein, 10 aa, Homo sapiens
TLDGAAVNQV

SEQ ID NO: 18 - protein, 10 aa, Homo sapiens
SVLEKEIYSI

SEQ ID NO: 19 - protein, 9 aa, Homo sapiens
LLDPKTIFL

SEQ ID NO: 20 - protein, 9 aa, Homo sapiens
YTFSGDVQL

SEQ ID NO: 21 - protein, 9 aa, Homo sapiens
YLMDDFSSL

SEQ ID NO: 22 - protein, 9 aa, Homo sapiens
KVWSDVTPL

SEQ ID NO: 23 - protein, 11 aa, Homo sapiens
LLWGHPRVALA

SEQ ID NO: 24 - protein, 11 aa, Homo sapiens
KIWEELSVLEV

SEQ ID NO: 25 - protein, 9 aa, Homo sapiens
LLIPFTIFM

SEQ ID NO: 26 - protein, 9 aa, Homo sapiens
FLIENLLAA

SEQ ID NO: 27 - protein, 11 aa, Homo sapiens
LLWGHPRVALA

SEQ ID NO: 28 - protein, 9 aa, Homo sapiens
FLLEREQLL

SEQ ID NO: 29 - protein, 9 aa, Homo sapiens
SLAETIFIV

SEQ ID NO: 30 - protein, 9 aa, Homo sapiens
TLLEGISRA

SEQ ID NO: 31 - protein, 9 aa, Homo sapiens
ILQDGQFLV

SEQ ID NO: 32 - protein, 10 aa, Homo sapiens
VIFEGEPMYL

SEQ ID NO: 33 - protein, 9 aa, Homo sapiens
SLFESLEYL

SEQ ID NO: 34 - protein, 9 aa, Homo sapiens
SLLNQPKAV

SEQ ID NO: 35 - protein, 9 aa, Homo sapiens
GLAEFQENV

SEQ ID NO: 36 - protein, 9 aa, Homo sapiens
KLLAVIHEL

SEQ ID NO: 37 - protein, 9 aa, Homo sapiens
TLHDQVHLL

SEQ ID NO: 38 - protein, 11 aa, Homo sapiens
TLYNPERTITV

SEQ ID NO: 39 - protein, 9 aa, Homo sapiens
KLQEKIQEL

SEQ ID NO: 40 - protein, 10 aa, Homo sapiens
SVLEKEIYSI

SEQ ID NO: 41 - protein, 11 aa, Homo sapiens
RVIDDSLVVGV

SEQ ID NO: 42 - protein, 9 aa, Homo sapiens
VLFGELPAL

SEQ ID NO: 43 - protein, 9 aa, Homo sapiens
GLVDIMVHL

SEQ ID NO: 44 - protein, 9 aa, Homo sapiens
FLNAIETAL

SEQ ID NO: 45 - protein, 9 aa, Homo sapiens
ALLQALMEL

SEQ ID NO: 46 - protein, 9 aa, Homo sapiens
ALSSSQAEV

SEQ ID NO: 47 - protein, 11 aa, Homo sapiens
SLITGQDLLSV

SEQ ID NO: 48 - protein, 9 aa, Homo sapiens
QLIEKNWLL

SEQ ID NO: 49 - protein, 9 aa, Homo sapiens
LLDPKTIFL

SEQ ID NO: 50 - protein, 9 aa, Homo sapiens
RLHDENILL

SEQ ID NO: 51 - protein, 9 aa, Homo sapiens
YTFSGDVQL

SEQ ID NO: 52 - protein, 9 aa, Homo sapiens
GLPSATTTV

SEQ ID NO: 53 - protein, 11 aa, Homo sapiens
GLLPSAESIKL

SEQ ID NO: 54 - protein, 9 aa, Homo sapiens
KTASINQNV

SEQ ID NO: 55 - protein, 9 aa, Homo sapiens
SLLQHLIGL

SEQ ID NO: 56 - protein, 9 aa, Homo sapiens
YLMDDFSSL

SEQ ID NO: 57 - protein, 9 aa, Homo sapiens
LMYPYIYHV

SEQ ID NO: 58 - protein, 9 aa, Homo sapiens
KVWSDVTPL

SEQ ID NO: 59 - protein, 11 aa, Homo sapiens
LLWGHPRVALA

SEQ ID NO: 60 - protein, 9 aa, Homo sapiens
VLDGKVAVV

SEQ ID NO: 61 - protein, 9 aa, Homo sapiens
GLLGKVTSV

SEQ ID NO: 62 - protein, 9 aa, Homo sapiens
KMISAIPTL

SEQ ID NO: 63 - protein, 11 aa, Homo sapiens
GLLETTGLLAT

SEQ ID NO: 64 - protein, 9 aa, Homo sapiens
TLNTLDINL

SEQ ID NO: 65 - protein, 9 aa, Homo sapiens
VIIKGLEEI

SEQ ID NO: 66 - protein, 9 aa, Homo sapiens
YLEDGFAYV

SEQ ID NO: 67 - protein, 11 aa, Homo sapiens
KIWEELSVLEV

SEQ ID NO: 68 - protein, 9 aa, Homo sapiens
LLIPFTIFM

SEQ ID NO: 69 - protein, 10 aa, Homo sapiens
ISLDEVAVSL

SEQ ID NO: 70 - protein, 10 aa, Homo sapiens
KISDFGLATV

SEQ ID NO: 71 - protein, 11 aa, Homo sapiens
KLIGNIHGNEV

SEQ ID NO: 72 - protein, 9 aa, Homo sapiens
ILLSVLHQL

SEQ ID NO: 73 - protein, 9 aa, Homo sapiens
LDSEALLTL

SEQ ID NO: 74 - protein, 13 aa, Homo sapiens
VLQENSSDYQSNL

SEQ ID NO: 75 - protein, 11 aa, Homo sapiens
HLLGEGAFAQV

SEQ ID NO: 76 - protein, 9 aa, Homo sapiens
SLVENIHVL

SEQ ID NO: 77 - protein, 9 aa, Homo sapiens
YTFSGDVQL

SEQ ID NO: 78 - protein, 9 aa, Homo sapiens
SLSEKSPEV

SEQ ID NO: 79 - protein, 10 aa, Homo sapiens
AMFPDTIPRV

SEQ ID NO: 80 - protein, 9 aa, Homo sapiens
FLIENLLAA

SEQ ID NO: 81 - protein, 9 aa, Homo sapiens
FTAEFLEKV

SEQ ID NO: 82 - protein, 9 aa, Homo sapiens
ALYGNVQQV

SEQ ID NO: 83 - protein, 9 aa, Homo sapiens
LFQSRIAGV

SEQ ID NO: 84 - protein, 11 aa, Homo sapiens
ILAEEPIYIRV

SEQ ID NO: 85 - protein, 9 aa, Homo sapiens
FLLEREQLL

SEQ ID NO: 86 - protein, 10 aa, Homo sapiens
LLLPLELSLA

SEQ ID NO: 87 - protein, 9 aa, Homo sapiens
SLAETIFIV

SEQ ID NO: 88 - protein, 11 aa, Homo sapiens
AILNVDEKNQV

SEQ ID NO: 89 - protein, 9 aa, Homo sapiens
RLFEEVLGV

SEQ ID NO: 90 - protein, 9 aa, Homo sapiens
YLDEVAFML

SEQ ID NO: 91 - protein, 11 aa, Homo sapiens
KLIDEDEPLFL

SEQ ID NO: 92 - protein, 9 aa, Homo sapiens
KLFEKSTGL

SEQ ID NO: 93 - protein, 11 aa, Homo sapiens
SLLEVNEASSV

SEQ ID NO: 94 - protein, 10 aa, Homo sapiens
GVYDGREHTV

SEQ ID NO: 95 - protein, 10 aa, Homo sapiens
GLYPVTLVGV

SEQ ID NO: 96 - protein, 9 aa, Homo sapiens
ALLSSVAEA

SEQ ID NO: 97 - protein, 9 aa, Homo sapiens
TLLEGISRA

SEQ ID NO: 98 - protein, 9 aa, Homo sapiens
SLIEESEEL

SEQ ID NO: 99 - protein, 9 aa, Homo sapiens
ALYVQAPTV

SEQ ID NO: 100 - protein, 10 aa, Homo sapiens
KLIYKDLVSV

SEQ ID NO: 101 - protein, 9 aa, Homo sapiens
ILQDGQFLV

SEQ ID NO: 102 - protein, 9 aa, Homo sapiens
SLLDYEVSI

SEQ ID NO: 103 - protein, 9 aa, Homo sapiens
LLGDSSFFL

SEQ ID NO: 104 - protein, 10 aa, Homo sapiens
VIFEGEPMYL

SEQ ID NO: 105 - protein, 9 aa, Homo sapiens
ALSYILPYL

SEQ ID NO: 106 - protein, 9 aa, Homo sapiens
FLFVDPELV

SEQ ID NO: 107 - protein, 11 aa, Homo sapiens
SEWGSPHAAVP

SEQ ID NO: 108 - protein, 9 aa, Homo sapiens
ALSELERVL

SEQ ID NO: 109 - protein, 9 aa, Homo sapiens
SLFESLEYL

SEQ ID NO: 110 - protein, 9 aa, Homo sapiens
KVLEYVIKV

SEQ ID NO: 111 - protein, 10 aa, Homo sapiens
VLLNEILEQV

SEQ ID NO: 112 - protein, 9 aa, Homo sapiens
SLLNQPKAV

SEQ ID NO: 113 - protein, 9 aa, Homo sapiens
KMSELQTYV

SEQ ID NO: 114 - protein, 11 aa, Homo sapiens
ALLEQTGDMSL

SEQ ID NO: 115 - protein, 11 aa, Homo sapiens
VIIKGLEEITV

SEQ ID NO: 116 - protein, 9 aa, Homo sapiens
KQFEGTVEI

SEQ ID NO: 117 - protein, 9 aa, Homo sapiens
KLQEEIPVL

SEQ ID NO: 118 - protein, 9 aa, Homo sapiens
GLAEFQENV

SEQ ID NO: 119 - protein, 9 aa, Homo sapiens
NVAEIVIHI

SEQ ID NO: 120 - protein, 9 aa, Homo sapiens
ALAGIVTNV

SEQ ID NO: 121 - protein, 12 aa, Homo sapiens
NLLIDDKGTIKL

SEQ ID NO: 122 - protein, 10 aa, Homo sapiens
VLMQDSRLYL

SEQ ID NO: 123 - protein, 9 aa, Homo sapiens
KVLEHVVRV

SEQ ID NO: 124 - protein, 9 aa, Homo sapiens
LLWGNLPEI

SEQ ID NO: 125 - protein, 9 aa, Homo sapiens
SLMEKNQSL

SEQ ID NO: 126 - protein, 9 aa, Homo sapiens
KLLAVIHEL

SEQ ID NO: 127 - protein, 10 aa, Homo sapiens
ALGDKFLLRV

SEQ ID NO: 128 - protein, 11 aa, Homo sapiens
FLMKNSDLYGA

SEQ ID NO: 129 - protein, 10 aa, Homo sapiens
KLIDHQGLYL

SEQ ID NO: 130 - protein, 12 aa, Homo sapiens
GPGIFPPPPPQP

SEQ ID NO: 131 - protein, 9 aa, Homo sapiens
ALNESLVEC

SEQ ID NO: 132 - protein, 9 aa, Homo sapiens
GLAALAVHL

SEQ ID NO: 133 - protein, 9 aa, Homo sapiens
LLLEAVWHL

SEQ ID NO: 134 - protein, 9 aa, Homo sapiens
SIIEYLPTL

SEQ ID NO: 135 - protein, 9 aa, Homo sapiens
TLHDQVHLL

SEQ ID NO: 136 - protein, 9 aa, Homo sapiens
SLLMWITQC

SEQ ID NO: 137 - protein, 11 aa, Homo sapiens
FLLDKPQDLSI

SEQ ID NO: 138 - protein, 10 aa, Homo sapiens
YLLDMPLWYL

SEQ ID NO: 139 - protein, 9 aa, Homo sapiens
GLLDCPIFL

SEQ ID NO: 140 - protein, 9 aa, Homo sapiens
VLIEYNFSI

SEQ ID NO: 141 - protein, 11 aa, Homo sapiens
TLYNPERTITV

SEQ ID NO: 142 - protein, 9 aa, Homo sapiens
AVPPPPSSV

SEQ ID NO: 143 - protein, 9 aa, Homo sapiens
KLQEELNKV

SEQ ID NO: 144 - protein, 11 aa, Homo sapiens
KLMDPGSLPPL

SEQ ID NO: 145 - protein, 9 aa, Homo sapiens
ALIVSLPYL

SEQ ID NO: 146 - protein, 9 aa, Homo sapiens
FLLDGSANV

SEQ ID NO: 147 - protein, 10 aa, Homo sapiens
ALDPSGNQLI

SEQ ID NO: 148 - protein, 9 aa, Homo sapiens
ILIKHLVKV

SEQ ID NO: 149 - protein, 9 aa, Homo sapiens
VLLDTILQL

SEQ ID NO: 150 - protein, 9 aa, Homo sapiens
HLIAEIHTA

SEQ ID NO: 151 - protein, 9 aa, Homo sapiens
SMNGGVFAV

SEQ ID NO: 152 - protein, 9 aa, Homo sapiens
MLAEKLLQA

SEQ ID NO: 153 - protein, 9 aa, Homo sapiens
YMLDIFHEV

SEQ ID NO: 154 - protein, 11 aa, Homo sapiens
ALWLPTDSATV

SEQ ID NO: 155 - protein, 9 aa, Homo sapiens
GLASRILDA

SEQ ID NO: 156 - protein, 9 aa, Homo sapiens
ALSVLRLAL

SEQ ID NO: 157 - protein, 9 aa, Homo sapiens
SYVKVLHHL

SEQ ID NO: 158 - protein, 9 aa, Homo sapiens
VYLPKIPSW

SEQ ID NO: 159 - protein, 9 aa, Homo sapiens
NYEDHFPLL

SEQ ID NO: 160 - protein, 9 aa, Homo sapiens
VYIAELEKI

SEQ ID NO: 161 - protein, 12 aa, Homo sapiens
VHFEDTGKTLLF

SEQ ID NO: 162 - protein, 9 aa, Homo sapiens
VLSPFILTL

SEQ ID NO: 163 - protein, 9 aa, Homo sapiens
HLLEGSVGV

SEQ ID NO: 164 - protein, 9 aa, Homo sapiens
ALREEEEGV

SEQ ID NO: 165 - protein, 10 aa, Homo sapiens
KEADPTGHSY

SEQ ID NO: 166 - protein, 9 aa, Homo sapiens
TLDEKVAEL

SEQ ID NO: 167 - protein, 10 aa, synthetic construct
  REGION 1..10: note = TAT peptide
GRKKKRRQRC

(19) United States
(12) Patent Application Publication
(10) Pub. No.: US 2025/0002855 A1
(43) Pub. Date: Jan. 2, 2025

(54) METHODS FOR EXPANDING T CELLS FOR THE TREATMENT OF CANCER AND RELATED MALIGNANCIES

(71) Applicant: Immatics US, Inc., Stafford, TX (US)

(72) Inventors: Melinda MATA, Missouri City, TX (US); Mamta KALRA, Stafford, TX (US); Ali MOHAMED, Stafford, TX (US); Steffen WALTER, Stafford, TX (US); Yannick BULLIARD, Thousand Oaks, CA (US)

(21) Appl. No.: 18/886,331

(22) Filed: Sep. 16, 2024

Related U.S. Application Data
(63) Continuation of application No. 17/281,095, filed on Mar. 29, 2021.
(60) Provisional application No. 63/082,881, filed on Sep. 24, 2020.
(60) Provisional application No. 63/038,008, filed on Jun. 11, 2020.
(60) Provisional application No. 62/980,844, filed on Feb. 24, 2020.

(51) Int. Cl. C12N 5/0783; A61K 39/00; C07K 14/725

(52) U.S. Cl. CPC C12N 5/0638; A61K 39/4611; A61K 39/4632; A61K 39/464489; C07K 14/7051; C12N 5/0636; C12N 2500/42; C12N 2501/2301; C12N 2501/2302; C12N 2501/2312; C12N 2501/2315; C12N 2501/2318; C12N 2501/2321; C12N 2501/24; C12N 2502/1157; C12N 2502/30; C12N 2502/99; C12N 2510/00

(57) ABSTRACT

An in vitro method of expanding γδ T cells includes isolating γδ T cells from a blood sample of a human subject, activating the isolated γδ T cells in the presence of an aminobisphosphonate and/or a feeder cell and at least one cytokine, expanding the activated γδ T cells, and optionally restimulating the expanded γδ T cells.

### CROSS-REFERENCE TO RELATED APPLICATIONS

This application is a continuation application of U.S. patent application Ser. No. 17/281,095, filed Mar. 29, 2021, which is the National Stage entry of International Application No. PCT/US2021/019252, filed Feb. 23, 2021, which claims priority to U.S. Provisional Application No. 62/980,844, filed Feb. 24, 2020, U.S. Provisional Application No. 63/038,008, filed Jun. 11, 2020, and U.S. Provisional Application No. 63/082,881, filed Sep. 24, 2020; the contents of each of which are incorporated by reference in their entirety.

### REFERENCE TO SEQUENCE LISTING SUBMITTED AS AN XML FILE (.xml)

A Sequence Listing is submitted herewith as an XML file named “______” created on ______ and having a size of ______ bytes as permitted under 37 C.F.R. § 1.821 (c). The material in the aforementioned file is hereby incorporated by reference in its entirety.

### BACKGROUND

### Field

The present disclosure relates to relates to expansion and activation of T cells. In an aspect, the present disclosure relates to expansion and activation of γδ T cells that may be used for transgene expression. In another aspect, the disclosure relates to expansion and activation of γδ T cells while depleting α- and/or β-TCR positive cells. T cell populations comprising expanded γδ T cell and depleted or reduced α- and/or β-TCR positive cells are also provided for by the instant disclosure. The disclosure further provides for methods of using the disclosed T cell populations.

γδ T cells represent a subset of T cells expressing the γδ TCR instead of the αβ TCR. γδ T cells can be divided into two primary subsets—the tissue-bound V [symbol] 2-negative cells and the peripheral circulating Vδ2positive cells, more specifically V [symbol] 9 [symbol] 2. Both subsets have been shown to have anti-viral and anti-tumor activities. Unlike the conventional αβ TCR expressing cells, γδ TCR-expressing cells recognize their targets independent of the classical MHC I and II. Similar to natural killer (NK) T cells, γδ T cells express NKG2D, which binds to the non-classical MHC molecules, i.e., MHC class I polypeptide-related sequence A (MICA) and MHC class I polypeptide-related sequence B (MICB), present on stressed cells and/or tumor cells. γδ TCR recognizes a variety of ligands, e.g., stress and/or tumor-related phosphoantigen. γδ T cells mediate direct cytolysis of their targets via multiple mechanisms, i.e., TRAIL, FasL, perforin and granzyme secretion. In addition, γδ T cells expressing CD16 potentiates antibody-dependent cell mediated cytotoxicity (ADCC).

A problem of γδ T cells, which may be generally present in an amount of only 1 to 5% in peripheral blood, is that the purity and number of the γδ T cells sufficient for medical treatment cannot be secured, especially if a small amount of blood is collected and then the cells therefrom are activated and/or proliferated. Increasing the amount of blood collection from a patient to secure the purity and number of the γδ T cells sufficient for medical treatment also poses a problem in that it imposes a great burden on the patient.

There remains a need for methods that could prepare sufficient number of γδ T cells as a commercially viable therapeutic product. A solution to this technical problem is provided by the embodiments characterized in the claims.

### BRIEF SUMMARY

The present application provides a method of expanding γδ T cells including isolating γδ T cells from a blood sample of a human subject, activating the isolated γδ T cells in the presence of a feeder cell and at least one cytokine, and expanding the activated γδ T cells.

The present disclosure further provides a method of expanding γδ T cells including isolating γδ T cells from a blood sample of a human subject, activating the isolated γδ T cells in the presence of at least one cytokine and one or more of 1) an aminobisphosphonate, 2) a feeder cell, or 3) an aminobisphosphonate and a feeder cell, expanding the activated γδ T cells, and restimulating the expanded γδ T cells.

In an aspect, the blood sample comprises leukapheresis product.

In an aspect, the blood sample comprises peripheral blood mononuclear cells (PBMC).

In some aspects, the activating is in the presence of an aminobisphosphonate.

In some aspects, the aminobisphosphonate comprises pamidronic acid, alendronic acid, zoledronic acid, risedronic acid, ibandronic acid, incadronic acid, a salt thereof and/or a hydrate thereof.

In some aspects, the aminobisphosphonate comprises zoledronic acid.

In some aspects, the at least one cytokine is selected from the group consisting of interleukin (IL)-1, IL-2, IL-12, IL-18, IL-15, IL-21, interferon (IFN)-α, and IFN-β.

In some aspects, the at least one cytokine comprises IL-2 and IL-15.

In an aspect, the isolating comprises contacting the blood sample with anti-α and anti-β T cell receptor (TCR) antibodies and depleting α- and/or β-TCR positive cells from the blood sample.

In an aspect, the feeder cell is a tumor cell or a lymphoblastoid cell line.

In some aspects, the tumor cell is a K562 cell.

In some aspects, the tumor cell is an engineered tumor cell comprising at least one recombinant protein.

In some aspects, the at least one recombinant protein is selected from the group consisting of CD86, 4-1BBL, IL-15, and any combination thereof.

In some aspects, the IL-15 is membrane bound IL-15.

In some aspects, the at least one recombinant protein is 4-1BBL and/or membrane bound IL-15.

In some aspects, the feeder cell is irradiated.

In some aspects, the isolated γδ T cells and the feeder cell are mixed in a ratio of from about 1:1 to about 50:1 (feeder cell: isolated γδ T cells). In some aspects, the isolated γδ T cells and the feeder cell is present in a ratio of from about 2:1 to about 20:1 (feeder cell: isolated γδ T cells). In some aspects, the isolated γδ T cells and the feeder cell is present in a ratio of about 1:1, about 1:5:1, about 2:1, about 3:1, about 4:1, about 5:1, about 6:1, about 7:1, about 8:1, about 9:1, about 10:1, about 11:1, about 12:1, about 13:1, about 14:1, about 15:1, about 20:1, about 25:1, about 30:1, about 35:1, about 40:1, about 45:1 or about 50:1 (feeder cells: isolated γδ T cells).

In an aspect, the method of the present application further comprises transducing the activated γδ T cells with a recombinant viral vector prior to the expanding.

In an aspect, the expanding is in the absence of an aminobisphosphonate and in the presence of at least one cytokine, such as, for example, IL-2 and/or IL-15.

In some aspects, the method of the present disclosure includes restimulating the expanded γδ T cells.

In some aspects, the restimulating comprises contacting the expanded γδ T cells with a further feeder cell which can be the same or different from the feeder cell used during activation (if present).

In some aspects, the expanded γδ T cells and the further feeder cell are mixed in a ratio of from about 1:1 to about 50:1 (further feeder cell: expanded γδ T cells). In some aspects, the expanded γδ T cells and the further feeder cell is present in a ratio of from about 2:1 to about 20:1 (further feeder cell: expanded γδ T cells). In some aspects, the expanded γδ T cells and the further feeder cell is present in a ratio of about 1:1, about 1:5:1, about 2:1, about 3:1, about 4:1, about 5:1, about 6:1, about 7:1, about 8:1, about 9:1, about 10:1, about 11:1, about 12:1, about 13:1, about 14:1, about 15:1, about 20:1, about 25:1, about 30:1, about 35:1, about 40:1, about 45:1 or about 50:1 (further feeder cells: expanded γδ T cells).

In an aspect, the further feeder cell is selected from the group consisting of monocytes, PBMCs, and combinations thereof.

In some aspects, the further feeder cell is autologous to the human subject.

In some aspects, the further feeder cell is allogenic to the human subject.

In some aspects, the further feeder cell is depleted of αβ T cells.

In some aspects, the further feeder cell is contacted or pulsed with an aminobisphosphonate,

such as zoledronic acid, prior to restimulation.

In an aspect, the further feeder cell is a tumor cell or a lymphoblastoid cell line.

In some aspects, the tumor cell is a K562 cell.

In some aspects, the tumor cell is an engineered tumor cell comprising at least one recombinant protein.

In some aspects, the at least one recombinant protein is selected from the group consisting of CD86, 4-1BBL, IL-15, and any combination thereof.

In some aspects, the IL-15 is membrane bound IL-15.

In some aspects, the further feeder cell is irradiated.

In an aspect, the present application relates to a population of expanded γδ T cells prepared by the methods of the present disclosure, in which the density of the expanded γδ T cells is at least about 1×10⁵ cells/ml, at least about 1×10⁶ cells/ml, at least about 1×10⁷ cells/ml, at least about 1×10⁸ cells/ml, or at least about 1×10⁹ cells/ml.

In an aspect, the present application relates to a method of treating cancer, comprising administering to a patient in need thereof an effective amount of the expanded γδ T cells prepared by the methods of the present disclosure.

In an aspect, the cancer is selected from the group consisting of acute lymphoblastic leukemia, acute myeloid leukemia, adrenocortical carcinoma, AIDS-related cancers, AIDS-related lymphoma, anal cancer, appendix cancer, astrocytomas, neuroblastoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, μleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, Merkel cell skin carcinoma, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom's macroglobulinemia, and Wilms tumor.

In an aspect, the cancer is melanoma.

In an aspect, the present application relates to a method of treating an infectious disease, comprising administering to a patient in need thereof an effective amount of the expanded γδ T cells prepared by the methods of the present disclosure.

In an aspect, the infectious disease is selected from the group consisting of dengue fever, Ebola, Marburg virus, tuberculosis (TB), meningitis, and syphilis.

In an aspect, the present application relates to a method of treating an autoimmune disease, comprising administering to a patient in need thereof an effective amount of the expanded γδ T cells prepared by the methods of the present disclosure.

In an aspect, the autoimmune disease is selected from the group consisting of Arthritis, Chronic obstructive pulmonary disease, Ankylosing Spondylitis, Crohn's Disease (one of two types of idiopathic inflammatory bowel disease “IBD”), Dermatomyositis, Diabetes mellitus type 1, Endometriosis, Goodpasture's syndrome, Graves' disease, Guillain-Barre syndrome (GBS), Hashimoto's disease, Hidradenitis suppurativa, Kawasaki disease, IgA nephropathy, Idiopathic thrombocytopenia purpura, Interstitial cystitis, Lupus erythematosus, Mixed Connective Tissue Disease, Morphea, Myasthenia gravis, Narcolepsy, Neuromyotonia, Pemphigus vulgaris, Pernicious anaemia, Psoriasis, Psoriatic Arthritis, Polymyositis, Primary biliary cirrhosis, Relapsing polychondritis, Rheumatoid arthritis, Schizophrenia, Scleroderma, Sjogren's syndrome, Stiff person syndrome, Temporal arteritis (also known as “giant cell arteritis”), Ulcerative Colitis (one of two types of idiopathic inflammatory bowel disease “IBD”), Vasculitis, Vitiligo, and Wegener's granulomatosis.

In an aspect, the present application relates to a method of preparing γδ T cells including isolating γδ T cells from a blood sample of a human subject, activating the isolated γδ T cells in the absence of a feeder cell, introducing a vector comprising a nucleic acid encoding a T cell receptor (TCR) or a chimeric antigen receptor (CAR) into the activated γδ T cells, and expanding the transduced γδ T cells in the presence of a feeder cell.

In another aspect, the activating, the transducing, and/or the expanding may be performed in the presence of at least one cytokine selected from the group consisting of interleukin (IL)-1, IL-2, IL-12, IL-15, IL-18, IL-21, interferon (IFN)-α, and IFN-β.

In another aspect, the feeder cell may be a human cell, a non-human cell, a virus-infected cell, a non-virus infected cell, a cell extract, a particle, a bead, a filament, or a combination thereof.

In another aspect, the feeder cell may include peripheral blood mononuclear cells (PBMCs) and/or lymphoblastoid cells (LCLs).

In another aspect, the activating, the transducing, and/or the expanding may be performed in the presence of OKT3.

In another aspect, the expanded γδ T cells may include δ1 and/or δ2 T cells.

In another aspect, the vector may be a viral vector or a non-viral vector.

In another aspect, the vector may include a nucleic acid encoding a TCR and a nucleic acid encoding CD8αβ or CD8α.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 shows allogenic T cell therapy according to an embodiment of the present disclosure. Allogenic T cell therapy may include collecting γδ T cells from healthy donors, engineering γδ T cells by viral transduction of exogenous genes of interest, such as exogenous TCRs, followed by cell expansion, harvesting the expanded engineered γδ T cells, which may be cryopreserved as T-cell products, before infusing into patients.

FIG. 2 shows γδ T cell manufacturing according to an embodiment of the present disclosure. γδ T cell manufacturing may include collecting or obtaining white blood cells or PBMC, e.g., leukapheresis product, depleting αβ T cells from PBMC or leukapheresis product, followed by activation, transduction, expansion, and optionally, re-stimulation of γδ T cells.

FIGS. 3A and 3B show the effect of re-stimulation with autologous monocytes on the expansion of γδ T cells. FIG. 3A shows the re-stimulation process. Briefly, on Day 0, the αβ-TCR expressing T cell (including CD4+ and CD8+ T cells)-depleted peripheral blood mononuclear cells (PBMC) (“γδ T cells”) were activated in the presence of zoledronate (ZOL) (5 μM), IL-2 (100 U/ml), and IL-15 (100 ng/ml). On Day 3, the activated γδ T cells were mock transduced. On Day 4, the mock-transduced cells are expanded. On Day 7, the expanded cells were re-stimulated with autologous monocytes obtained by CD14+selection from PBMC (Miltenyi) in the presence of ZOL (100 μM) for 4 hours at a ratio of 10 (monocytes):1 (γδ T cells).

FIG. 3B shows re-stimulation with monocytes increases fold-expansion of γδ T cells obtained from two donors (D1 and D2) as compared with that without re-stimulation. The fold expansion of the re-stimulated cells decreases after 10 days. By 14 days, the fold expansion of the re-stimulated cells decreases to fold expansion similar to that without re-stimulation.

FIGS. 4A and 4B show the effect of re-stimulation with irradiated autologous monocytes on the expansion of γδ T cells. FIG. 4A shows the re-stimulation process. Briefly, on Day 0, the αβ-TCR expressing T cells (including CD4+ and CD8+ T cells) depleted peripheral blood mononuclear cells (PBMC) (“γδ T cells”) were activated in the presence of zoledronate (ZOL) (5 μM), IL-2 (100 U/ml), and IL-15 (100 ng/ml). On Day 2, the activated γδ T cells were mock transduced. On Day 3, the mock-transduced cells are expanded. On Day 7, the expanded cells were re-stimulated with irradiated (100 Gy) autologous αβ-TCR expressing T cells depleted PBMC in the presence of ZOL (100 μM) for 4 hours at a ratio of 5:1 or 10:1 (αβ-TCR expressing T cells depleted PBMC: γδ T cells).

FIG. 4B shows re-stimulation with αβ-TCR expressing T cells depleted PBMC at 5:1 and 10:1 ratios increases fold-expansion of γδ T cells obtained from two donors (D1 and D2) as compared with that without re-stimulation.

FIG. 5 shows the expansion process used to generate the data presented in FIGS. 6-11. Briefly, on Day 0, the αβ-TCR expressing T cells (including CD4+ and CD8+ T cells) depleted peripheral blood mononuclear cells (PBMC) (“γδ T cells”) were activated in the presence of zoledronate (ZOL) (5 μM), IL-2 (100 U/ml), and IL-15 (100 ng/ml). On Day 2, the activated γδ T cells were mock transduced. On Day 3, the mock-transduced cells are expanded. On Day 7 and on Day 14, the expanded cells were re-stimulated with either 1) autologous monocytes (obtained by CD14+selection from PBMC (Miltenyi) and pulsed with ZOL (100 μM) for 4 hours) at a ratio of 1:1, 5:1 or 10:1 (monocytes: γδ T cells) or 2) irradiated (100 Gy) autologous αβ-TCR expressing T cells depleted PBMC (pulsed with ZOL (100 μM) for 4 hours) at a ratio of 10:1 or 20:1 (αβ depleted PBMC: γδ T cells).

FIGS. 6A and 6B show the effect of multiple re-stimulations with autologous monocytes or irradiated autologous αβ depleted PBMC on the expansion of γδ T cells from two donors (D1 (FIG. 6A) and D2 (FIG. 6B)). γδ T cells were activated and expanded as shown in FIG. 5.

FIGS. 7A-7C show the effect of multiple re-stimulations with autologous monocytes or irradiated autologous αβ depleted PBMC on the expansion of γδ T cells from one donor. γδ T cells were activated and expanded as shown in FIG. 5. FIG. 7A shows fold-expansion of total γδ T cells, FIG. 7B shows fold-expansion of δ2 T cells, and FIG. 7C shows fold-expansion of δ1 T cells.

FIGS. 8A-8C show the effect of multiple re-stimulations with autologous monocytes or irradiated autologous αβ depleted PBMC on the expansion of γδ T cells from a second donor. γδ T cells were activated and expanded as shown in FIG. 5. FIG. 8A shows fold-expansion of total γδ T cells, FIG. 8B shows fold-expansion of δ2 T cells, and FIG. 8C shows fold-expansion of δ1 T cells.

FIG. 9 shows that multiple re-stimulations with autologous monocytes or irradiated autologous αβ depleted PBMC does not significantly alter the memory phenotype of expanded γδ T cells. γδ T cells from one donor were activated and expanded as shown in FIG. 5, harvested on Day 21, and analyzed by flow cytometry to determine memory phenotype by detection of CD45, CD27, and CCR7 on the cell surface. A slight increase in CD27 expression was detected in expanded γδ T cells re-stimulated with 10:1 monocytes.

FIG. 10 shows that multiple re-stimulations with autologous monocytes or irradiated autologous αβ depleted PBMC does not significantly alter the memory phenotype of expanded γδ T cells. γδ T cells from a second donor were activated and expanded as shown in FIG. 5, harvested on Day 21, and analyzed by flow cytometry to determine memory phenotype by detection of CD45, CD27, and CCR7 on the cell surface. A slight increase in CD27 expression was detected in expanded γδ T cells re-stimulated with 10:1 monocytes.

FIG. 11A and 11B show the effect of multiple re-stimulations with autologous monocytes or irradiated autologous αβ depleted PBMC on viability of expanded γδ T cells. 8 T cells from two donors were activated and expanded as shown in FIG. 5, harvested on Day 21, and analyzed by flow cytometry to determine percentage of live cells within the total γδ T cell population. Results from donor 1 is shown in FIG. 11A and results from donor 2 is shown in FIG. 11B.

FIGS. 12A and 12B show the effect of co-culture of engineered tumor-derived cells on γδ T cells. Briefly, on Day 0, the αβ-TCR expressing T cells (including CD4+ and CD8+ T cells) depleted peripheral blood mononuclear cells (PBMC) (“γδ T cells”) were activated in the presence of zoledronate (ZOL) (5 μM), IL-2 (100 U/ml), and IL-15 (100 ng/ml). Irradiated tumor-derived cells (K562) were added in a 2:1 ratio (tumor-derived cells: γδ T cells) to some samples in either the presence or absence of ZOL. Other samples were cultured on anti-CD28 or anti-CD27 mAb-coated μlates. On Day 3, the activated γδ T cells were mock transduced. On Day 4, the mock-transduced cells were expanded. Expanded cells were frozen on Day 21.

FIG. 12A and 12B shows γδ T cells obtained from two donors (D1 (FIG. 12A) and D2 (FIG. 12B)) stimulated with irradiated tumor-derived cells +/−ZOL has higher fold expansion than that stimulated with anti-CD28 antibody+ZOL, anti-CD27 antibody+ZOL, and ZOL alone (control).

FIGS. 13A-C show results from co-culture of various tumor-derived cells during activation of γδ T cells. FIG. 13A shows fold expansion of γδ T cells obtained from two donors (D1 (top panel) and D2 (bottom panel)) activated on Day 0 in the presence of zoledronate (ZOL) (5 μM), IL-2 (100 U/ml), and IL-15 (100 ng/ml): 1) in the absence of tumor-derived cells (control); 2) with wild-type tumor-derived cells (K562 WT); 3) with modified tumor-derived cells (K562 variant 1); 4) with modified tumor-derived cells (K562 variant 2); 5) with modified tumor-derived cells (K562 variant 2) in the absence of ZOL; and 6) with modified tumor-derived cells (K562 variant 2) in the absence of ZOL with re-stimulation (K562 variant 2+IL-2+IL-15) on Days 7 and 14. FIGS. 13B and 13C show expansion of both δ1 (left panel) and δ2 (right panel) T cells in donor 1 (FIG. 13B) and donor 2 (FIG. 13C).

FIGS. 14A and 14B show results from co-culture of various tumor-derived cells during activation of γδ T cells. FIG. 14A and 14B show percentage of γδ T cells present within the entire live cell population. Briefly, cells obtained from two donors (D1 (FIG. 14A) and D2 (FIG. 14B)) were activated on Day 0 in the presence of zoledronate (ZOL) (5 μM), IL-2 (100 U/ml), and IL-15 (100 ng/ml): 1) in the absence of tumor-derived cells (control); 2) with wild-type tumor-derived cells (K562 WT); 3) with modified tumor-derived cells (K562 variant 1); 4) with modified tumor-derived cells (K562 variant 2); 5) with modified tumor-derived cells (K562 variant 2) in the absence of ZOL; and 6) with modified tumor-derived cells (K562 variant 2) in the absence of ZOL with re-stimulation (K562 variant 2+IL-2+IL-15) on Days 7 and 14.

FIG. 15 shows that lack of zoledronate in the culture results in a polyclonal population (both 81and 82γδ T cells) compared to conditions in which zoledronate was in the culture. Briefly, cells obtained from two donors (D1 (top panels) and D2 (bottom panels)) were activated on Day 0 in the presence of zoledronate (ZOL) (5 μM), IL-2 (100 U/ml), and IL-15 (100 ng/ml): 1) in the absence of tumor-derived cells (control); 2) with wild-type tumor-derived cells (K562); 3) with modified tumor-derived cells (K562 variant 2) in the absence of ZOL; 4) with modified tumor-derived cells (K562 variant 2) in the absence of ZOL with re-stimulation (K562 variant 2+IL-2+IL-15) on Days 7 and 14, 5) with modified tumor-derived cells (K562 variant 2), and 6) with modified tumor-derived cells (K562 variant 1). Cells were harvested on Day 21 and analyzed by flow cytometry to determine δ1 and δ2 populations.

FIG. 16 shows that tumor-derived co-culture does not alter the memory phenotype of expanded γδ T cells. Briefly, cells obtained from two donors (D1 (top panels) and D2 (bottom panels)) were activated on Day 0 in the presence of zoledronate (ZOL) (5 μM), IL-2 (100 U/ml), and IL-15 (100 ng/ml): 1) in the absence of tumor-derived cells (control); 2) with wild-type tumor-derived cells; 3) with tumor-derived cells engineered to express 4-1BBL and membrane-bound IL-15 (mbIL15) in the absence of ZOL; 4) with tumor-derived cells expressing 4-1BBL and mbIL15 in the absence of ZOL with re-stimulation (tumor-derived cells expressing 4-1BBL and mbIL15+IL-2+IL-15) on Days 7 and 14, 5) with tumor-derived cells expressing 4-1BBL and mbIL15, and 6) with tumor-derived cells expressing CD86. Cells were harvested on Day 21 and analyzed by flow cytometry to determine memory phenotype by detection of CD45, CD27, and CCR7 on the cell surface.

FIGS. 17A and 17B show the effect of multiple re-stimulations with irradiated allogenic PBMC+/−LCL on the expansion of γδ T cells from two donors (D1 (FIG. 17A) and D2 (FIG. 17B)). Briefly, cells obtained from two donors (D1 and D2) were activated on Day 0 in the presence of zoledronate (ZOL) (5 μM), IL-2 (100 U/ml), and IL-15 (100 ng/ml), mock transduced on Day 2 and expanded on Day 3. On Day 7 and on Day 14, the expanded cells were re-stimulated with: 1) control (100 U/ml IL-2+100 ng/ml IL-15); 2) PBMC+LCL+OKT3 (25×10⁶ irradiated allogenic PBMCs pooled from 2-3 donors+5×10⁶ irradiated LCL+30ng/ml sOTK3+50 U/ml IL-2); 3) PBMC (25×10⁶ irradiated allogenic PBMCs pooled from 2-3 donors+50U/ml IL-2); 4) LCL (5×10⁶ irradiated LCL+50U/ml IL-2); or 5) OKT3 (30ng/ml sOTK3+50U/ml IL-2).

FIGS. 18A-C show the effect of multiple re-stimulations with irradiated allogenic PBMC+/−LCL on the expansion of γδ T cells from two donors. γδ T cells were activated and expanded as described above for FIGS. 17A-B. FIGS. 18A and 18B show fold-expansion of δ1 T cells from the two donors. FIG. 18C shows the flow cytometry results on Day 21 from the two donors from the control treatment (IL-2+IL-15) and the PBMC+LCL+OKT3 re-stimulation treatment.

FIGS. 19A and 19B show the memory phenotype of expanded γδ T cells from two donors re-stimulated with PBMC+/−LCL. Briefly, cells obtained from two donors (D1 and D2) were activated on Day 0in the presence of zoledronate (ZOL) (5 μM), IL-2 (100 U/ml), and IL-15 (100 ng/ml), mock transduced on Day 2 and expanded on Day 3. On Day 7, the expanded cells were re-stimulated with: 1) control (100 U/ml IL-2+100 ng/ml IL-15); 2) PBMC+LCL+OKT3 (25×10⁶ irradiated allogenic PBMCs pooled from 2-3 donors+5×10⁶ irradiated LCL+30 ng/ml OKT3+50 U/ml IL-2); 3) PBMC (25×10⁶ irradiated allogenic PBMCs pooled from 2-3 donors+50 U/ml IL-2); or 4) LCL (5×10⁶ irradiated LCL+50U/ml IL-2). Cells were harvested on Day 14 and analyzed by flow cytometry to determine memory phenotype by detection of CD45, CD27, and CCR7 on the cell surface.

FIGS. 20A and 20B show, against peptide-positive U2OS cells (FIG. 20A) or peptide-negative MCF7 cells (FIG. 20B), the killing activity of γδ T cells transduced with TCR (TCR-T) or without transduction (NT) prepared by various processes.

FIG. 21 shows T cell manufacturing process in accordance with one embodiment of the present disclosure.

FIGS. 22A-22D show fold expansion of γδ T cells prepared by control process (FIG. 22A), Process 1 (FIG. 22B), Process 2 (FIG. 22C), and Process 3 (FIG. 22D).

FIGS. 23A-23C show phenotype CD27+CD45RA− (FIG. 23A), CD62L+ (FIG. 23B), and CD57+ (FIG. 23C) of γδ T cells prepared by various processes.

FIGS. 24A-24D show % γδ T cells expressing PD1 (FIG. 24A), LAG3 (FIG. 24B), TIM3 (FIG. 24C), and TIGIT (FIG. 24D) prepared by various processes.

FIGS. 25A and 25B shows % γδ T cells expressing transgenes, e.g., TCR, (FIG. 25A) and copy number of integrated TCR (FIG. 25B) of γδ T cells prepared by various processes.

FIGS. 26A-26C show % γδ T cells expressing transgenes, e.g., CD8 and TCR that binds PRAME peptide/MHC complex, prepared by control process (FIG. 26A), Process 2 (FIG. 26B), and Process 3 (FIG. 26C).

FIG. 27A shows T cell manufacturing process in accordance with another embodiment of the present disclosure.

FIG. 27B shows fold expansion of γδ T cells prepared by various processes.

FIGS. 28A-28C show % γδ T cells expressing transgenes, e.g., CD8 and TCR, prepared by stimulation with K562 cells on Day 0 followed by transduction on Day 2 with viral vector encoding transgenes at 60 μl (FIG. 28A), 120 μl (FIG. 28B), and 240 μl (FIG. 28C).

FIG. 28D shows copy number of integrated transgenes in γδ T cells prepared by the processes shown in FIGS. 28A-28C.

FIG. 28E shows % γδ T cells expressing transgenes, e.g., CD8 and TCR, prepared by transduction with viral vector encoding transgenes on Day 2 at 60 μl followed by stimulation with K562 cells on Day 4.

FIG. 28F shows copy number of integrated transgene in γδ T cells prepared by the process shown in FIG. 28E.

FIG. 29 shows % γδ T cells expressing transgenes, e.g., CD8 and TCR, prepared by various processes.

FIG. 30 shows γδ T cell manufacturing process in accordance with another embodiment of the present disclosure.

FIGS. 31A-31D show killing activities of γδ T cells prepared by various processes against UACC257 cells (FIG. 31A), U2OS cells (FIG. 31B), A375 cells (FIG. 31C), and MCF7 cells (FIG. 31D).

FIGS. 32A-32C show IFNγ secretion from γδ T cells prepared by various processes against UACC257 cells (FIG. 32A), U2OS cells (FIG. 32B), and MCF7 cells (FIG. 32C).

FIGS. 33A-33C show TNFα secretion from γδ T cells prepared by various processes against UACC257 cells (FIG. 33A), U2OS cells (FIG. 33B), and MCF7 cells (FIG. 33C).

FIGS. 34A-34C show GM-CSF secretion from γδ T cells prepared by various processes against UACC257 cells (FIG. 34A), U2OS cells (FIG. 34B), and MCF7 cells (FIG. 34C).

FIGS. 35A and 35B show growth inhibition of UACC257 cells induced by γδ T obtained from 2 donors (Donor 1 (FIG. 35A) and Donor 2 (FIG. 35B)) prepared by various processes.

FIG. 36 shows % transgenes (CD8 and TCR)-expressing γδ T cells expressing PD1, LAG3, TIM3, or TIGIT prepared by various processes.

FIG. 37 shows γδ T cell manufacturing processes in accordance with some embodiments of the present disclosure.

FIG. 38 shows CD28+CD62L+ γδ T cells prepared by various processes.

FIGS. 39A-39C show fold expansion of γδ T cells obtained from 3 donors (SD01004687 (FIG. 39A), D155410 (FIG. 39B), and SD010000256 (FIG. 39C)) prepared by various processes.

FIGS. 40A-40C show % δ1 and δ2 T cells prepared by control process (FIG. 40A), HDACi+IL-21 (w1) (FIG. 40B), and HDACi+IL-21 (w2) (FIG. 40C).

FIG. 41A shows % CD28+CD62L+ γδ T cells prepared by various processes.

FIG. 41B shows % CD27+CD45RA-γδ T cells prepared by various processes.

FIG. 41C shows % CD57+γδ T cells prepared by various processes.

FIG. 42 shows γδ T cell manufacturing processes in accordance with some embodiments of the present disclosure.

FIGS. 43A and 43B show % γδ T cells obtained from 2 donors (D155410 (FIG. 43A) and SD010004867 (FIG. 43B)) expressing IL-2Rα, IL-2Rβ, IL-2Rγ, IL-7Rα, and IL-21R.

FIGS. 44A-44C show fold expansion of γδ T cells obtained from 3 donors (SD010004867 (FIG. 44A), D155410 (FIG. 44B), and SD010000256 (FIG. 44C)) prepared by various processes.

FIGS. 45A-45C show % δ1 and δ2 T cells prepared by IL-12+IL-18 prime (FIG. 45A), IL-2+IL-15 (FIG. 45B), and control process (FIG. 45C).

FIG. 46A shows % CD27+CD45RA-γδ T cells prepared by various processes.

FIG. 46B shows % CD28+CD62L+ γδ T cells prepared by various processes.

FIG. 46C shows % CD57+ γδ T cells prepared by various processes.

FIGS. 47A and 47B show % δ1 and δ2 T cells obtained from 2 donors (D148960 (FIG. 47A) and

SD010000723 (FIG. 47B)) prepared by various processes.

FIGS. 48A and 48B show % δ1 (FIG. 48A) and δ2 (FIG. 48B) T cells obtained from donor SD010000723 prepared by various processes.

FIGS. 49A and 49B show % δ1 (FIG. 49A) and δ2 (FIG. 49B) T cells obtained from donor D148960 prepared by various processes.

### DETAILED DESCRIPTION

Allogeneic T cell therapy may be based on genetically engineering allogeneic γδ T cells to express exogenous TCRs. In addition to the specific tumor recognition via the ectopic TCR or CAR, γδ T cells may have activity against numerous tumor types as described herein.

The term “γδ T-cells (gamma delta T-cells)” as used herein refers to a subset of T-cells that express a distinct T-cell receptor (TCR), γδ TCR, on their surface, composed of one y-chain and one 8-chain. The term “γδ T-cells” specifically includes all subsets of γδ T-cells, including, without limitation, Vδ1 and Vδ2, Vδ3 γδ T cells, as well as naive, effector memory, central memory, and terminally differentiated γδ T-cells. As a further example, the term “γδ T-cells” includes Vδ4, Vδ5, Vδ7, and Vδ8γδ T cells, as well as Vδ2, Vδ3, Vδ5, Vδ8, Vδ9, Vδ10, and Vδ11γδ T cells.

An “enriched” cell population or preparation refers to a cell population derived from a starting mixed cell population that contains a greater percentage of a specific cell type than the percentage of that cell type in the starting population. For example, a starting mixed cell population can be enriched for a specific γδ T-cell population. In one embodiment, the enriched γδ T-cell population contains a greater percentage of δ1 cells than the percentage of that cell type in the starting population. As another example, an enriched γδ T-cell population can contain a greater percentage of both δ1 cells and a greater percentage of δ3 cells than the percentage of that cell type in the starting population. As yet another example, an enriched γδ T-cell population can contain a greater percentage of both δ1 cells and a greater percentage of δ4 cells than the percentage of that cell type in the starting population. As yet another example, an enriched γδ T-cell population can contain a greater percentage of δ1 T cells, δ3 T cells, δ4 T cells, and δ5 T cells than the percentage of that cell type in the starting population. In another embodiment, the enriched γδ T-cell population contains a greater percentage of δ2 cells than the percentage of that cell type in the starting population. In yet another embodiment, the enriched γδ T-cell population contains a greater percentage of both δ1 cells and δ2 cells than the percentage of that cell type in the starting population. In all embodiments, the enriched γδ T-cell population contains a lesser percentage of αβ T-cell populations.

By “expanded” as used herein is meant that the number of the desired or target cell type (e.g., δ1 and/or δ2 T-cells) in the enriched preparation may be higher than the number in the initial or starting cell population. By “selectively expand” is meant that the target cell type (e.g., δ1 or δ2 T-cells) may be preferentially expanded over other non-target cell types, e.g., αβ T-cells or NK cells. In certain embodiments, the activating agents of the present application may selectively expand, e.g., engineered or non-engineered, δ1 T-cells without significant expansion of δ2 T-cells. In other embodiments, the activating agents of the present application may selectively expand, e.g., engineered or non-engineered, δ2 T-cells without significant expansion of δ1 T-cells. In certain embodiments, the activating agents of the present application may selectively expand, e.g., engineered or non-engineered, δ1 and δ3 T-cells without significant expansion of δ2 T-cells. In certain embodiments, the activating agents of the present application may selectively expand, e.g., engineered or non-engineered, δ1 and δ4 T-cells without significant expansion of δ2 T-cells. In certain embodiments, the activating agents of the present application may selectively expand, e.g., engineered or non-engineered, δ1, δ3, δ4 and δ5 T-cells without significant expansion of δ2 T-cells. In this context, the term “without significant expansion of” means that the preferentially expanded cell population are expanded at least 10-fold, preferably 100-fold, and more preferably 1,000-fold more than the reference cell population. Expanded T-cell populations may be characterized, for example, by magnetic-activated cell sorting (MACS) and/or fluorescence-activated cell sorting (FACS) staining for cell surface markers that distinguish between the different populations.

### Isolation of γδ T-Cells

In some aspects, the instant application may provide ex viγδ methods for expansion of engineered or non-engineered γδ T-cells. In some cases, the method may employ one or more (e.g., first and/or second) expansion steps that may not include a cytokine that favors expansion of a specific population of γδ T-cells, such as IL-4, IL-2, or IL-15, or a combination thereof. In some embodiments, the instant application may provide ex viγδ methods for producing enriched γδ T-cell populations from isolated mixed cell populations, including contacting the mixed cell population with one or more agents, which selectively expand δ1 T-cells; δ1 T-cells and δ3 T-cells; δ1 T-cells and δ4 T-cells; or δ1, 83, 84, and δ5 T cells by binding to an epitope specific of a δ1 TCR; a δ1 and δ4 TCR; or a δ1, δ3, δ4, and δ5 TCR respectively to provide an enriched γδ T cell population. In other aspects, the instant application may provide ex vivo methods for producing enriched γδ T-cell populations from isolated mixed cell populations, including contacting the mixed cell population with one or more agents, which selectively expand δ2 T-cells by binding to an epitope specific of a δ2 TCR to provide an enriched γδ T cell population.

In an aspect, the present disclosure relates to expansion and/or activation of T cells. In another aspect, the present disclosure relates to expansion and/or activation of γδ T cells in the absence of agents that bind to epitopes specific to γδ TCRs, such as antibodies against γδ TCRs. In another aspect, the present disclosure relates to expansion and/or activation of γδ T cells that may be used for transgene expression.

The disclosure further relates to expansion and activation of γδ T cells while depleting α- and/or β-TCR positive cells. T cell populations comprising expanded γδ T cell and depleted or reduced α- and/or β-TCR positive cells are also provided for by the instant disclosure. The disclosure further provides for methods of using the disclosed T cell populations.

In an aspect, methods for producing large-scale Good Manufacturing Practice (GMP)-grade TCR engineered Vδ982 T cells are provided herein.

In the absence of feeder cells, addition of IL-18 to purified [symbol] [symbol] [symbol] T cells enhances the expansion of BE T cells with notable increase in the amount of surface high affinity receptor for IL-2 (CD25 or IL-2Ra). Further, Amphotericin B, a Toll-like receptor 2 (TLR2) ligand, can activate [symbol] [symbol] T cells, CD8+ T cells, and NK cells and enhance the detection of surface expression of CD25, the high affinity IL-2Rα. Collectively, these observations highlight a critical role of IL-2 signaling in Zoledronate-mediated activation and expansion of V [symbol] 9 [symbol] 2 T cells. Thus, to maximize the availability of IL-2 for [symbol] [symbol] T cell proliferation via IL-2 signaling (or to minimize the sequestration of IL-2 by high number of [symbol] [symbol] T cells), methods of the present disclosure may include depleting [symbol] [symbol] T cells from normal PBMC using anti- [symbol] [symbol] TCR commercially available GMP reagents. As recombinant IL-18 is currently not available as a commercial GMP-reagent, methods of the present disclosure may supplement the culture with low dose Amphotericin B to increase CD25 surface expression to enhance IL-2 binding and signaling, which in turn may enhance IL-2 responsiveness during activation/expansion. In addition, IL-15 may be added because IL-15 has been shown to increase proliferation and survival of V [symbol] 9 [symbol] 2 T cells treated with IPP.

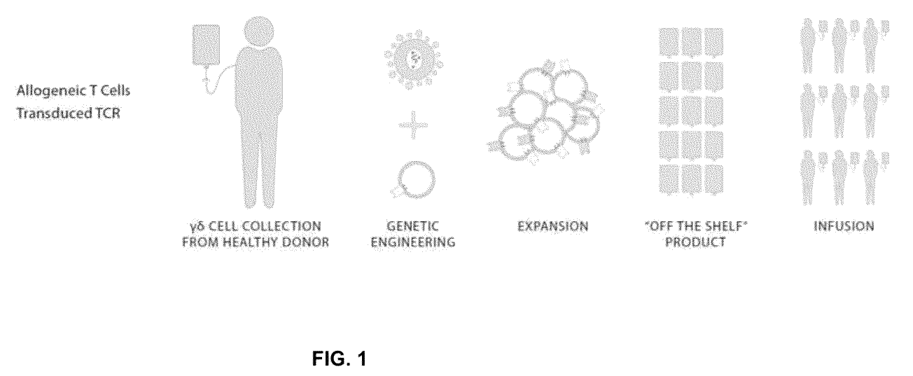

FIG. 1 shows an approach for adoptive allogenic T cell therapy that can deliver “off-the-shelf” T-cell products, such as γδ T cell products, for rapid treatment of eligible patients with a specific cancer expressing the target of interest in their tumors. This approach may include collecting γδ T cells from healthy donors, engineering γδ T cells by viral transduction of exogenous genes of interest, such as exogenous TCRs, followed by cell expansion, harvesting the expanded engineered γδ T cells, which may be cryopreserved as “off-the-shelf” T-cell products, before infusing into patients. This approach therefore may eliminate the need for personalized T cell manufacturing.

To isolate γδ T cells, in an aspect, γδ T cells may be isolated from a subject or from a complex sample of a subject. In an aspect, a complex sample may be a peripheral blood sample, a cord blood sample, a tumor, a stem cell precursor, a tumor biopsy, a tissue, a lymph, or from epithelial sites of a subject directly contacting the external milieu or derived from stem precursor cells. γδ T cells may be directly isolated from a complex sample of a subject, for example, by sorting γδ T cells that express one or more cell surface markers with flow cytometry techniques. Wild-type γδ T cells may exhibit numerous antigen recognition, antigen-presentation, co-stimulation, and adhesion molecules that can be associated with a γδ T cells. One or more cell surface markers, such as specific γδ TCRs, antigen recognition, antigen-presentation, ligands, adhesion molecules, or co-stimulatory molecules may be used to isolate wild-type γδ T cells from a complex sample. Various molecules associated with or expressed by γδ T-cells may be used to isolate γδ T cells from a complex sample. In another aspect, the present disclosure provides methods for isolation of mixed population of Vδ1+, Vδ2+, Vδ3+ cells or any combination thereof.

For example, peripheral blood mononuclear cells can be collected from a subject, for example, with an apheresis machine, including the Ficoll-Paque™ PLUS (GE Healthcare) system, or another suitable device/system. γδ T-cell(s), or a desired subpopulation of γδ T-cell(s), can be purified from the collected sample with, for example, with flow cytometry techniques. Cord blood cells can also be obtained from cord blood during the birth of a subject.

Positive and/or negative selection of cell surface markers expressed on the collected γδ T cells can be used to directly isolate γδ T cells, or a population of γδ T cells expressing similar cell surface markers from a peripheral blood sample, a cord blood sample, a tumor, a tumor biopsy, a tissue, a lymph, or from an epithelial sample of a subject. For instance, γδ T cells can be isolated from a complex sample based on positive or negative expression of CD2, CD3, CD4, CD8, CD24, CD25, CD44, Kit, TCR α, TCR β, TCR α, TCR δ, NKG2D, CD70, CD27, CD30, CD16, CD337 (NKp30), CD336 (NKp46), OX40, CD46, CCR7, and other suitable cell surface markers.

In an aspect, γδ T cells may be isolated from a complex sample that is cultured in vitro. In another aspect, whole PBMC population, without prior depletion of specific cell populations, such as monocytes, αβ T-cells, B-cells, and NK cells, can be activated and expanded. In another aspect, enriched γδ T cell populations can be generated prior to their specific activation and expansion. In another aspects, activation and expansion of γδ T cells may be performed without the presence of native or engineered APCs. In another aspects, isolation and expansion of γδ T cells from tumor specimens can be performed using immobilized γδ T cell mitogens, including antibodies specific to γδ TCR, and other γδ TCR activating agents, including lectins. In another aspect, isolation and expansion of γδ T cells from tumor specimens can be performed in the absence of γδ T cell mitogens, including antibodies specific to γδ TCR, and other γδ TCR activating agents, including lectins.

In an aspect, γδ T cells are isolated from leukapheresis of a subject, for example, a human subject. In another aspect, γδ T cells are not isolated from peripheral blood mononuclear cells (PBMC).

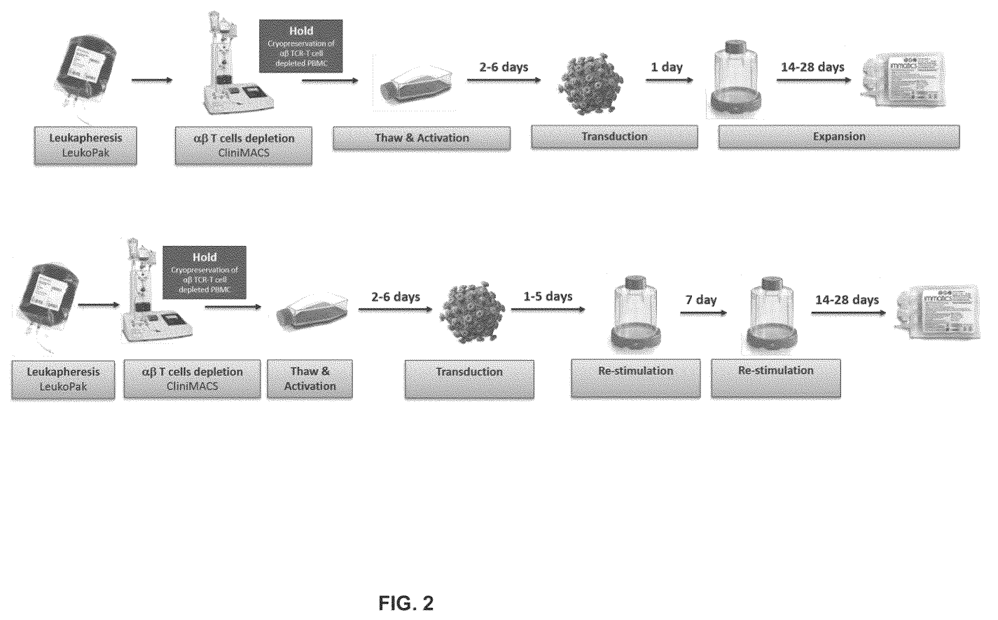

FIG. 2 shows γδ T cell manufacturing according to an embodiment of the present disclosure. This process may include collecting or obtaining white blood cells or PBMC from leukapheresis products. Leukapheresis may include collecting whole blood from a donor and separating the components using an apheresis machine. An apheresis machine separates out desired blood components and returns the rest to the donor's circulation. For instance, white blood cells, μlasma, and μlatelets can be collected using apheresis equipment, and the red blood cells and neutrophils are returned to the donor's circulation. Commercially available leukapheresis products may be used in this process. Another way to obtain white blood cells is to obtain them from the buffy coat. To isolate the buffy coat, whole anticoagulated blood is obtained from a donor and centrifuged. After centrifugation, the blood is separated into the μlasma, red blood cells, and buffy coat. The buffy coat is the layer located between the μlasma and red blood cell layers. Leukapheresis collections may result in higher purity and considerably increased mononuclear cell content than that achieved by buffy coat collection. The mononuclear cell content possible with leukapheresis may be typically 20 times higher than that obtained from the buffy coat. In order to enrich for mononuclear cells, the use of a Ficoll gradient may be needed for further separation.

To deplete αβ T cells from PBMC, αβ TCR-expressing cells may be separated from the PBMC by magnetic separation, e.g., using CliniMACS® magnetic beads coated with anti-αβ TCR antibodies, followed by cryopreserving αβ TCR-T cells depleted PBMC. To manufacture “off-the-shelf” T-cell products, cryopreserved αβ TCR-T cells depleted PBMC may be thawed and activated in small/mid-scale, e.g., 24 to 4-6 well μlates or T75/T175 flasks, or in large scale, e.g., 50 ml-100 liter bags, in the presence of aminobisphosphonate and/or isopentenyl pyrophosphate (IPP) and/or cytokines, e.g., interleukin 2 (IL-2), interleukin 15 (IL-15), and/or interleukin 18 (IL-18), and/or other activators, e.g., Toll-like receptor 2 (TLR2) ligand, for 1-10 days, e.g., 2-6 days.

In an aspect, the isolated γδ T cells can rapidly expand in response to contact with one or more antigens. Some γδ T cells, such as Vδ9Vδ2+ T cells, can rapidly expand in vitro in response to contact with some antigens, like prenyl-pyrophosphates, alkyl amines, and metabolites or microbial extracts during tissue culture. Stimulated γδ T-cells can exhibit numerous antigen-presentation, co-stimulation, and adhesion molecules that can facilitate the isolation of γδ T-cells from a complex sample. γδ T cells within a complex sample can be stimulated in vitro with at least one antigen for 1 day, 2 days, 3 days, 4 days, 5 days, 6 days, 7days, or another suitable period of time. Stimulation of γδ T cells with a suitable antigen can expand γδ T cell population in vitro.

Non-limiting examples of antigens that may be used to stimulate the expansion of γδ T cells from a complex sample in vitro may include, prenyl-pyrophosphates, such as isopentenyl pyrophosphate (IPP), alkyl-amines, metabolites of human microbial pathogens, metabolites of commensal bacteria, methyl-3-butenyl-1-pyrophosphate (2M3B1PP), (E)-4-hydroxy-3-methyl-but-2-enyl pyrophosphate (HMB-PP), ethyl pyrophosphate (EPP), farnesyl pyrophosphate (FPP), dimethylallyl phosphate (DMAP), dimethylallyl pyrophosphate (DMAPP), ethyl-adenosine triphosphate (EPPPA), geranyl pyrophosphate (GPP), geranylgeranyl pyrophosphate (GGPP), isopentenyl-adenosine triphosphate (IPPPA), monoethyl phosphate (MEP), monoethyl pyrophosphate (MEPP), 3-formyl-1-butyl-pyrophosphate (TUBAg 1), X-pyrophosphate (TUBAg 2), 3-formyl-1-butyl-uridine triphosphate (TUBAg 3), 3-formyl-1-butyl-deoxythymidine triphosphate (TUBAg 4), monoethyl alkylamines, allyl pyrophosphate, crotoyl pyrophosphate, dimethylallyl-γ-uridine triphosphate, crotoyl-γ-uridine triphosphate, allyl-γ-uridine triphosphate, ethylamine, isobutylamine, sec-butylamine, iso-amylamine and nitrogen containing bisphosphonates.

Activation and expansion of γδ T cells can be performed using activation and co-stimulatory agents described herein to trigger specific γδ T cell proliferation and persistence populations. In an aspect, activation and expansion of γδ T-cells from different cultures can achieve distinct clonal or mixed polyclonal population subsets. In another aspect, different agonist agents can be used to identify agents that provide specific γδ activating signals. In another aspect, agents that provide specific γδ activating signals can be different monoclonal antibodies (MAbs) directed against the γδ TCRs. In another aspect, companion co-stimulatory agents to assist in triggering specific γδ T cell proliferation without induction of cell energy and apoptosis can be used. These co-stimulatory agents can include ligands binding to receptors expressed on γδ cells, such as NKG2D, CD161, CD70, JAML, DNAX accessory molecule-1 (DNAM-1), ICOS, CD27, CD137, CD30, HVEM, SLAM, CD122, DAP, and CD28. In another aspect, co-stimulatory agents can be antibodies specific to unique epitopes on CD2 and CD3 molecules. CD2 and CD3 can have different conformation structures when expressed on αβ or γδ T-cells. In another aspect, specific antibodies to CD3 and CD2 can lead to distinct activation of γδ T cells.

In some aspects, activation and/or expansion of γδ T cells can be performed in the presence of a feeder cell, such as a tumor cell, for example, a K562 cell or a lymphoblastoid cell (LCL). In some aspects, the feeder cell is modified to express one or more co-stimulatory agents, such as, for example, CD86, 4-1BBL, IL-15, and membrane-bound IL-15 (mbIL-15). In some aspects, the feeder cell may be an autologous cell, such as a monocyte or PBMC. The feeder cell may be an irradiated feeder cell, such as a γ-irradiated feeder cell. In some aspects, the feeder cells are co-cultured with the γδ T cells during activation. In some aspects, the feeder cells are co-cultured with the γδ T cells during expansion, for example, in one or more re-stimulation steps. The feeder cells used during activation can be the same or different from the feeder cells used during expansion.

In some aspects, the γδ T cells and the feeder cell is present in a ratio of from about 1:1 to about 50:1 (feeder cells: γδ T cells). In some aspects, the γδ T cells and the feeder cell is present in a ratio of from about 2:1 to about 20:1 (feeder cells: γδ T cells). In some aspects, the γδ T cells and the feeder cell is present in a ratio of about 1:1, about 1:5:1, about 2:1, about 3:1, about 4:1, about 5:1, about 6:1, about 7:1, about 8:1, about 9:1, about 10:1, about 11:1, about 12:1, about 13:1, about 14:1, about 15:1, about 20:1, about 25:1, about 30:1, about 35:1, about 40:1, about 45:1 or about 50:1 (feeder cells: γδ T cells).

A population of γδ T-cells may be expanded ex viγδ prior to engineering of the γδ T-cell. Non-limiting example of reagents that can be used to facilitate the expansion of a γδ T-cell population in vitro may include anti-CD3 or anti-CD2, anti-CD27, anti-CD30, anti-CD70, anti-OX40 antibodies, IL-2, IL-15, IL-12, IL-9, IL-33, IL-18, or IL-21, CD70 (CD27 ligand), phytohaemagglutinin (PHA), concavalin A (ConA), pokeweed (PWM), protein peanut agglutinin (PNA), soybean agglutinin (SBA), lens culinaris agglutinin (LCA), pisum sativum agglutinin (PSA), helix pomatia agglutinin (HPA), vicia graminea Lectin (VGA), or another suitable mitogen capable of stimulating T-cell proliferation.

The ability of γδ T cells to recognize a broad spectrum of antigens can be enhanced by genetic engineering of the γδ T cells. In an aspect, γδ T cell can be engineered to provide a universal allogeneic therapy that recognizes an antigen of choice in vivo. Genetic engineering of the γδ T-cells may include stably integrating a construct expressing a tumor recognition moiety, such as αβ TCR, γδ TCR, chimeric antigen receptor (CAR), which combines both antigen-binding and T-cell activating functions into a single receptor, an antigen binding fragment thereof, or a lymphocyte activation domain into the genome of the isolated γδ T-cell(s), a cytokine (IL-15, IL-12, IL-2. IL-7. IL-21, IL-18, IL-19, IL-33, IL-4, IL-9, IL-23, IL1B) to enhance T-cell proliferation, survival, and function ex viγδ and in vivo. Genetic engineering of the isolated γδ T-cell may also include deleting or disrupting gene expression from one or more endogenous genes in the genome of the isolated γδ T-cells, such as the MHC locus (loci).

T cell manufacturing methods disclosed herein may be useful for expanding T cells modified to express high affinity T cell receptors (engineered TCRs) or chimeric antigen receptors (CARs) in a reliable and reproducible manner. In one embodiment, T cell may be genetically modified to express one or more engineered TCRs or CARs. As used herein, T cells may be αβ T cells, γδ T cells, or natural killer T cells.

### Engineered TCRs

Naturally occurring T cell receptors comprise two subunits, an α-subunit and a β-subunit, each of which is a unique protein produced by recombination event in each T cell's genome. Libraries of TCRs may be screened for their selectivity to particular target antigens. In this manner, natural TCRs, which have a high-avidity and reactivity toward target antigens may be selected, cloned, and subsequently introduced into a population of T cells used for adoptive immunotherapy.

In one embodiment, T cells may be modified by introducing a polynucleotide encoding a subunit of a TCR that has the ability to form TCRs that confer specificity to T cells for tumor cells expressing a target antigen. In particular embodiments, the subunits may have one or more amino acid substitutions, deletions, insertions, or modifications compared to the naturally occurring subunit, so long as the subunits retain the ability to form TCRs conferring upon transfected T cells the ability to home to target cells, and participate in immunologically-relevant cytokine signaling. Engineered TCRs preferably also bind target cells displaying relevant tumor-associated peptides with high avidity, and optionally mediate efficient killing of target cells presenting the relevant peptide in vivo.

The nucleic acids encoding engineered TCRs may be preferably isolated from their natural context in a (naturally-occurring) chromosome of a T cell, and can be incorporated into suitable vectors as described herein. Both the nucleic acids and the vectors comprising them usefully can be transferred into a cell, which cell may be preferably T cells, more preferably γδ T cells. The modified T cells may be then able to express both chains of a TCR encoded by the transduced nucleic acid or nucleic acids. In preferred embodiments, engineered TCR may be an exogenous TCR because it is introduced into T cells that do not normally express the particular TCR. The essential aspect of the engineered TCRs is that it may have high avidity for a tumor antigen presented by a major histocompatibility complex (MHC) or similar immunological component. In contrast to engineered TCRs, CARs may be engineered to bind target antigens in an MHC independent manner.

In an aspect, engineered TCRs may function in γδ T cells in a CD8 (CD8αβ heterodimer and/or CD8αα homodimer)-independent manner. In another aspect, engineered TCRs may function in γδ T cells in a CD8 (CD8αβ heterodimer and/or CD8αα homodimer)-dependent manner. In the latter case, γδ T cells may be modified by expressing exogenous nucleic acids encoding both TCR and CD8 (CD8α and CD8β chains or CD8α chain). In an aspect, γδ T cells may be transduced or transfected with nucleic acids encoding TCR and CD8 (CD8α and CD8β chains or CD8α chain), which may reside on the same vector or on separate vectors.

The protein encoded by nucleic acids can be expressed with additional polypeptides attached to the amino-terminal or carboxyl-terminal portion of α-chain or β-chain of a TCR so long as the attached additional polypeptide does not interfere with the ability of α-chain or β-chain to form a functional T cell receptor and the MHC dependent antigen recognition.

Antigens that are recognized by the engineered TCRs may include, but are not limited to cancer antigens, including antigens on both hematological cancers and solid tumors. Illustrative antigens include, but are not limited to alpha folate receptor, 5T4, αvβ6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvlll, EGP2, EGP40, EPCAM, EphA2, EpCAM, FAP, fetal AchR, FRa, GD2, GD3, *Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, and VEGFR2.

In an aspect, T cells of the present disclosure may express a TCRs and antigen binding proteins described in U.S. Patent Application Publication No. 2017/0267738; U.S. Patent Application Publication No. 2017/0312350; U.S. Patent Application Publication No. 2018/0051080; U.S. Patent Application Publication No. 2018/0164315; U.S. Patent Application Publication No. 2018/0161396; U.S. Patent Application Publication No. 2018/0162922; U.S. Patent Application Publication No. 2018/0273602; U.S. Patent Application Publication No. 2019/0016801; U.S. Patent Application Publication No. 2019/0002556; U.S. Patent Application Publication No. 2019/0135914; U.S. Pat. No. 10,538,573; U.S. Pat. No. 10,626,160; U.S. Patent Application Publication No. 2019/0321478; U.S. Patent Application Publication No. 2019/0256572; U.S. Pat. No. 10,550,182; U.S. Pat. No. 10,526,407; U.S. Patent Application Publication No. 2019/0284276; U.S. Patent Application Publication No. 2019/0016802; U.S. Patent Application Publication No. 2019/0016803; U.S. Patent Application Publication No. 2019/0016804; U.S. Pat. No. 10,583,573; U.S. Patent Application Publication No. 2020/0339652; U.S. Pat. No. 10,537,624; U.S. Pat. No. 10,596,242; U.S. Patent Application Publication No. 2020/0188497; U.S. Pat. No. 10,800,845; U.S. Patent Application Publication No. 2020/0385468; U.S. Pat. No. 10,527,623; U.S. Pat. No. 10,725,044; U.S. Patent Application Publication No. 2020/0249233; U.S. Pat. No. 10,702,609; U.S. Patent Application Publication No. 2020/0254106; U.S. Pat. No. 10,800,832; U.S. Patent Application Publication No. 2020/0123221; U.S. Pat. No. 10,590,194; U.S. Pat. No. 10,723,796; U.S. Patent Application Publication No. 2020/0140540; U.S. Pat. No. 10,618,956; U.S. Patent Application Publication No. 2020/0207849; U.S. Patent Application Publication No. 2020/0088726; and U.S. Patent Application Publication No. 2020/0384028; the contents of each of these publications and sequence listings described therein are herein incorporated by reference in their entireties. T cells may be αβ T cells, γδ T cells, or natural killer T cells. In an embodiment, TCRs described herein may be single-chain TCRs or soluble TCRs.

### Chimeric Antigen Receptors (CARs)

T cell manufacturing methods disclosed herein may include modifying T cells to express one or more CARs. T cells may be αβ T cells, γδ T cells, or natural killer T cells. In various embodiments, the present disclosure provides T cells genetically engineered with vectors designed to express CARs that redirect cytotoxicity toward tumor cells. CARs are molecules that combine antibody-based specificity for a target antigen, e.g., tumor antigen, with a T cell receptor-activating intracellular domain to generate a chimeric protein that exhibits a specific anti-tumor cellular immune activity. As used herein, the term, “chimeric,” describes being composed of parts of different proteins or DNAs from different origins.

CARs may contain an extracellular domain that binds to a specific target antigen (also referred to as a binding domain or antigen-specific binding domain), a transmembrane domain and an intracellular signaling domain. The main characteristic of CARs may be their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific coreceptors.

In particular embodiments, CARs may contain an extracellular binding domain including but not limited to an antibody or antigen binding fragment thereof, a tethered ligand, or the extracellular domain of a coreceptor, that specifically binds a target antigen that is a tumor-associated antigen (TAA) or a tumor-specific antigen (TSA). In certain embodiments, the TAA or TSA may be expressed on a blood cancer cell. In another embodiment, the TAA or TSA may be expressed on a cell of a solid tumor. In particular embodiments, the solid tumor may be a glioblastoma, a non-small cell lung cancer, a lung cancer other than a non-small cell lung cancer, breast cancer, prostate cancer, pancreatic cancer, liver cancer, colon cancer, stomach cancer, a cancer of the spleen, skin cancer, a brain cancer other than a glioblastoma, a kidney cancer, a thyroid cancer, or the like.

In particular embodiments, the TAA or TSA may be selected from the group consisting of alpha folate receptor, 5T4, ανβ6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRa, GD2, GD3, *Glypican-3(GPC3), HLA-A1+MAGE1, HLA-A2+MAGE1, HLA-A3+MAGE1, HLA-A1+NY-ESO-1, HLA-A2+NY-ESO-1 HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, and VEGFR2.

In an aspect, tumor associated antigen (TAA) peptides that are capable of use with the methods and embodiments described herein include, for example, those TAA peptides described in U.S. Publication 20160187351, U.S. Publication 20170165335, U.S. Publication 20170035807, U.S. Publication 20160280759, U.S. Publication 20160287687, U.S. Publication 20160346371, U.S. Publication 20160368965, U.S. Publication 20170022251, U.S. Publication 20170002055, U.S. Publication 20170029486, U.S. Publication 20170037089, U.S. Publication 20170136108, U.S. Publication 20170101473, U.S. Publication 20170096461, U.S. Publication 20170165337, U.S. Publication 20170189505, U.S. Publication 20170173132, U.S. Publication 20170296640, U.S. Publication 20170253633, U.S. Publication 20170260249, U.S. Publication 20180051080, and U.S. Publication No. 20180164315, the contents of each of these publications and sequence listings described therein are herein incorporated by reference in their entireties.

In an aspect, T cells described herein selectively recognize cells which present a TAA peptide described in one of more of the patents and publications described above.

In another aspect, TAA that are capable of use with the methods and embodiments described herein include at least one selected from SEQ ID NO: 6 to SEQ ID NO: 166. In an aspect, T cells selectively recognize cells which present a TAA peptide described in SEQ ID NO: 6-166 or any of the patents or applications described herein.

| SEQ | Amino Acid |
|---|---|
| ID NO: | Sequence |
| 6 | YLYDSETKNA |
| 7 | HLMDQPLSV |
| 8 | GLLKKINSV |
| 9 | FLVDGSSAL |
| 10 | FLFDGSANLV |
| 11 | FLYKIIDEL |
| 12 | FILDSAETTTL |
| 13 | SVDVSPPKV |
| 14 | VADKIHSV |
| 15 | IVDDLTINL |
| 16 | GLLEELVTV |
| 17 | TLDGAAVNQV |
| 18 | SVLEKEIYSI |
| 19 | LLDPKTIFL |
| 20 | YTFSGDVQL |
| 21 | YLMDDFSSL |
| 22 | KVWSDVTPL |
| 23 | LLWGHPRVALA |
| 24 | KIWEELSVLEV |
| 25 | LLIPFTIFM |
| 26 | FLIENLLAA |
| 27 | LLWGHPRVALA |
| 28 | FLLEREQLL |
| 29 | SLAETIFIV |
| 30 | TLLEGISRA |
| 31 | ILQDGQFLV |
| 32 | VIFEGEPMYL |
| 33 | SLFESLEYL |
| 34 | SLLNQPKAV |
| 35 | GLAEFQENV |
| 36 | KLLAVIHEL |
| 37 | TLHDQVHLL |
| 38 | TLYNPERTITV |
| 39 | KLQEKIQEL |
| 40 | SVLEKEIYSI |
| 41 | RVIDDSLVVGV |
| 42 | VLFGELPAL |
| 43 | GLVDIMVHL |
| 44 | FLNAIETAL |
| 45 | ALLQALMEL |
| 46 | ALSSSQAEV |
| 47 | SLITGQDLLSV |
| 48 | QLIEKNWLL |
| 49 | LLDPKTIFL |
| 50 | RLHDENILL |
| 51 | YTFSGDVQL |
| 52 | GLPSATTTV |
| 53 | GLLPSAESIKL |
| 54 | KTASINQNV |
| 55 | SLLQHLIGL |
| 56 | YLMDDFSSL |
| 57 | LMYPYIYHV |
| 58 | KVWSDVTPL |
| 59 | LLWGHPRVALA |
| 60 | VLDGKVAVV |
| 61 | GLLGKVTSV |
| 62 | KMISAIPTL |
| 63 | GLLETTGLLAT |
| 64 | TLNTLDINL |
| 65 | VIIKGLEEI |
| 66 | YLEDGFAYV |
| 67 | KIWEELSVLEV |
| 68 | LLIPFTIFM |
| 69 | ISLDEVAVSL |
| 70 | KISDFGLATV |
| 71 | KLIGNIHGNEV |
| 72 | ILLSVLHQL |
| 73 | LDSEALLTL |
| 74 | VLQENSSDYQSNL |
| 75 | HLLGEGAFAQV |
| 76 | SLVENIHVL |
| 77 | YTFSGDVQL |
| 78 | SLSEKSPEV |
| 79 | AMFPDTIPRV |
| 80 | FLIENLLAA |
| 81 | FTAEFLEKV |
| 82 | ALYGNVQQV |
| 83 | LFQSRIAGV |
| 84 | ILAEEPIYIRV |
| 85 | FLLEREQLL |
| 86 | LLLPLELSLA |
| 87 | SLAETIFIV |
| 88 | AILNVDEKNQV |
| 89 | RLFEEVLGV |
| 90 | YLDEVAFML |
| 91 | KLIDEDEPLFL |
| 92 | KLFEKSTGL |
| 93 | SLLEVNEASSV |
| 94 | GVYDGREHTV |
| 95 | GLYPVTLVGV |
| 96 | ALLSSVAEA |
| 97 | TLLEGISRA |
| 98 | SLIEESEEL |
| 99 | ALYVQAPTV |
| 100 | KLIYKDLVSV |
| 101 | ILQDGQFLV |
| 102 | SLLDYEVSI |
| 103 | LLGDSSFFL |
| 104 | VIFEGEPMYL |
| 105 | ALSYILPYL |
| 106 | FLFVDPELV |
| 107 | SEWGSPHAAVP |
| 108 | ALSELERVL |
| 109 | SLFESLEYL |
| 110 | KVLEYVIKV |
| 111 | VLLNEILEQV |
| 112 | SLLNQPKAV |
| 113 | KMSELQTYV |
| 114 | ALLEQTGDMSL |
| 115 | VIIKGLEEITV |
| 116 | KQFEGTVEI |
| 117 | KLQEEIPVL |
| 118 | GLAEFQENV |
| 119 | NVAEIVIHI |
| 120 | ALAGIVTNV |
| 121 | NLLIDDKGTIKL |
| 122 | VLMQDSRLYL |
| 123 | KVLEHVVRV |
| 124 | LLWGNLPEI |
| 125 | SLMEKNQSL |
| 126 | KLLAVIHEL |
| 127 | ALGDKFLLRV |
| 128 | FLMKNSDLYGA |
| 129 | KLIDHQGLYL |
| 130 | GPGIFPPPPPQP |
| 131 | ALNESLVEC |
| 132 | GLAALAVHL |
| 133 | LLLEAVWHL |
| 134 | SIIEYLPTL |
| 135 | TLHDQVHLL |
| 136 | SLLMWITQC |
| 137 | FLLDKPQDLSI |
| 138 | YLLDMPLWYL |
| 139 | GLLDCPIFL |
| 140 | VLIEYNFSI |
| 141 | TLYNPERTITV |
| 142 | AVPPPPSSV |
| 143 | KLQEELNKV |
| 144 | KLMDPGSLPPL |
| 145 | ALIVSLPYL |
| 146 | FLLDGSANV |
| 147 | ALDPSGNQLI |
| 148 | ILIKHLVKV |
| 149 | VLLDTILQL |
| 150 | HLIAEIHTA |
| 151 | SMNGGVFAV |
| 152 | MLAEKLLQA |
| 153 | YMLDIFHEV |
| 154 | ALWLPTDSATV |
| 155 | GLASRILDA |
| 156 | ALSVLRLAL |
| 157 | SYVKVLHHL |
| 158 | VYLPKIPSW |
| 159 | NYEDHFPLL |
| 160 | VYIAELEKI |
| 161 | VHFEDTGKTLLF |
| 162 | VLSPFILTL |
| 163 | HLLEGSVGV |
| 164 | ALREEEEGV |
| 165 | KEADPTGHSY |
| 166 | TLDEKVAEL |

### Binding Domains of CARS

In particular embodiments, CARs contemplated herein comprise an extracellular binding domain that specifically binds to a target polypeptide, e.g., target antigen, expressed on tumor cell. As used herein, the terms, “binding domain,” “extracellular domain,”

“extracellular binding domain,” “antigen-specific binding domain,” and “extracellular antigen specific binding domain,” may be used interchangeably and provide a CAR with the ability to specifically bind to the target antigen of interest. A binding domain may include any protein, polypeptide, oligopeptide, or peptide that possesses the ability to specifically recognize and bind to a biological molecule (e.g., a cell surface receptor or tumor protein, lipid, polysaccharide, or other cell surface target molecule, or component thereof). A binding domain may include any naturally occurring, synthetic, semi-synthetic, or recombinantly produced binding partner for a biological molecule of interest.

In particular embodiments, the extracellular binding domain of a CAR may include an antibody or antigen binding fragment thereof. An “antibody” refers to a binding agent that is a polypeptide containing at least a light chain or heavy chain immunoglobulin variable region, which specifically recognizes and binds an epitope of a target antigen, such as a peptide, lipid, polysaccharide, or nucleic acid containing an antigenic determinant, such as those recognized by an immune cell. Antibodies may include antigen binding fragments thereof. The term may also include genetically engineered forms, such as chimeric antibodies (for example, humanized murine antibodies), hetero-conjugate antibodies, e.g., bispecific antibodies, and antigen binding fragments thereof. See also, Pierce Catalog and Handbook, 1994-1995 (Pierce Chemical Co., Rockford, IL); Kuby, J., Immunology, 3rd Ed., W. H. Freeman & Co., New York, 1997.

In particular embodiments, the target antigen may be an epitope of an alpha folate receptor, 5T4, ανβ6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, *Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2 polypeptide.

Light and heavy chain variable regions may contain a “framework” region interrupted by three hypervariable regions, also called “complementarity-determining regions” or “CDRs.” The CDRs can be defined or identified by conventional methods, such as by sequence according to Kabat et al (Wu, T T and Kabat, E. A., J Exp Med. 132 (2): 211-50, (1970); Borden, P. and Kabat E. A., PNAS, 84:2440-2443 (1987); (see, Kabat et al, Sequences of Proteins of Immunological Interest, U.S. Department of Health and Human Services, 1991, which is hereby incorporated by reference), or by structure according to Chothia et al (Choithia, C. and Lesk, A. M., J Mol. Biol, 196 (4): 901-917 (1987), Choithia, C. et al, Nature, 342:877-883(1989)). The contents of the afore-mentioned references are hereby incorporated by reference in their entireties. The sequences of the framework regions of different light or heavy chains may be relatively conserved within a species, such as humans. The framework region of an antibody that is the combined framework regions of the constituent light and heavy chains may serve to position and align the CDRs in three-dimensional space. The CDRs may be primarily responsible for binding to an epitope of an antigen. The CDRs of each chain may be typically referred to as CDR1, CDR2, and CDR3, numbered sequentially starting from the N-terminus, and may be also typically identified by the chain, in which the particular CDR is located. Thus, the CDRs located in the variable domain of the heavy chain of the antibody may be referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) may have different CDRs. Although it is the CDRs that vary from antibody to antibody, only a limited number of amino acid positions within the CDRs are directly involved in antigen binding. These positions within the CDRs are called specificity determining residues (SDRs).

References to “VH” or “VH” refers to the variable region of an immunoglobulin heavy chain, including that of an antibody, Fv, scFv, dsFv, Fab, or other antibody fragment. References to “VL” or “VL” refers to the variable region of an immunoglobulin light chain, including that of an antibody, Fv, scFv, dsFv, Fab, or other antibody fragment.

A “monoclonal antibody” is an antibody produced by a single clone of B lymphocytes or by a cell into which the light and heavy chain genes of a single antibody have been transfected. Monoclonal antibodies may be produced by methods known to those of skill in the art, for example, by making hybrid antibody-forming cells from a fusion of myeloma cells with immune spleen cells. Monoclonal antibodies may include humanized monoclonal antibodies.

A “chimeric antibody” has framework residues from one species, such as human, and CDRs (which generally confer antigen binding) from another species, such as a mouse. In particular preferred embodiments, a CAR disclosed herein may contain antigen-specific binding domain that is a chimeric antibody or antigen binding fragment thereof.

In certain embodiments, the antibody may be a humanized antibody (such as a humanized monoclonal antibody) that specifically binds to a surface protein on a tumor cell. A “humanized” antibody is an immunoglobulin including a human framework region and one or more CDRs from a non-human (for example a mouse, rat, or synthetic) immunoglobulin. Humanized antibodies can be constructed by means of genetic engineering (see for example, U.S. Pat. No. 5,585,089, the content of which is hereby incorporated by reference in its entirety).

In embodiments, the extracellular binding domain of a CAR may contain an antibody or antigen binding fragment thereof, including but not limited to a Camel Ig (a camelid antibody (VHH)), Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, Nanobody).

“Camel Ig” or “camelid VHH” as used herein refers to the smallest known antigen-binding unit of a heavy chain antibody (Koch-Nolte, et al, FASEB J., 21:3490-3498 (2007), the content of which is hereby incorporated by reference in its entirety). A “heavy chain antibody” or a “camelid antibody” refers to an antibody that contains two VH domains and no light chains (Riechmann L. et al, J. Immunol. Methods 231:25-38 (1999); WO94/04678; W094/25591; U.S. Pat. No. 6,005,079; the contents of which are hereby incorporated by reference in its entirety).

“IgNAR” of “immunoglobulin new antigen receptor” refers to class of antibodies from the shark immune repertoire that consist of homodimers of one variable new antigen receptor (VNAR) domain and five constant new antigen receptor (CNAR) domains.

Papain digestion of antibodies produces two identical antigen-binding fragments, called “Fab” fragments, each with a single antigen-binding site, and a residual “Fc” fragment, whose name reflects its ability to crystallize readily. The Fab fragment contains the heavy-and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” is the minimum antibody fragment which contains a complete antigen-binding site. In a single-chain Fv (scFv) species, one heavy-and one light-chain variable domain can be covalently linked by a flexible peptide linker such that the light and heavy chains can associate in a “dimeric” structure analogous to that in a two-chain Fv species.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al, Nat. Med. 9:129-134 (2003); and Hollinger et al, PNAS USA 90:6444-6448 (1993). Triabodies and tetrabodies are also described in Hudson et al, Nat. Med. 9:129-134 (2003). The contents of the afore-mentioned references are hereby incorporated by reference in their entireties.

“Single domain antibody” or “sdAb” or “nanobody” refers to an antibody fragment that consists of the variable region of an antibody heavy chain (VH domain) or the variable region of an antibody light chain (VL domain) (Holt, L., et al, Trends in Biotechnology, 21 (11): 484-490, the content of which is hereby incorporated by reference in its entirety).

“Single-chain Fv” or “scFv” antibody fragments comprise the VH and VL domains of antibody, wherein these domains are present in a single polypeptide chain and in either orientation {e.g., VL-VH or VH-VL). Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315, the content of which is hereby incorporated by reference in its entirety.

In a certain embodiment, the scFv binds an alpha folate receptor, 5T4, ανβ6 integrin, BCMA, B7-H3, B7-H6, CALX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRa, GD2, GD3, *Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2 polypeptide.

### Linkers of CARS

In certain embodiments, the CARs may contain linker residues between the various domains, e.g., between VH and VL domains, added for appropriate spacing and conformation of the molecule. CARs may contain one, two, three, four, or five or more linkers. In particular embodiments, the length of a linker may be about 1 to about 25 amino acids, about 5 to about 20 amino acids, or about 10 to about 20 amino acids, or any intervening length of amino acids. In some embodiments, the linker may be 1, 2, 3, 4, 5, 6, 7, 8, 9, 10, 11, 12, 13, 14, 15, 16, 17, 18, 19, 20, 21, 22, 23, 24, 25, or more amino acids long. Illustrative examples of linkers include glycine polymers (G) n; glycine-serine polymers (Gi_sSi_5) n, where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains of fusion proteins, such as CARs. Glycine may access significantly more phi-psi space than even alanine, and may be much less restricted than residues with longer side chains (see Scheraga, Rev. Computational Chem. 11173-142 (1992), the content of which is hereby incorporated by reference in its entirety). The ordinarily skilled artisan may recognize that design of a CAR in particular embodiments can include linkers that may be all or partially flexible, such that the linker can include a flexible linker as well as one or more portions that confer less flexible structure to provide for a desired CAR structure.

In particular embodiments a CAR may include a scFV that may further contain a variable region linking sequence. A “variable region linking sequence,” is an amino acid sequence that connects a heavy chain variable region to a light chain variable region and provides a spacer function compatible with interaction of the two sub-binding domains so that the resulting polypeptide retains a specific binding affinity to the same target molecule as an antibody that may contain the same light and heavy chain variable regions. In one embodiment, the variable region linking sequence may be 1, 2, 3, 4, 5, 6, 7, 8, 9, 10, 11, 12, 13, 14, 15, 16, 17, 18, 19, 20, 21, 22, 23, 24, 25, or more amino acids long. In a particular embodiment, the variable region linking sequence may contain a glycine-serine polymer (Gi_sSi_5)n, where n is an integer of at least 1, 2, 3, 4, or 5. In another embodiment, the variable region linking sequence comprises a (G₄S)₃amino acid linker.

### Spacer Domains of CARS

In particular embodiments, the binding domain of the CAR may be followed by one or more “spacer domains,” which refers to the region that moves the antigen binding domain away from the effector cell surface to enable proper cell/cell contact, antigen binding and activation (Patel et al, Gene Therapy, 1999; 6:412-419, the content of which is hereby incorporated by reference in its entirety). The spacer domain may be derived either from a natural, synthetic, semi-synthetic, or recombinant source. In certain embodiments, a spacer domain may be a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region. In one embodiment, the spacer domain may include the CH2 and CH3 of IgG1.

### Hinge Domains of CARS

The binding domain of CAR may be generally followed by one or more “hinge domains,” which may μlay a role in positioning the antigen binding domain away from the effector cell surface to enable proper cell/cell contact, antigen binding and activation. CAR generally may include one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs may include the hinge region derived from the extracellular regions of type 1 membrane proteins, such as CD8a, CD4, CD28 and CD7, which may be wild-type hinge regions from these molecules or may be altered. In another embodiment, the hinge domain may include a CD8α hinge region.

### Transmembrane (TM) Domains of CARS

The “transmembrane domain” may be the portion of CAR that can fuse the extracellular binding portion and intracellular signaling domain and anchors CAR to the μlasma membrane of the immune effector cell. The TM domain may be derived either from a natural, synthetic, semi-synthetic, or recombinant source. Illustrative TM domains may be derived from (including at least the transmembrane region(s) of) the α, β, or ζ chain of the T-cell receptor, CD38, CD37, CD4, CD5, CD9, CD16, CD22, CD27, CD28, CD33, CD37, CD45, CD64, CD80, CD86, CD 134, CD137, and CD154. In one embodiment, CARs may contain a TM domain derived from CD8a. In another embodiment, a CAR contemplated herein comprises a TM domain derived from CD8α and a short oligo-or polypeptide linker, preferably between 1, 2, 3, 4, 5, 6, 7, 8, 9, or 10 amino acids in length that links the TM domain and the intracellular signaling domain of CAR. A glycine-serine linker provides a particularly suitable linker.

### Intracellular Signaling Domains of CARS

In particular embodiments, CARs may contain an intracellular signaling domain. An “intracellular signaling domain,” refers to the part of a CAR that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

The term “effector function” refers to a specialized function of the cell. Effector function of the T cell, for example, may be cytolytic activity or help or activity including the secretion of a cytokine. Thus, the term “intracellular signaling domain” refers to the portion of a protein, which can transduce the effector function signal and that direct the cell to perform a specialized function. While usually the entire intracellular signaling domain can be employed, in many cases it is not necessary to use the entire domain. To the extent that a truncated portion of an intracellular signaling domain may be used, such truncated portion may be used in μlace of the entire domain as long as it can transduce the effector function signal. The term intracellular signaling domain may be meant to include any truncated portion of the intracellular signaling domain sufficient to transducing effector function signal.

It is known that signals generated through TCR alone are insufficient for full activation of the T cell and that a secondary or costimulatory signal may be also required. Thus, T cell activation can be said to be mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. In preferred embodiments, CAR may include an intracellular signaling domain that may contain one or more “costimulatory signaling domain” and a “primary signaling domain.” Primary signaling domains can regulate primary activation of the TCR complex either in a stimulatory way, or in an inhibitory way. Primary signaling domains that act in a stimulatory manner may contain signaling motifs, which are known as immunoreceptor tyrosine-based activation motifs or ITAMs. Illustrative examples of ITAM containing primary signaling domains that are of particular use in the invention may include those derived from TCRζ, FcRγ, FcRβ, CD3γ, CD3δ, CD3ϵ, CD3ζ CD22, CD79a, CD79b, and CD66d. In particular preferred embodiments, CAR may include a CD37 primary signaling domain and one or more costimulatory signaling domains. The intracellular primary signaling and costimulatory signaling domains may be linked in any order in tandem to the carboxyl terminus of the transmembrane domain.

CARs may contain one or more costimulatory signaling domains to enhance the efficacy and expansion of T cells expressing CAR receptors. As used herein, the term, “costimulatory signaling domain,” or “costimulatory domain”, refers to an intracellular signaling domain of a costimulatory molecule. Illustrative examples of such costimulatory molecules may include CD27, CD28, 4-1BB (CD137), OX40 (CD134), CD30, CD40, PD-1, ICOS (CD278), CTLA4, LFA-1, CD2, CD7, LIGHT, TRIM, LCK3, SLAM, DAP10, LAG3, HVEM and NKD2C, and CD83. In one embodiment, CAR may contain one or more costimulatory signaling domains selected from the group consisting of CD28, CD137, and CD134, and a CD37 primary signaling domain.

In one embodiment, CAR may contain an scFv that binds an alpha folate receptor, 5T4, avB6integrin, BCMA, B7-H3, B7-H6, CALX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRa, GD2, GD3, *Glypican-3 (GPC3), HLA-A1+MAGE1, HLA-A2+M AGE1, HLA-A3+MAGE1, HLA-AI+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2 polypeptide; a transmembrane domain derived from a polypeptide selected from the group consisting of: CD8α; CD4, CD45, PD1, and CD152; and one or more intracellular costimulatory signaling domains selected from the group consisting of: CD28, CD54, CD134, CD137, CD152, CD273, CD274, and CD278; and a CD3ζ primary signaling domain.

In another embodiment, CAR may contain an scFv that binds an alpha folate receptor, 5T4, ανβ6 integrin, BCMA, B7-H3, B7-H6, CALX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRa, GD2, GD3, *Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2 polypeptide; a hinge domain selected from the group consisting of: IgG1 hinge/CH2/CH3 and CD8a, and CD8a; a transmembrane domain derived from a polypeptide selected from the group consisting of: CD8a; CD4, CD45, PD1, and CD152; and one or more intracellular costimulatory signaling domains selected from the group consisting of: CD28, CD 134, and CD 137; and a CD37 primary signaling domain.

In yet another embodiment, CAR may contain an scFv, further including a linker, that binds an alpha folate receptor, 5T4, ανβ6 integrin, BCMA, B7-H3, B7-H6, CAIX, CD 19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, *Glypican-3(GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2 polypeptide; a hinge domain selected from the group consisting of: IgG1 hinge/CH2/CH3 and CD8α, and CD8α; a transmembrane domain comprising a TM domain derived from a polypeptide selected from the group consisting of: CD8a; CD4, CD45, PD1, and CD 152, and a short oligo-or polypeptide linker, preferably between 1, 2, 3, 4, 5, 6, 7, 8, 9, or 10 amino acids in length that links the TM domain to the intracellular signaling domain of the CAR; and one or more intracellular costimulatory signaling domains selected from the group consisting of: CD28, CD 134, and CD137; and a CD3ζ primary signaling domain.

In a particular embodiment, CAR may contain an scFv that binds an alpha folate receptor, 5T4, ανβ6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRa, GD2, GD3, *Glypican-3 (GPC3), HLA-A1+MAGE1, HLA-A2+M AGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2 polypeptide; a hinge domain containing a CD8α polypeptide; a CD8a transmembrane domain containing a polypeptide linker of about 3 amino acids; one or more intracellular costimulatory signaling domains selected from the group consisting of: CD28, CD134, and CD137; and a CD3ζ primary signaling domain.

### Viruses

In an aspect, “viruses” refers to natural occurring viruses as well as artificial viruses. Viruses in accordance with some embodiments of the present disclosure may be either an enveloped or non-enveloped virus. Parvoviruses (such as AAVs) are examples of non-enveloped viruses. In a preferred embodiment, the viruses may be enveloped viruses. In preferred embodiments, the viruses may be retroviruses and in particular lentiviruses. Viral envelope proteins that can promote viral infection of eukaryotic cells may include HIV-1 derived lentiviral vectors (LVs) pseudotyped with envelope glycoproteins (GPs) from the vesicular stomatitis virus (VSV-G), the modified feline endogenous retrovirus (RD114TR), and the modified gibbon ape leukemia virus (GALVTR). These envelope proteins can efficiently promote entry of other viruses, such as parvoviruses, including adeno-associated viruses (AAV), thereby demonstrating their broad efficiency. For example, other viral envelop proteins may be used including Moloney murine leukemia virus (MLV) 4070 env (such as described in Merten et al., J. Virol. 79:834-840, 2005; which is incorporated herein by reference), RD114 env (SEQ ID NO: 2), chimeric envelope protein RD114pro or RDpro (which is an RD114-HIV chimera that was constructed by replacing the R peptide cleavage sequence of RD114 with the HIV-1 matrix/capsid (MA/CA) cleavage sequence, such as described in Bell et al. Experimental Biology and Medicine 2010; 235:1269-1276; which is incorporated herein by reference), baculovirus GP64 env (such as described in Wang et al. J. Virol. 81:10869-10878, 2007; which is incorporated herein by reference), or GALV env (such as described in Merten et al., J. Virol. 79:834-840, 2005; which is incorporated herein by reference), or derivatives thereof.

### RD114TR

RD114TR is a chimeric envelope glycoprotein made of the extracellular and transmembrane domains of the feline leukemia virus RD114 and the cytoplasmic tail (TR) of the amphotropic murine leukemia virus envelope. RD114TR pseudotyped vectors can mediate efficient gene transfer into human hematopoietic progenitors and NOD/SCID repopulating cells. Di Nunzio et al., Hum. Gene Ther: 811-820 (2007)), the contents of which are incorporated by reference in their entirety. RD114 pseudotyped vectors can also mediate efficient gene transfer in large animal models. (Neff et al., Mal. Ther. 2:157-159 (2004); Hu et al., Mal. Ther: 611-617 (2003); and Kelly et al., Blood Cells, Molecules, & Diseases 30:132-143 (2003)), the contents of each of these references are incorporated by reference in their entirety.

The present disclosure may include RD114TR variants having at least about 50%, at least about 60%, at least about 70%, at least about 80%, at least about 90%, at least about 95%, at least about 98%, at least about 99%, or 100% sequence identity to the amino acid sequence of SEQ ID NO: 1 or SEQ ID NO: 5. For example, an RD114TR variant (RD114TRv1 (SEQ ID NO: 5)) having at least about 95%, at least about 96%, at least about 97%, at least about 98%, or at least about 99% sequence identity to RD114TR (SEQ ID NO: 1) may be used. In an aspect, the disclosure provides for RD114TR variants having modified amino acid residues. A modified amino acid residue may be selected from an amino acid insertion, deletion, or substitution. In an aspect, a substitution described herein is a conservative amino acid substitution. That is, amino acids of RD114TR may be replaced by other amino acids having similar properties (conservative changes, such as similar hydrophobicity, hydrophilicity, antigenicity, propensity to form or break a-helical structures or 3-sheet structures). In an aspect, RD114TR may have 1, 2, 3, 4, 5, 6, 7, 8, 9, or 10 amino acid modification(s). In another aspect, RD114TR may have at most 1, 2, 3, 4, 5, 6, 7, 8, 9, or 10 amino acid modification(s). In yet another aspect, RD114TR may have at least 1, 2, 3, 4, 5, 6, 7, 8, 9, or 10 amino acid modification(s). Non-limiting examples of conservative substitutions may be found in, for example, Creighton (1984) Proteins. W.H. Freeman and Company, the contents of which are incorporated by reference in their entirety.

In another aspect, the present disclosure may include variants having at least about 50%, at least about 60%, at least about 70%, at least about 80%, at least about 90%, at least about 95%, at least about 98%, at least about 99%, or 100% sequence identity to the amino acid sequence of SEQ ID NO: 1, 2, 3, 4, or 5.

In an aspect, conservative substitutions may include those, which are described by Dayhoff in “The Atlas of Protein Sequence and Structure. Vol. 5”, Natl. Biomedical Research, the contents of which are incorporated by reference in their entirety. For example, in an aspect, amino acids, which belong to one of the following groups, can be exchanged for one another, thus, constituting a conservative exchange: Group 1: alanine (A), proline (P), glycine (G), asparagine (N), serine(S), threonine (T); Group 2: cysteine (C), serine (S), tyrosine (Y), threonine (T); Group 3: valine (V), isoleucine (I), leucine (L), methionine (M), alanine (A), phenylalanine (F); Group 4: lysine (K), arginine (R), histidine (H); Group 5: phenylalanine (F), tyrosine (Y), tryptophan (W), histidine (H); and Group 6: aspartic acid (D), glutamic acid (E).

In an aspect, conservative amino acid substitution may include the substitution of an amino acid by another one of the same class, for example, (1) nonpolar: Ala, Val, Leu, Ile, Pro, Met, Phe, Trp; (2) uncharged polar: Gly, Ser, Thr, Cys, Tyr, Asn, Gln; (3) acidic: Asp, Glu; and (4) basic: Lys, Arg, His. Other conservative amino acid substitutions may also be made as follows: (1) aromatic: Phe, Tyr, His; (2) proton donor: Asn, Gln, Lys, Arg, His, Trp; and (3) proton acceptor: Glu, Asp, Thr, Ser, Tyr, Asn, Gln (see, U.S. Pat. No. 10,106,805).

In another aspect, conservative substitutions may be made in accordance with Table A. Methods for predicting tolerance to protein modification may be found in, for example, Guo et al., Proc. Natl. Acad. Sci., USA, 101 (25): 9205-9210 (2004), the contents of which are incorporated by reference in their entirety.

Conservative Amino Acid Substitutions
Table A

| Amino Acid | Substitutions (others are known in the art) |
|---|---|
| Ala | Ser, Gly, Cys |
| Arg | Lys, Gln, His |
| Asn | Gln, His, Glu, Asp |
| Asp | Glu, Asn, Gln |
| Cys | Ser, Met, Thr |
| Gln | Asn, Lys, Glu, Asp, Arg |
| Glu | Asp, Asn, Gln |
| Gly | Pro, Ala, Ser |
| His | Asn, Gln, Lys |
| Ile | Leu, Val, Met, Ala |
| Leu | Ile, Val, Met, Ala |
| Lys | Arg, Gln, His |
| Met | Leu, Ile, Val, Ala, Phe |
| Phe | Met, Leu, Tyr, Trp, His |
| Ser | Thr, Cys, Ala |
| Thr | Ser, Val, Ala |
| Trp | Tyr, Phe |
| Tyr | Trp, Phe, His |
| Val | Ile, Leu, Met, Ala, Thr |

In an aspect, transgene expression for RD114TR-pseudotyped retroviral vector at about 10-day post-transduction is about 20% to about 60% about 30% to about 50%, or about 35% to about 45%. In an aspect, transgene expression for RD114TR-pseudotyped retroviral vector at 10-day post-transduction is about 20% to about 60% about 30% to about 50%, or about 35% to about 45% relative to transgene expression for VSV-G-pseudotyped vectors at day 10 post-transduction of about 5% to about 25%, about 2% to about 20%, about 3% to about 15%, or about 5% to about 12% under the same conditions. In yet another aspect, transgene expression for RD114TR-pseudotyped retroviral vector at 10-day post-transduction is about 40% relative to transgene expression for VSV-G-pseudotyped vectors at day 10 post-transduction of about 3.6%.

In yet another aspect, transgene expression for RD114TR-pseudotyped retroviral vector at about 5-day post-transduction is about 20% to about 50% about 15% to about 30%, or about 20% to about 30%. In an aspect, transgene expression for RD114TR-pseudotyped retroviral vector at 5-day post-transduction is about 20% to about 50% about 15% to about 30%, or about 20% to about 30% relative to transgene expression for VSV-G-pseudotyped vectors at day 5 post-transduction of about 10% to about 20%, about 15% to about 25%, or about 17.5% to about 20% under the same conditions. In yet another aspect, transgene expression for RD114TR-pseudotyped retroviral vector at 5-day post-transduction is about 24% relative to transgene expression for VSV-G-pseudotyped vectors at day 5 post-transduction of about 19%.

In another aspect, transgene expression for RD114TR-pseudotyped retroviral vector at 10-day post-transduction is about 2 times, about 3 times, about 4 times, about 5 times, or about 10 times, about 11times, or about 12 times or more relative to transgene expression for VSV-G-pseudotyped vectors at day 10post-transduction.

In an aspect, the disclosure provides for methods of using retrovirus with RD114TR pseudotype (for example, SEQ ID NO: 1, SEQ ID NO: 5, or variants thereof) to transduce T cells. In another aspect, T cells are more efficiently transduced by retrovirus with RD114TR pseudotype (for example, SEQ ID NO: 1, SEQ ID NO: 5, or variants thereof) as compared to retrovirus with VSV-G pseudotype (for example, SEQ ID NO: 3). In another aspect, a RD114TR envelope is utilized to pseudotype a lentivector, which is then used to transduce T cells with excellent efficiency.

Engineered γδ T-cells may be generated with various methods. For example, a polynucleotide encoding an expression cassette that comprises a tumor recognition, or another type of recognition moiety, can be stably introduced into the γδ T-cell by a transposon/transposase system or a viral-based gene transfer system, such as a lentiviral or a retroviral system, or another suitable method, such as transfection, electroporation, transduction, lipofection, calcium phosphate (CaPO4), nanoengineered substances, such as Ormosil, viral delivery methods, including adenoviruses, retroviruses, lentiviruses, adeno-associated viruses, or another suitable method. A number of viral methods have been used for human gene therapy, such as the methods described in WO 1993020221, which is incorporated herein in its entirety. Non-limiting examples of viral methods that can be used to engineer γδ T cells may include y-retroviral, adenoviral, lentiviral, herpes simplex virus, vaccinia virus, pox virus, or adeno-virus associated viral methods.

FIG. 2 shows the activated T cells may be engineered by transducing with a viral vector, such as RD114TR γ-retroviral vector and RD114TR lentiviral vector, expressing exogenous genes of interest, such as αβ TCRs against specific cancer antigen and CD8, into isolated γδ T cells. Viral vectors may also contain post-transcriptional regulatory element (PRE), such as Woodchuck PRE (WPRE) to enhance the expression of the transgene by increasing both nuclear and cytoplasmic mRNA levels. One or more regulatory elements including mouse RNA transport element (RTE), the constitutive transport element (CTE) of the simian retrovirus type 1 (SRV-1), and the 5′ untranslated region of the human heat shock protein 70 (Hsp70 5′UTR) may also be used and/or in combination with WPRE to increase transgene expression. Transduction may be carried out once or multiple times to achieve stable transgene expression in small scale, e.g., 24 to 4-6 well plates, or mid/large scale for ½-5 days, e.g., 1 day.

RD114TR is a chimeric glycoprotein containing an extracellular and transmembrane domain of feline endogenous virus (RD114) fused to cytoplasmic tail (TR) of murine leukemia virus. In an aspect, transgene expression for RD114TR-pseudotyped retroviral vector at 10-day post-transduction is higher relative to VSV-G-pseudotyped vectors.

Other viral envelop proteins, such as VSV-G env, MLV 4070 env, RD114 env, chimeric envelope protein RD114pro, baculovirus GP64 env, or GALV env, or derivatives thereof, may also be used.

### Non-Viral Vectors

The vector is a non-viral vector, in that it is not based on a virus. It does not include any viral components in order for the vector to gain entry into the cell. A non-viral vector may be selected from plasmids, minicircles, comsids, artificial chromosomes (e.g., BAC), linear covalently closed (LCC) DNA vectors (e.g., minicircles, minivectors and miniknots), linear covalently closed (LCC) vectors (e.g., MIDGE, MiLV, ministering, miniplasmids), mini-intronic μlasmids, pDNA expression vectors, or nuclease-mediated genetic editing, e.g., zinc-finger nucleases (ZFNs), transcription activator-like effector nucleases (TALENs), and clustered regularly interspaced short palindromic repeats (CRISPR).

In some embodiments, the non-viral vector system for the delivery of nucleic acids may include a polymer conjugate consisting of polyethylene glycol (PEG), polyethylenimine (PEI), and peptide sequences with PTD/CPP-functionality. For example, a protein with PTD/CPP-functionality may be TAT-peptide or a peptide sequence, which may be related to TAT-peptide. For example, a sequence related to the TAT-peptide may be a decapeptide sequence GRKKKRRQRC (SEQ ID NO: 167). Other well-known TAT-peptide related sequences can be used alternatively. In addition to the stability with respect to intracellular enzymes (e.g. in endosomes, lysosomes), the non-viral vector system for the delivery of nucleic acid according to the present application may also be very stable in an extracellular environment. For example, as compared to PEI, the stability of TAT-PEG-PEI-polyplexes may be significantly higher in the presence of high concentrations of heparin, Alveofact®, BALF, and DNase I.

In some embodiments, polypeptides, e.g., TCRs and CARs, described herein can also be introduced into effector cells, such as T cells, using non-viral based delivery systems, such as the “Sleeping Beauty (SB) Transposon System,” which refers a synthetic DNA transposon system to introduce DNA sequences into the chromosomes of vertebrates. The system is described, for example, in U.S. Pat. Nos. 6,489,458 and 8,227,432. The contents of which are hereby incorporated by reference in their entireties.

The Sleeping Beauty transposon system may be composed of a Sleeping Beauty (SB) transposase and a SB transposon. DNA transposons translocate from one DNA site to another in a simple, cut-and-paste manner. Transposition may be a precise process, in which a defined DNA segment may be excised from one DNA molecule and moved to another site in the same or different DNA molecule or genome. As do other Tc1/mariner-type transposases, SB transposase inserts a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site may be duplicated in the process of transposon integration. This duplication of the TA sequence may be a hallmark of transposition and used to ascertain the mechanism in some experiments. The transposase can be encoded either within the transposon or the transposase can be supplied by another source, in which case the transposon becomes a non-autonomous element. Non-autonomous transposons may be useful as genetic tools because after insertion they cannot independently continue to excise and re-insert. SB transposons envisaged to be used as non-viral vectors for introduction of genes into genomes of vertebrate animals and for gene therapy.

Briefly, the Sleeping Beauty (SB) system (Hackett et al., Mol Ther 18:674-83, (2010)) was adapted to genetically modify the T cells (Cooper et al., Blood 105:1622-31, (2005)). This involved two steps: (i) the electro-transfer of DNA μlasmids expressing a SB transposon [i.e., chimeric antigen receptor (CAR) to redirect T-cell specificity (Jin et al., Gene Ther 18:849-56, (2011); Kebriaei et al., Hum Gene Ther 23:444-50, (2012)) and SB transposase and (ii) the propagation and expansion of T cells stably expressing integrants on designer artificial antigen-presenting cells (AaPC) derived from the K562 cell line (also known as AaPCs (Activating and Propagating Cells). The contents of the afore-cited references are hereby incorporated by reference in their entireties. In one embodiment, the SB transposon system may include coding sequence encoding mbIL-15, a cell tag and/or a CAR. In one embodiment, the SB transposon system may include coding sequence encoding mbIL-15, a cell tag and/or a TCR. In another embodiment, the second step (ii) is eliminated and the genetically modified T cells may be cryopreserved or immediately infused into a patient. In certain embodiments, the genetically modified T cells may be not cryopreserved before infusion into a patient. In some embodiments, the Sleeping Beauty transposase may be SB11, SB100X, or SB110.

The non-viral vector system for the delivery of nucleic acids according to the present application may be applied to the patient, as part of a pharmaceutically acceptable composition, either by inhalation, orally, rectally, parental intravenously, intramuscularly or subcutaneously, intra-cisternally, intra-vaginally, intra-peritoneally, intra-vascularly, locally (powder, ointment, or drops), via intra-tracheal intubation, intra-tracheal instillation, or as spray.

In an aspect, engineered (or transduced) γδ T cells can be expanded ex viγδ without stimulation by an antigen presenting cell or aminobisphosphonate. Antigen reactive engineered T cells of the present disclosure may be expanded ex viγδ and in vivo. In another aspect, an active population of engineered γδ T cells of the present disclosure may be expanded ex viγδ without antigen stimulation by an antigen presenting cell, an antigenic peptide, a non-peptide molecule, or a small molecule compound, such as an aminobisphosphonate but using certain antibodies, cytokines, mitogens, or fusion proteins, such as IL-17 Fc fusion, MICA Fc fusion, and CD70 Fc fusion. Examples of antibodies that can be used in the expansion of a γδ T-cell population may include anti-CD3, anti-CD27, anti-CD30, anti-CD70, anti-OX40, anti-NKG2D, or anti-CD2 antibodies, examples of cytokines may include IL-2, IL-15, IL-12, IL-21, IL-18, IL-9, IL-7, and/or IL-33, and examples of mitogens may include CD70 the ligand for human CD27, phytohaemagglutinin (PHA), concavalin A (ConA), pokeweed mitogen (PWM), protein peanut agglutinin (PNA), soybean agglutinin (SBA), lens culinaris agglutinin (LCA), pisum sativum agglutinin (PSA),h pomatia agglutinin (HPA), vicia graminea Lectin (VGA) or another suitable mitogen capable of stimulating T-cell proliferation. In another aspect, a population of engineered γδ T cells can be expanded in less than 60 days, less than 48 days, 36 days, less than 24 days, less than 12 days, or less than 6 days.

In another aspect, the present disclosure provides methods for the ex viγδ expansion of a population of engineered γδ T-cells for adoptive transfer therapy. Engineered γδ T cells of the disclosure may be expanded ex vivo. Engineered γδ T cells of the disclosure can be expanded in vitro without activation by APCs, or without co-culture with APCs, and aminophosphates.

In another aspect, a γδ T-cell population can be expanded in vitro in fewer than 36 days, fewer than 35 days, fewer than 34 days, fewer than 33 days, fewer than 32 days, fewer than 31 days, fewer than 30 days, fewer than 29 days, fewer than 28 days, fewer than 27 days, fewer than 26 days, fewer than 25days, fewer than 24 days, fewer than 23 days, fewer than 22 days, fewer than 21 days, fewer than 20 days, fewer than 19 days, fewer than 18 days, fewer than 17 days, fewer than 16 days, fewer than 15 days, fewer than 14 days, fewer than 13 days, fewer than 12 days, fewer than 11 days, fewer than 10 days, fewer than 9days, fewer than 8 days, fewer than 7 days, fewer than 6 days, fewer than 5 days, fewer than 4 days, or fewer than 3 days.

FIG. 2 shows expansion of the transduced or engineered γδ T cells may be carried out in the presence of cytokines, e.g., IL-2, IL-15, IL-18, and others, in small/mid-scale, e.g., flasks/G-Rex, or in large scale, e.g., 50 ml-100-liter bags, for 7-35 days, e.g., 14-28 days.

In some aspects, a γδ T-cell population can be re-stimulated one or more times during expansion. For example, an engineered (or transduced) γδ T-cell population may be expanded ex viγδ for a period of time and then restimulated by contacting the expanded γδ T cells with a feeder cell. For example, the feeder cell may be a monocyte, a PBMC, or a combination of monocytes and PBMC. In other aspects, the γδ T-cell population is not re-stimulated during expansion.

In some aspects, the feeder cell is autologous to the human subject. In an aspect, the feeder cell is allogenic to the human subject.

In some aspects, the feeder cell is depleted of αβ T cells.

In some aspects, the feeder cell is pulsed with an aminobisphosphonate, such as zoledronic acid, prior to addition to the γδ T-cell population.

In another aspect, the feeder cell may be a cell line, such as a tumor cell line or a lymphoblastoid cell line. In another aspect, the feeder cell may be a tumor cell, such as an autologous tumor cell. In an aspect, the tumor cell may be a K562 cell. In some aspects, the feeder cell is an engineered tumor cell comprising at least one recombinant protein, such as, for example, a cytokine. The cytokine can be, for example, CD86, 4-1BBL, IL-15, and any combination thereof. In some aspects, the IL-15 is membrane bound IL-15.

In some aspects the feeder cell is a combination of any feeder cells described herein. For example, the feeder cell may be a combination of two or more feeder cells selected from autologous monocytes, allogenic monocytes, autologous PBMC, allogenic PBMC, a tumor cell, an autologous tumor cell, an engineered tumor cell, a K562 cell, a tumor cell line, and a lymphoblastoid cell line. In some aspects, the feeder cell is a combination of PBMC and a lymphoblastoid cell line.

In some aspects, the feeder cell is irradiated, for example, γ-irradiated.

In some aspects, the expanded γδ T cells and the feeder cell is present in a ratio of from about 1:1 to about 50:1 (feeder cells: expanded γδ T cells). For example, the expanded γδ T cells and the feeder cell is present in a ratio of from about 2:1 to about 20:1 (feeder cells: expanded γδ T cells). In some aspects, the expanded γδ T cells and the feeder cell is present in a ratio of about 1:1, about 1:5:1, about 2:1, about 3:1, about 4:1, about 5:1, about 6:1, about 7:1, about 8:1, about 9:1, about 10:1, about 11:1, about 12:1, about 13:1, about 14:1, about 15:1, about 20:1, about 25:1, about 30:1, about 35:1, about 40:1, about 45:1or about 50:1 (feeder cells: expanded γδ T cells).

In some aspects, an expanded γδ T cell population of the present disclosure may be restimulated using certain antibodies, cytokines, mitogens, or fusion proteins, such as IL-17 Fc fusion, MICA Fc fusion, and CD70 Fc fusion. Examples of antibodies that can be used to restimulate an expanded γδ T-cell population may include anti-CD3, anti-CD27, anti-CD30, anti-CD70, anti-OX40, anti-NKG2D, or anti-CD2antibodies, examples of cytokines may include IL-2, IL-15, IL-12, IL-21, IL-18, IL-9, IL-7, and/or IL-33, and examples of mitogens may include CD70 the ligand for human CD27, phytohaemagglutinin (PHA), concavalin A (ConA), pokeweed mitogen (PWM), protein peanut agglutinin (PNA), soybean agglutinin (SBA), lens culinaris agglutinin (LCA), pisum sativum agglutinin (PSA),h pomatia agglutinin (HPA), vicia graminea Lectin (VGA) or another suitable mitogen capable of stimulating T-cell proliferation.

Restimulation of the expanded γδ T cells can be performed by contacting the expanded γδ T cells with any combination of the restimulation agents, such as feeder cells, antibodies, cytokines, mitogens, fusion proteins, etc., described herein.

In some aspects, the expanded γδ T cells are restimulated once during expansion. In other aspects, the expanded γδ T cells are restimulated more than once during expansion. For example, the expanded γδ T cells can be restimulated twice, three times, four times, five times, six times, seven times, eight times, nine times, or ten or more times during expansion. One of skill in the art can readily optimize the number of restimulations performed during expansion depending upon the conditions and length of the expansion.

In some aspects, the expanded γδ T cells are restimulated every day during expansion. In some aspects, the expanded γδ T cells are restimulated more than once a day during expansion. In other aspects, the expanded γδ T cells are restimulated once every two days, once every three days, once every four days, once every five days, once every six days, once every seven days, once every eight days, once every nine days, once every ten days, once every eleven days, once every twelve days, once every thirteen days, once every fourteen days, etc. In other aspects, the expanded γδ T cells are restimulated once a week, twice a week, three times a week, four times a week, five times a week, six times a week, etc. In other aspects, the expanded γδ T cells are restimulated once every two weeks, once every three weeks, once every four weeks, etc. One of skill in the art can readily optimize the length of time between restimulations performed during expansion depending upon the conditions and length of the expansion.

It will be understood that when multiple restimulations are performed during the expansion, each restimulation may be identical or different. For example, each restimulation may be performed using any combination of restimulation agents described herein in any amount. The specific restimulation agents used and amounts thereof may be the same or different for each restimulation.

The expanded transduced T cell products may then be cryopreserved as “off-the-shelf” T-cell products for infusion into patients.

### Methods of Treatment

Compositions containing engineered γδ T cells described herein may be administered for prophylactic and/or therapeutic treatments. In therapeutic applications, pharmaceutical compositions can be administered to a subject already suffering from a disease or condition in an amount sufficient to cure or at least partially arrest the symptoms of the disease or condition. An engineered γδ T-cell can also be administered to lessen a likelihood of developing, contracting, or worsening a condition. Effective amounts of a population of engineered γδ T-cells for therapeutic use can vary based on the severity and course of the disease or condition, previous therapy, the subject's health status, weight, and/or response to the drugs, and/or the judgment of the treating physician.

Engineered γδ T cells of the present disclosure can be used to treat a subject in need of treatment for a condition, for example, a cancer, an infectious disease, and/or an immune disease described herein.

A method of treating a condition (e.g., ailment) in a subject with γδ T cells may include administering to the subject a therapeutically-effective amount of engineered γδ T cells. γδ T cells of the present disclosure may be administered at various regimens (e.g., timing, concentration, dosage, spacing between treatment, and/or formulation). A subject can also be preconditioned with, for example, chemotherapy, radiation, or a combination of both, prior to receiving engineered γδ T cells of the present disclosure. A population of engineered γδ T cells may also be frozen or cryopreserved prior to being administered to a subject. A population of engineered γδ T cells can include two or more cells that express identical, different, or a combination of identical and different tumor recognition moieties. For instance, a population of engineered γδ T-cells can include several distinct engineered γδ T cells that are designed to recognize different antigens, or different epitopes of the same antigen.

γδ T cells of the present disclosure may be used to treat various conditions. In an aspect, engineered γδ T cells of the present disclosure may be used to treat a cancer, including solid tumors and hematologic malignancies. Non-limiting examples of cancers include: acute lymphoblastic leukemia, acute myeloid leukemia, adrenocortical carcinoma, AIDS-related cancers, AIDS-related lymphoma, anal cancer, appendix cancer, astrocytomas, neuroblastoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, μleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, Merkel cell skin carcinoma, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom's macroglobulinemia, and Wilms tumor.

In an aspect, engineered γδ T cells of the present disclosure may be used to treat an infectious disease, such as viral or bacterial infections, for example dengue fever, Ebola, Marburg virus, tuberculosis (TB), meningitis or syphilis, preferable the method is used on antibiotic-resistant strains of infectious organisms, autoimmune diseases, parasitic infections, such as malaria and other diseases such as MS and Morbus Parkinson, as long as the immune answer is a MHC class I answer.

In yet another aspect, engineered γδ T cells of the present disclosure may be used to treat an immune disease, such as an autoimmune disease. Examples for autoimmune diseases (including diseases not officially declared to be autoimmune diseases) are Arthritis, Chronic obstructive pulmonary disease, Ankylosing Spondylitis, Crohn's Disease (one of two types of idiopathic inflammatory bowel disease “IBD”), Dermatomyositis, Diabetes mellitus type 1, Endometriosis, Goodpasture's syndrome, Graves' disease, Guillain-Barre syndrome (GBS), Hashimoto's disease, Hidradenitis suppurativa, Kawasaki disease, IgA nephropathy, Idiopathic thrombocytopeniarpura, Interstitial cystitis, Lupus erythematosus, Mixed Connective Tissue Disease, Morphea, Myasthenia gravis, Narcolepsy, Neuromyotonia, Pemphigus vulgaris, Pernicious anaemia, Psoriasis, Psoriatic Arthritis, Polymyositis, Primary biliary cirrhosis, Relapsing polychondritis, Rheumatoid arthritis, Schizophrenia, Scleroderma, Sjogren's syndrome, Stiff person syndrome, Temporal arteritis (also known as “giant cell arteritis”), Ulcerative Colitis (one of two types of idiopathic inflammatory bowel disease “IBD”), Vasculitis, Vitiligo and Wegener's granulomatosis.

Treatment with γδ T cells of the present disclosure may be provided to the subject before, during, and after the clinical onset of the condition. Treatment may be provided to the subject after 1 day, 1 week, 6 months, 12 months, or 2 years after clinical onset of the disease. Treatment may be provided to the subject for more than 1 day, 1 week, 1 month, 6 months, 12 months, 2 years, 3 years, 4 years, 5 years, 6 years, 7 years, 8 years, 9 years, 10 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can include administering to a subject a pharmaceutical composition comprising engineered γδ T cells of the present disclosure.

In another aspect, administration of engineered γδ T cells of the present disclosure to a subject may modulate the activity of endogenous lymphocytes in a subject's body. In another aspect, administration of engineered γδ T cells to a subject may provide an antigen to an endogenous T-cell and may boost an immune response. In another aspect, the memory T cell may be a CD4+ T-cell. In another aspect, the memory T cell may be a CD8+ T-cell. In another aspect, administration of engineered γδ T cells of the present disclosure to a subject may activate the cytotoxicity of another immune cell. In another aspect, the other immune cell may be a CD8+ T-cell. In another aspect, the other immune cell may be a Natural Killer T-cell. In another aspect, administration of engineered γδ T-cells of the present disclosure to a subject may suppress a regulatory T-cell. In another aspect, the regulatory T-cell may be a FOX3+ Treg cell. In another aspect, the regulatory T-cell may be a FOX3-Treg cell. Non-limiting examples of cells whose activity can be modulated by engineered γδ T cells of the disclosure may include: hematopoietic stem cells; B cells; CD4; CD8; red blood cells; white blood cells; dendritic cells, including dendritic antigen presenting cells; leukocytes; macrophages; memory B cells; memory T-cells; monocytes; natural killer cells; neutrophil granulocytes; T-helper cells; and T-killer cells.

During most bone marrow transplants, a combination of cyclophosphamide with total body irradiation may be conventionally employed to prevent rejection of the hematopoietic stem cells (HSC) in the transplant by the subject's immune system. In an aspect, incubation of donor bone marrow with interleukin-2 (IL-2) ex viγδ may be performed to enhance the generation of killer lymphocytes in the donor marrow. Interleukin-2 (IL-2) is a cytokine that may be necessary for the growth, proliferation, and differentiation of wild-type lymphocytes. Current studies of the adoptive transfer of γδ T-cells into humans may require the co-administration of γδ T-cells and interleukin-2. However, both low-and high-dosages of IL-2 can have highly toxic side effects. IL-2 toxicity can manifest in multiple organs/systems, most significantly the heart, lungs, kidneys, and central nervous system. In another aspect, the disclosure provides a method for administrating engineered γδ T cells to a subject without the co-administration of a native cytokine or modified versions thereof, such as IL-2, IL-15, IL-12, IL-21. In another aspect, engineered γδ T cells can be administered to a subject without co-administration with IL-2. In another aspect, engineered γδ T cells may be administered to a subject during a procedure, such as a bone marrow transplant without the co-administration of IL-2.

### Methods of Administration

One or multiple engineered γδ T cell populations may be administered to a subject in any order or simultaneously. If simultaneously, the multiple engineered γδ T cell can be provided in a single, unified form, such as an intravenous injection, or in multiple forms, for example, as multiple intravenous infusions, s.c. injections or pills. Engineered γδ T-cells can be packed together or separately, in a single package or in a plurality of packages. One or all of the engineered γδ T cells can be given in multiple doses. If not simultaneous, the timing between the multiple doses may vary to as much as about a week, a month, two months, three months, four months, five months, six months, or about a year. In another aspect, engineered γδ T cells can expand within a subject's body, in vivo, after administration to a subject. Engineered γδ T cells can be frozen to provide cells for multiple treatments with the same cell preparation. Engineered γδ T cells of the present disclosure, and pharmaceutical compositions comprising the same, can be packaged as a kit. A kit may include instructions (e.g., written instructions) on the use of engineered γδ T cells and compositions comprising the same.

In another aspect, a method of treating a cancer, infectious disease, or immune disease comprises administering to a subject a therapeutically-effective amount of engineered γδ T cells, in which the administration treats the cancer, infectious disease, or immune disease. In another embodiments, the therapeutically-effective amount of engineered γδ T cells may be administered for at least about 10seconds, 30 seconds, 1 minute, 10 minutes, 30 minutes, 1 hour, 2 hours, 3 hours, 4 hours, 5 hours, 6 hours, 12 hours, 24 hours, 2 days, 3 days, 4 days, 5 days, 6 days, 1 week, 2 weeks, 3 weeks, 1 month, 2 months, 3months, 4 months, 5 months, 6 months, or 1 year. In another aspect, the therapeutically-effective amount of the engineered γδ T cells may be administered for at least one week. In another aspect, the therapeutically-effective amount of engineered γδ T cells may be administered for at least two weeks.

Engineered γδ T-cells described herein can be administered before, during, or after the occurrence of a disease or condition, and the timing of administering a pharmaceutical composition containing an engineered γδ T-cell can vary. For example, engineered γδ T cells can be used as a prophylactic and can be administered continuously to subjects with a propensity to conditions or diseases in order to lessen a likelihood of the occurrence of the disease or condition. Engineered γδ T-cells can be administered to a subject during or as soon as possible after the onset of the symptoms. The administration of engineered γδ T cells can be initiated immediately within the onset of symptoms, within the first 3 hours of the onset of the symptoms, within the first 6 hours of the onset of the symptoms, within the first 24 hours of the onset of the symptoms, within 48 hours of the onset of the symptoms, or within any period of time from the onset of symptoms. The initial administration can be via any route practical, such as by any route described herein using any formulation described herein. In another aspect, the administration of engineered γδ T cells of the present disclosure may be an intravenous administration. One or multiple dosages of engineered γδ T cells can be administered as soon as is practicable after the onset of a cancer, an infectious disease, an immune disease, sepsis, or with a bone marrow transplant, and for a length of time necessary for the treatment of the immune disease, such as, for example, from about 24 hours to about 48 hours, from about 48 hours to about 1 week, from about 1 week to about 2 weeks, from about 2 weeks to about 1 month, from about 1 month to about 3 months. For the treatment of cancer, one or multiple dosages of engineered γδ T cells can be administered years after onset of the cancer and before or after other treatments. In another aspect, engineered γδ T cells can be administered for at least about 10minutes, 30 minutes, 1 hour, 2 hours, 3 hours, 4 hours, 5 hours, 6 hours, 12 hours, 24 hours, at least 48 hours, at least 72 hours, at least 96 hours, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years at least 3 years, at least 4 years, or at least 5 years. The length of treatment can vary for each subject.

### Preservation

In an aspect, γδ T cells may be formulated in freezing media and μlaced in cryogenic storage units such as liquid nitrogen freezers (−196° C.) or ultra-low temperature freezers (−65° C., −80° C., −120° C., or −150° C.) for long-term storage of at least about 1 month, 2 months, 3 months, 4 months, 5 months, 6 months, 1 year, 2 years, 3 years, or at least 5 years. The freeze media can contain dimethyl sulfoxide (DMSO), and/or sodium chloride (NaCl), and/or dextrose, and/or dextran sulfate and/or hydroxyethyl starch (HES) with physiological pH buffering agents to maintain pH between about 6.0 to about 6.5, about 6.5 to about 7.0, about 7.0 to about 7.5, about 7.5 to about 8.0 or about 6.5 to about 7.5. The cryopreserved γδ T cells can be thawed and further processed by stimulation with antibodies, proteins, peptides, and/or cytokines as described herein. The cryopreserved γδ T-cells can be thawed and genetically modified with viral vectors (including retroviral, adeno-associated virus (AAV), and lentiviral vectors) or non-viral means (including RNA, DNA, e.g., transposons, and proteins) as described herein. The modified γδ T cells can be further cryopreserved to generate cell banks in quantities of at least about 1, 5, 10, 100, 150, 200, 500 vials at about at least 10¹, 10², 10³, 10⁴, 10⁵, 10⁶, 10⁷, 10⁸, 10⁹, or at least about 10¹⁰ cells per mL in freeze media. The cryopreserved cell banks may retain their functionality and can be thawed and further stimulated and expanded. In another aspect, thawed cells can be stimulated and expanded in suitable closed vessels, such as cell culture bags and/or bioreactors, to generate quantities of cells as allogeneic cell product. Cryopreserved γδ T cells can maintain their biological functions for at least about 6 months, 7 months, 8months, 9 months, 10 months, 11 months, 12 months, 13 months, 15 months, 18 months, 20 months, 24months, 30 months, 36 months, 40 months, 50 months, or at least about 60 months under cryogenic storage condition. In another aspect, no preservatives may be used in the formulation. Cryopreserved γδ T-cells can be thawed and infused into multiple patients as allogeneic off-the-shelf cell product.

In an aspect, engineered γδ T-cell described herein may be present in a composition in an amount of at least 1×10³ cells/ml, at least 2×10³ cells/ml, at least 3×10³ cells/ml, at least 4×10³ cells/ml, at least 5×10³ cells/ml, at least 6×10³ cells/ml, at least 7×10³ cells/ml, at least 8×10³ cells/ml, at least 9×10³ cells/ml, at least 1×10⁴ cells/ml, at least 2×10⁴ cells/ml, at least 3×10⁴ cells/ml, at least 4×10⁴ cells/ml, at least 5×10⁴ cells/ml, at least 6×10⁴ cells/ml, at least 7×10⁴ cells/ml, at least 8×10⁴ cells/ml, at least 9×10⁴ cells/ml, at least 1×10⁵ cells/ml, at least 2×10⁵ cells/ml, at least 3×10⁵ cells/ml, at least 4×10⁵ cells/ml, at least 5×10⁵ cells/ml, at least 6×10⁵ cells/ml, at least 7×10⁵ cells/ml, at least 8×10⁵ cells/ml, at least 9×10⁵ cells/ml, at least 1×10⁶ cells/ml, at least 2×10⁶ cells/ml, at least 3×10⁶ cells/ml, at least 4×10⁶ cells/ml, at least 5×10⁶ cells/ml, at least 6×10⁶ cells/ml, at least 7×10⁶ cells/ml, at least 8×10⁶ cells/ml, at least 9×10⁶ cells/ml, at least 1×10⁷ cells/ml, at least 2×10⁷ cells/ml, at least 3×10⁷ cells/ml, at least 4×10⁷ cells/ml, at least 5×10⁷ cells/ml, at least 6×10⁷ cells/ml, at least 7×10⁷ cells/ml, at least 8×10⁷ cells/ml, at least 9×10⁷ cells/ml, at least 1×10⁸ cells/ml, at least 2×10⁸ cells/ml, at least 3×10⁸ cells/ml, at least 4×10⁸ cells/ml, at least 5×10⁸ cells/ml, at least 6×10⁸ cells/ml, at least 7×10⁸ cells/ml, at least 8×10⁸ cells/ml, at least 9×10⁸ cells/ml, at least 1×10⁹ cells/ml, or more, from about 1×10³ cells/ml to about at least 1×10⁸ cells/ml, from about 1×10⁵ cells/ml to about at least 1×10⁸ cells/ml, or from about 1×10⁶ cells/ml to about at least 1×10⁸ cells/ml.

To develop viable allogeneic T cell products, e.g., that can be engineered to express tumor antigen specific TCR, e.g., chimeric CD8α-CD4tm/intracellular protein, embodiments of the present disclosure may include methods that can maximize the yield of [symbol] [symbol] T cells while minimizing the presence of residual [symbol] [symbol] T cells in the final allogeneic products. For example, embodiments of the present disclosure may include methods of expanding and activating γδ T cells by depleting [symbol] [symbol] T cells and supplementing the growth culture with molecules, such as Amphotericin B, N-acetyl cysteine (NAC) (or high dose glutamine/glutamax), IL-2, and/or IL-15.

In an aspect, methods described herein may be used to produce autologous or allogenic products according to an aspect of the disclosure.

The present invention may be better understood by reference to the following examples, which are not intended to limit the scope of the claims.

### EXAMPLES

### Example 1

Re-stimulation of γδ T cells during expansion with autologous cells leads to enhanced and prolonged expansion.

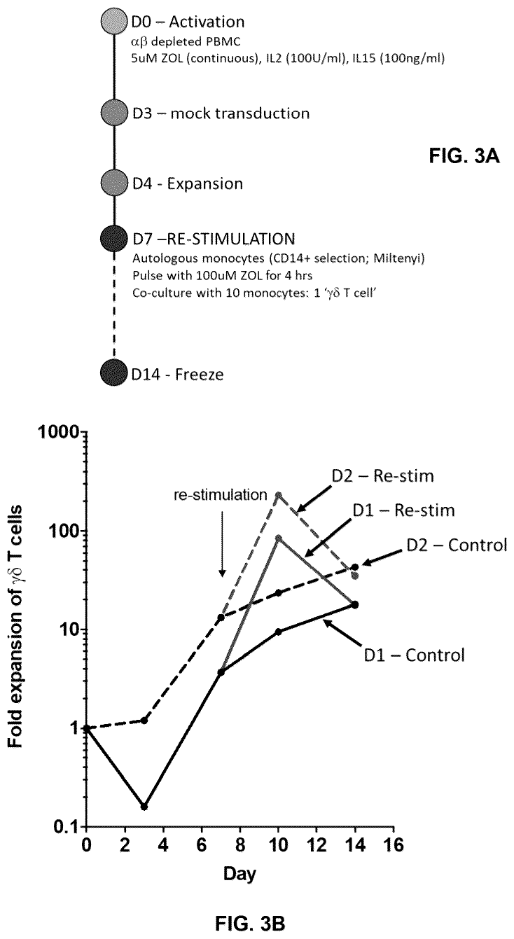

FIGS. 3A and 3B show the effect of re-stimulation with autologous monocytes on the expansion of γδ T cells. FIG. 3A shows the expansion process used to generate the data presented in FIG. 3B. Briefly, on Day 0, the αβ-TCR expressing T cell (including CD4+ and CD8+ T cells)-depleted peripheral blood mononuclear cells (PBMC) (“yõ T cells”) were activated in the presence of zoledronate (ZOL) (5 μM), IL-2 (100 U/ml), and IL-15 (100 ng/ml). On Day 3, the activated γδ T cells were mock transduced. On Day 4, the mock-transduced cells are expanded. On Day 7, the expanded cells were re-stimulated with autologous monocytes (obtained by CD14+selection from PBMC (Miltenyi) and pulsed with ZOL (100 μM) for 4 hours) at a ratio of 10 (monocytes):1 (γδ T cells). The expanded cells were frozen on Day 14.

FIG. 3B shows re-stimulation with autologous monocytes increases fold-expansion of γδ T cells obtained from two donors (D1 and D2) as compared with that without re-stimulation. The fold expansion of the re-stimulated cells decreased after 10 days. By 14 days, the fold expansion of the re-stimulated cells decreased to fold expansion similar to that without re-stimulation.

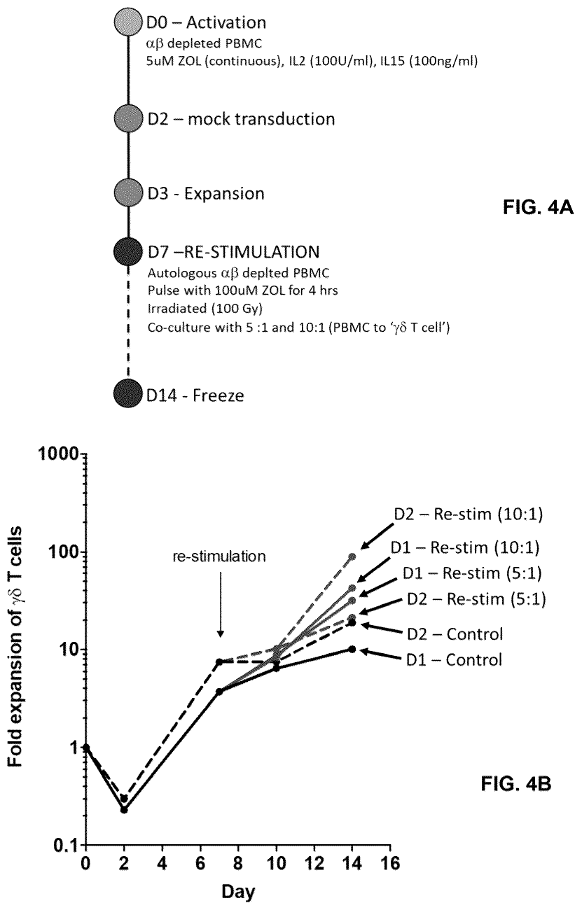

FIGS. 4A and 4B show the effect of re-stimulation with irradiated autologous αβ depleted PBMC on the expansion of γδ T cells. FIG. 4A shows the expansion process used to generate the data presented in FIG. 4B. Briefly, on Day 0, the αβ-TCR expressing T cells (including CD4+ and CD8+ T cells) depleted peripheral blood mononuclear cells (PBMC) (“γδ T cells”) were activated in the presence of zoledronate (ZOL) (5 μM), IL-2 (100 U/ml), and IL-15 (100 ng/ml). On Day 2, the activated γδ T cells were mock transduced. On Day 3, the mock-transduced cells are expanded. On Day 7, the expanded cells were re-stimulated with irradiated (100 Gy) autologous αβ-TCR expressing T cells depleted PBMC (pulsed with ZOL (100μ) for 4 hours) at a ratio of 5:1 or 10:1 (αβ depleted PBMC: γδ T cells).

FIG. 4B shows re-stimulation with αβ depleted PBMC at 5:1 and 10:1 ratios increases fold-expansion of γδ T cells obtained from two donors (D1 and D2) as compared with that without re-stimulation.

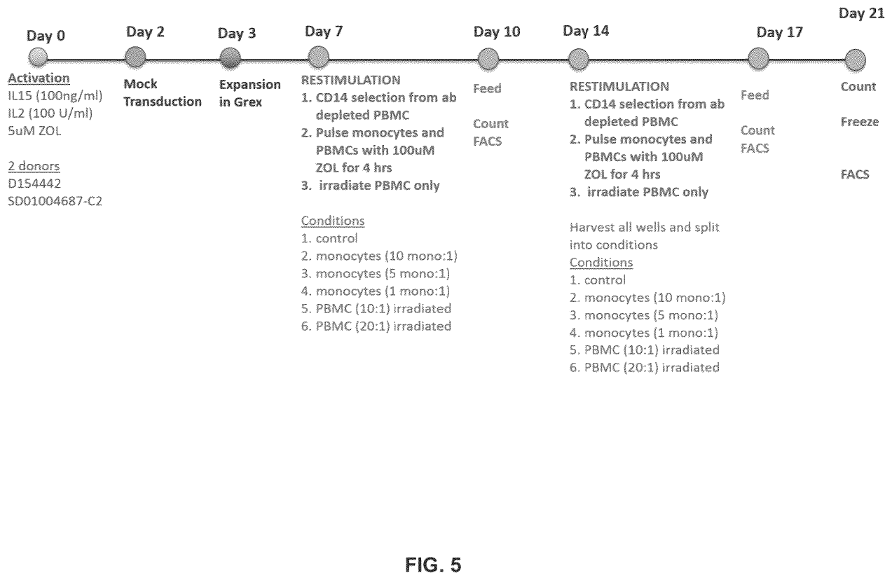

FIGS. 5-11 show the effect of multiple re-stimulations with autologous monocytes or irradiated autologous αβ depleted PBMC on the expansion of γδ T cells.

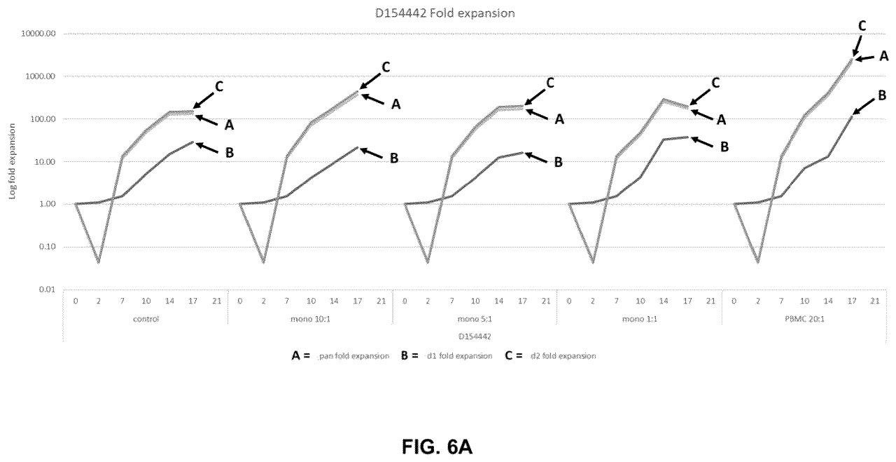

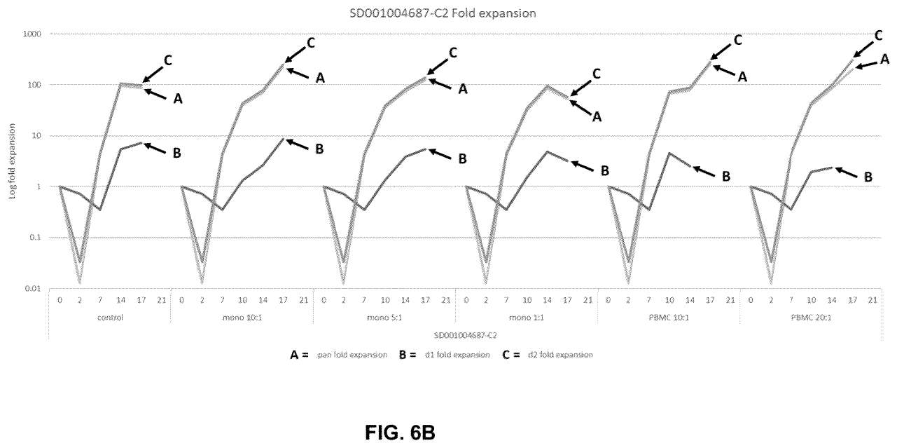

FIG. 5 shows the expansion process used to generate the data presented in FIGS. 6-11. Briefly, on Day 0, the αβ-TCR expressing T cells (including CD4+ and CD8+ T cells) depleted peripheral blood mononuclear cells (PBMC) (“γδ T cells”) were activated in the presence of zoledronate (ZOL) (5 μM), IL-2 (100 U/ml), and IL-15 (100 ng/ml). On Day 2, the activated γδ T cells were mock transduced. On Day 3, the mock-transduced cells are expanded. On Day 7 and on Day 14, the expanded cells were re-stimulated with either 1) autologous monocytes (obtained by CD14+ selection from PBMC (Miltenyi) and pulsed with ZOL (100 μM) for 4 hours) at a ratio of 1:1, 5:1 or 10:1 (monocytes: γδ T cells) or 2) irradiated (100 Gy) autologous αβ-TCR expressing T cells depleted PBMC (pulsed with ZOL (100 μM) for 4 hours) at a ratio of 10:1 or 20:1 (αβ depleted PBMC: γδ T cells).

FIGS. 6A and 6B show the effect of multiple re-stimulations with autologous monocytes or irradiated autologous αβ depleted PBMC on the expansion of γδ T cells from two donors. FIG. 6A shows data from donor 1. In control samples and at lower ratios of monocytes: γδ T cells, expansion μlateaued by approximately Day 14. However, restimulation of γδ T cells with monocytes at a 10:1 ratio (monocyte: γδ T cells) or with irradiated αβ depleted PBMC at a 20:1 ratio (αβ depleted PBMC: γδ T cells) on Days 7 and 14prevented this μlateau, significantly enhancing expansion for at least 17 days. For example, δ2 cells reached a 2498 fold expansion on Day 17 when restimulated with irradiated αβ depleted PBMC at a 20:1 ratio (αβ depleted PBMC: γδ T cells) on Days 7 and 14 without reaching μlateau.

FIG. 6B shows the effect of multiple re-stimulations with autologous monocytes or irradiated autologous αβ depleted PBMC on the expansion of γδ T cells from a second donor. Similar to the data shown in FIG. 5B, expansion μlateaued by approximately Day 14 in control samples and at lower ratios of monocytes: γδ T cells. However, restimulation of γδ T cells with monocytes at a 5:1 or 10:1 ratio (monocyte: γδ T cells) or with irradiated αβ depleted PBMC at a 10:1 or 20:1 ratio (αβ depleted PBMC: γδ T cells) on Days 7 and 14 prevented this μlateau, significantly enhancing expansion for at least 17 days. For example, δ2 cells reached a 305 fold expansion on Day 17 when restimulated with irradiated αβ depleted PBMC at a 20:1ratio (αβ depleted PBMC: γδ T cells) on Days 7 and 14 without reaching μlateau.

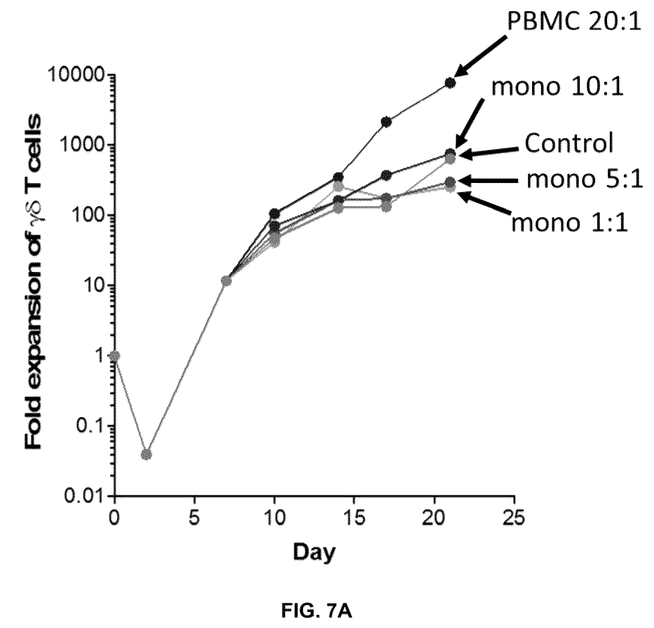

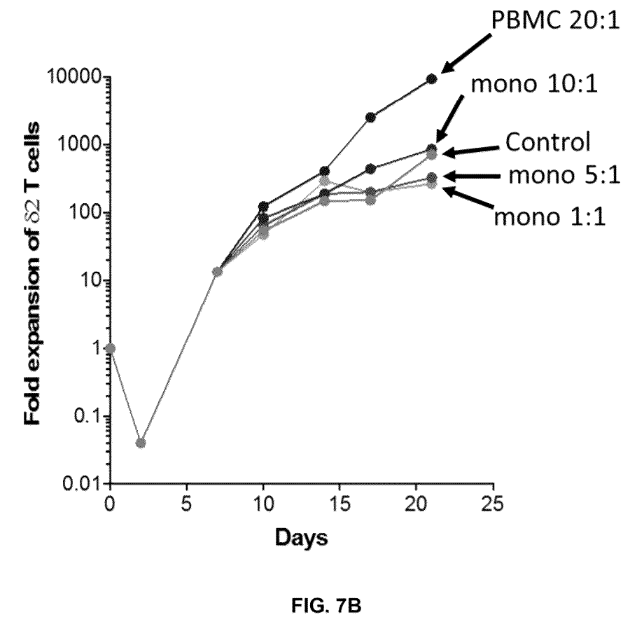

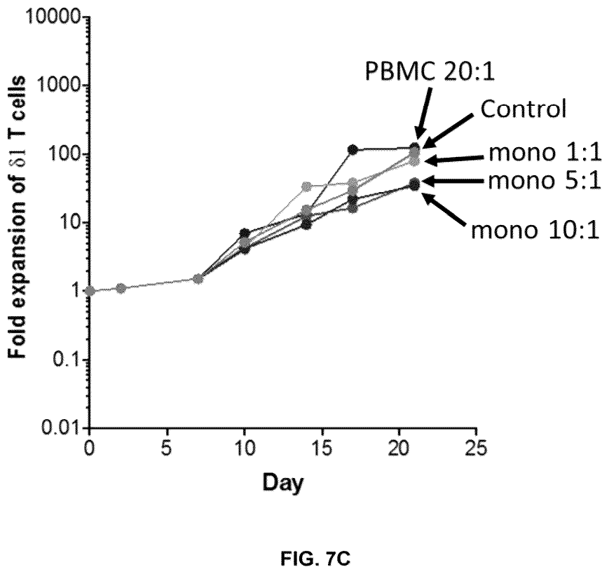

FIGS. 7A-C and 8A-C show the effect of multiple re-stimulations with autologous monocytes or irradiated autologous αβ depleted PBMC on the expansion of γδ T cells from two donors. These data are also summarized below in Table 1.

TABLE 1
Fold change in expansion compared
to control conditions at Day 21.

| Donor | Feeder Cell | pan γδ T cells | δ2 T cells |
|---|---|---|---|
| D1 | monocytes 10:1 | 1.2 | 1.2 |
| | monocytes 5:1 | 0.5 | 0.5 |
| | monocytes 1:1 | 0.4 | 0.4 |
| | PBMC 20:1 | 12.2 | 13.2 |
| | PBMC 10:1 | — | — |
| D2 | monocytes 10:1 | 5.5 | 5.6 |
| | monocytes 5:1 | 2.6 | 2.6 |
| | monocytes 1:1 | 1.7 | 1.7 |
| | PBMC 20:1 | 18.8 | 19.2 |
| | PBMC 10:1 | 15.6 | 67.8 |

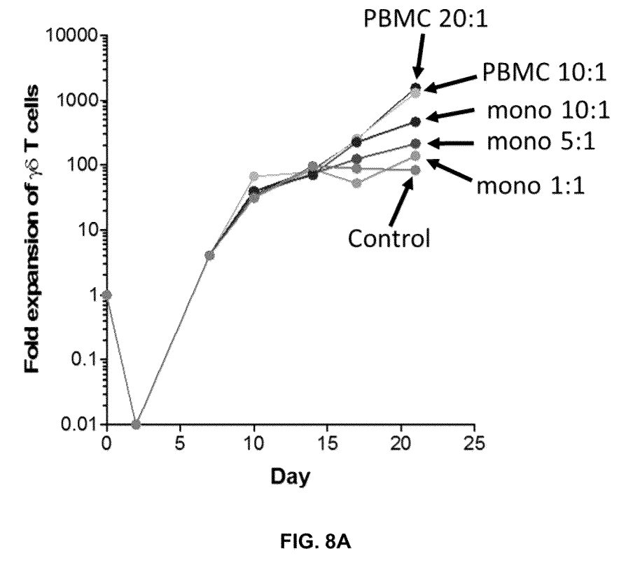

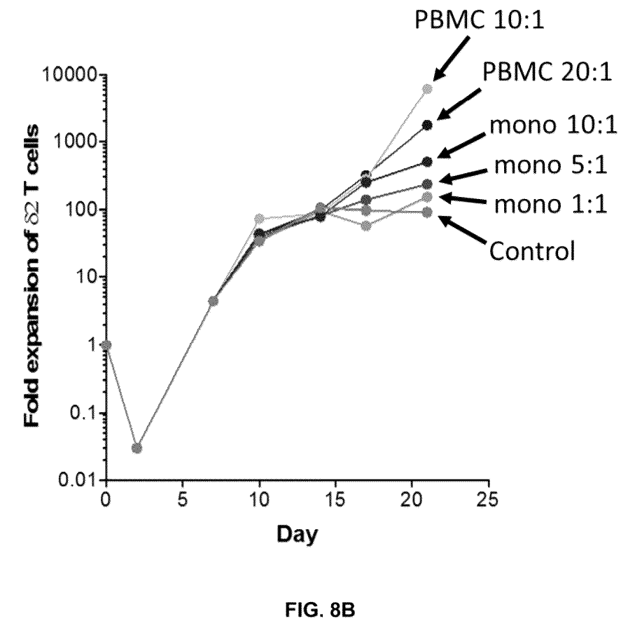

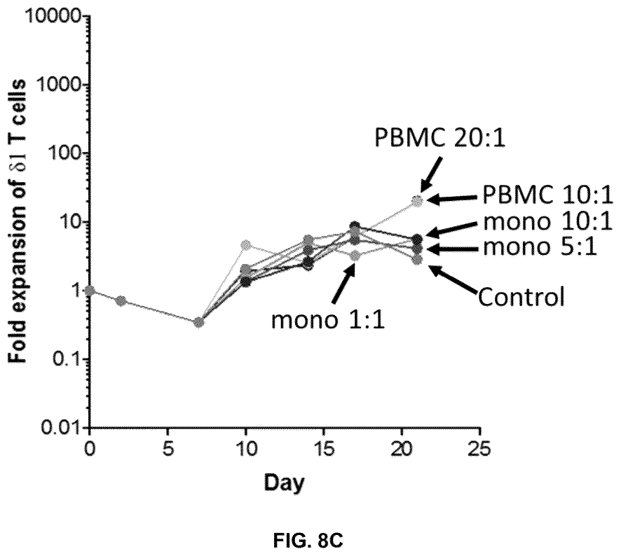

As seen in Table 1 and FIGS. 7A-C, the fold-expansion was lower in donor 1 compared to donor 2 (see FIGS. 8A-C). This result can be attributed to a sudden increase in expansion of control samples seen on Day 21. Despite this, it is clear that re-stimulation with irradiated autologous αβ depleted PMBCs results in higher fold-expansion of total γδ T cells compared to re-stimulation with autologous monocytes in both donors. This effect appears to be primarily due to an increase in δ2 T cells.

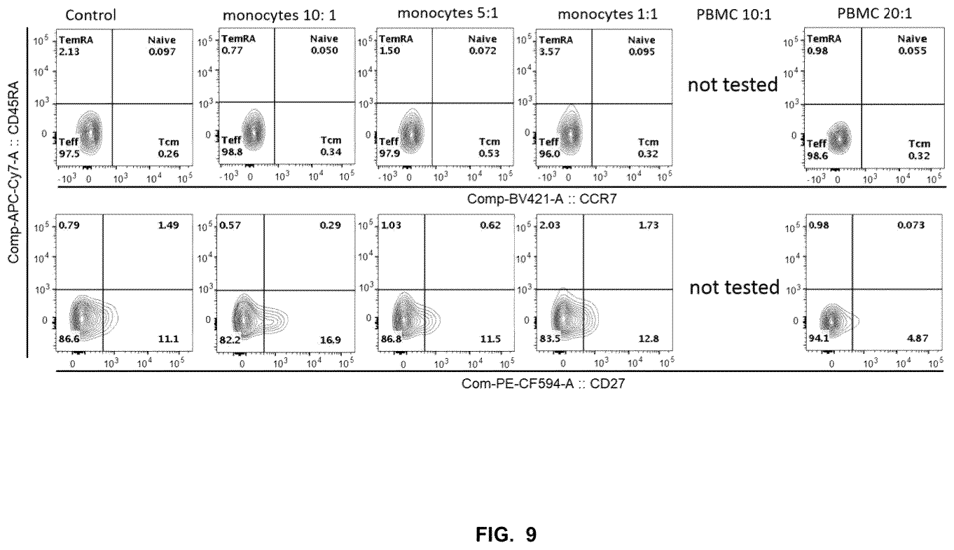

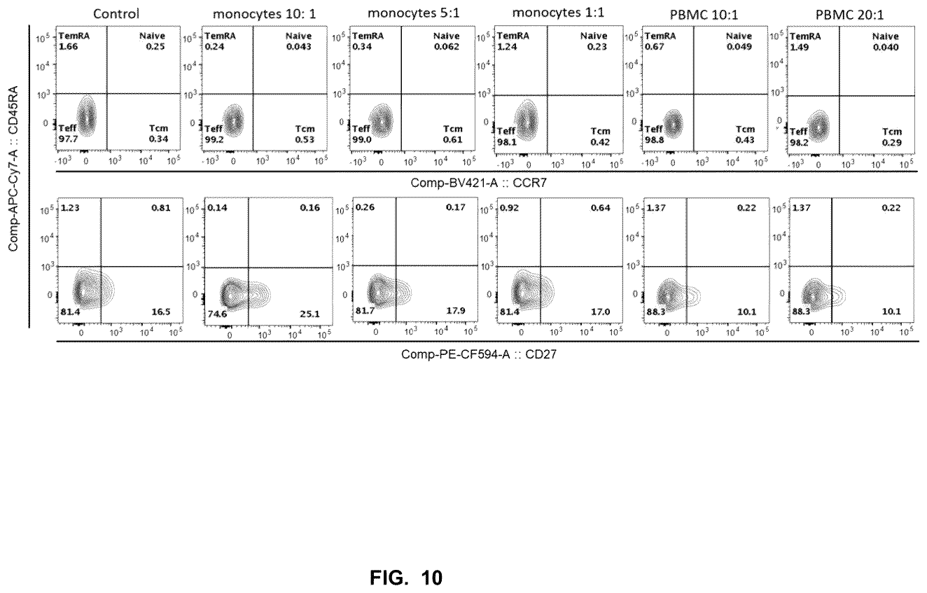

FIGS. 9 and 10 shows that multiple re-stimulations with autologous monocytes or irradiated autologous αβ depleted PBMC does not significantly alter the memory phenotype of expanded γδ T cells. A slight increase in CD27 expression was detected in expanded γδ T cells re-stimulated with 10:1 monocytes in both donors.

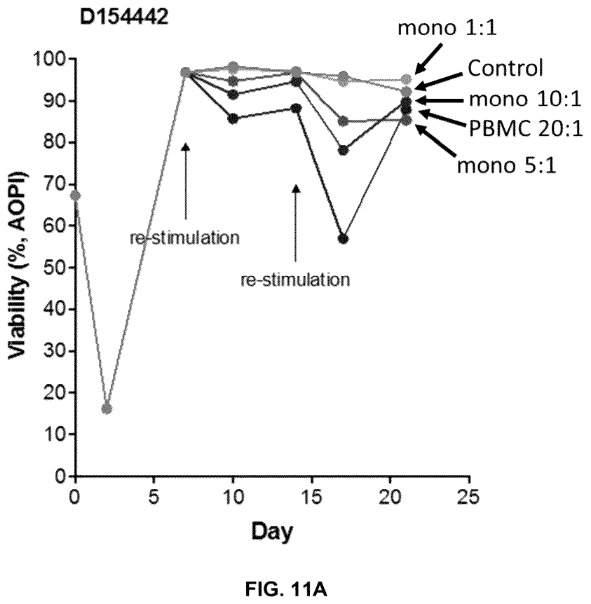

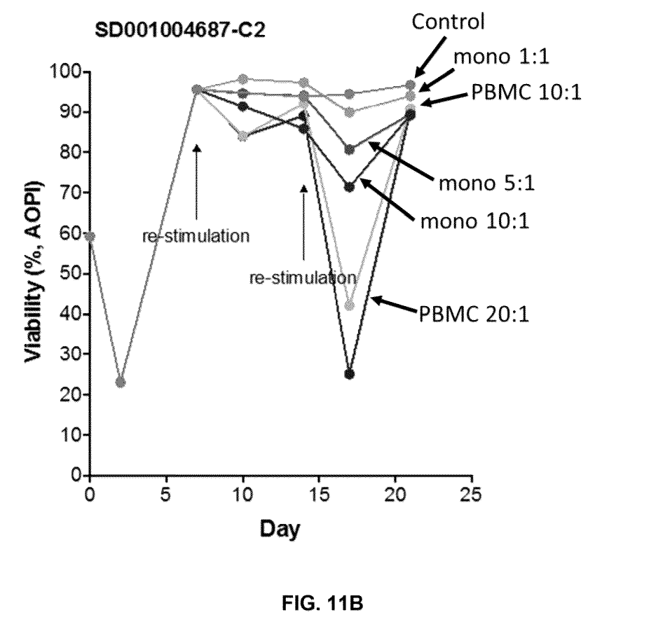

FIGS. 11A and 11B show the effect of multiple re-stimulations with autologous monocytes or irradiated autologous αβ depleted PBMC on viability of expanded γδ T cells. A decrease in viability of expanded γδ T cells was seen in re-stimulation conditions. The effect was most pronounced in γδ T cells re-stimulated with irradiated autologous αβ depleted PBMC (20 PBMC:1 γδ T cell). Viability tends to decrease following re-stimulation and rebound within a week.

### Example 2

Stimulation of γδ T cells with tumor-derived cells enhances and prolongs expansion.

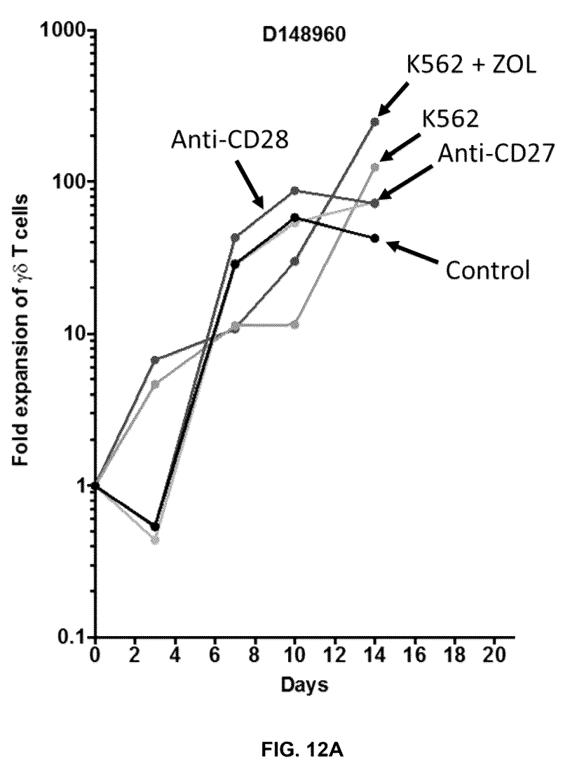

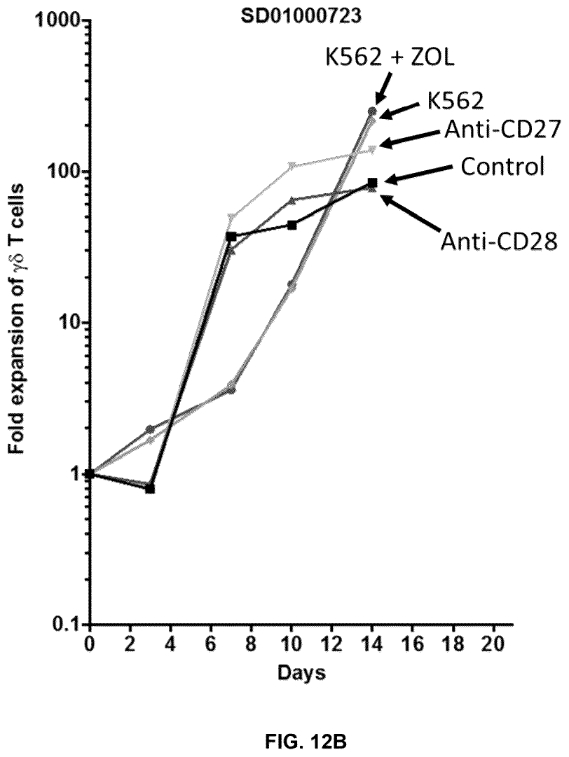

FIGS. 12A and 12B show the effect of co-culture of engineered tumor-derived cells on γδ T cells. Briefly, on Day 0, the αβ-TCR expressing T cells (including CD4+ and CD8+ T cells) depleted peripheral blood mononuclear cells (PBMC) (“γδ T cells”) were activated in the presence of zoledronate (ZOL) (5 μM), IL-2(100 U/ml), and IL-15 (100 ng/ml). Irradiated tumor-derived cells (K562) were added in a 2:1 ratio (tumor-derived cell:γδ T cells) to some samples. Other samples were cultured on anti-CD28 or anti-CD27 mAb-coated plates. On Day 3, the activated γδ T cells were mock transduced. On Day 4, the mock-transduced cells were expanded. Expanded cells were frozen on Day 21.

FIGS. 12A and 12B shows γδ T cells obtained from two donors (D1 (FIG. 12A) and D2 (FIG. 12B)) stimulated with irradiated tumor-derived cells+/−ZOL has higher fold expansion than that stimulated with anti-CD28 antibody+ZOL, anti-CD27 antibody+ZOL, and ZOL alone (control).

### Example 3

Stimulation of γδ T cells with tumor-derived cells with re-stimulation enhances and prolongs expansion of γδ T cells.

Table 2 summarizes the conditions tested in this experiment. Briefly, γδ T cells obtained from two donors were activated on Day 0 in the presence of IL-2 (100 U/ml), and IL-15 (100 ng/ml)+/−zoledronate (ZOL) (5 μM)+/−tumor-derived cells (2 tumor-derived cells:1 T cell)+/−re-stimulation as follows: a) in the absence of tumor-derived cells (control); b) with wild-type irradiated tumor-derived cells (K562 WT); c) with irradiated modified tumor-derived cells (K562 variant 2) in the absence of ZOL; c-Restim) with irradiated modified tumor-derived cells (K562 variant 2) in the absence of ZOL with re-stimulation on Days 7 and 14; d) with irradiated modified tumor-derived cells (K562 variant 2); and e) with irradiated modified tumor-derived cells (K562 variant 1). Cells were mock transduced on Day 2 and expanded on Day 3. Cells were fed on Days 7, 10, 14 and 17 and optionally re-stimulated on Days 7 and 14. Cells were frozen on Day 21.

TABLE 2

| | | Feeder Cell | Zoledronate | Re-Stim | Re-Stim |
|---|---|---|---|---|---|
| Donor | Sample # | (D0) | (5uM; D0) | (D7) | (D14) |
| D1 | 1a | N/A | + | − | − |
| | 1b | K562 WT | + | − | − |
| | 1c | K562 variant 2 | − | − | − |
| | 1c_Restim | K562 variant 2 | − | K562 variant 2 | K562 variant 2 |
| | 1d | K562 variant 2 | + | − | − |
| | 1e | K562 variant 1 | + | − | − |
| D2 | 2a | N/A | + | − | − |
| | 2b | K562 WT | + | − | − |
| | 2c | K562 variant 2 | − | − | − |
| | 2c_Restim | K562 variant 2 | − | K562 variant 2 | K562 variant 2 |
| | 2d | K562 variant 2 | + | − | − |
| | 2e | K562 variant 1 | + | − | − |

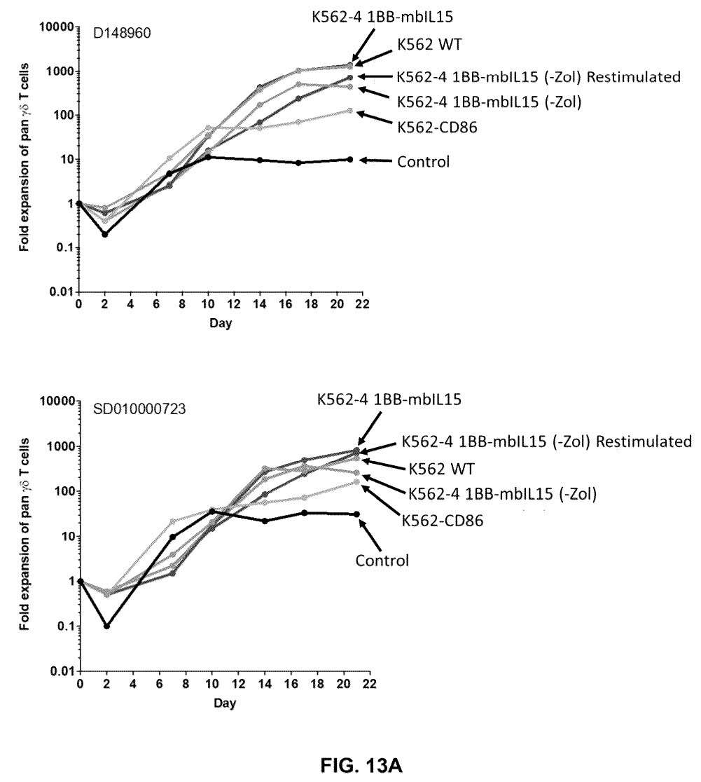

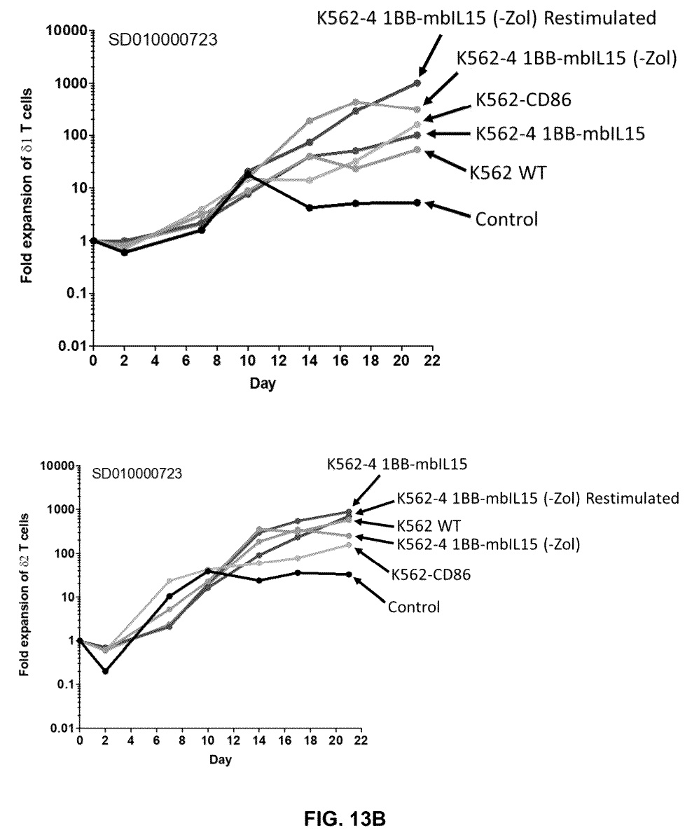

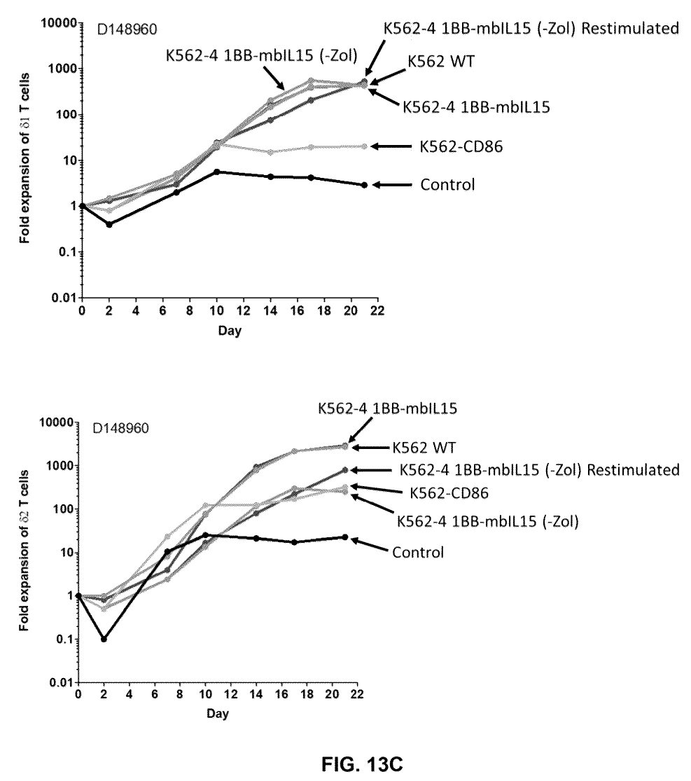

FIGS. 13A-C show results from co-culture of various tumor-derived cells during activation of γδ T cells. FIG. 13A shows fold expansion of γδ T cells obtained from two donors (D1 (left panel) and D2 (right panel)) activated on Day 0 in the presence of zoledronate (ZOL) (5 μM), IL-2 (100 U/ml), and IL-15 (100 ng/ml): 1) in the absence of tumor-derived cells (control); 2) with wild-type irradiated tumor-derived cells (K562 WT); 3) with irradiated modified tumor-derived cells (K562 variant 1); 4) with irradiated modified tumor-derived cells (K562 variant 2); 5) with irradiated modified tumor-derived cells (K562 variant 2) in the absence of ZOL; and 6) with irradiated modified tumor-derived cells (K562 variant 2) in the absence of ZOL with re-stimulation on Days 7 and 14.

FIGS. 13B and 13C show expansion of both δ1 (left panel) and δ2 (right panel) T cells in donor 1 (FIG. 13B) and donor 2 (FIG. 13C).

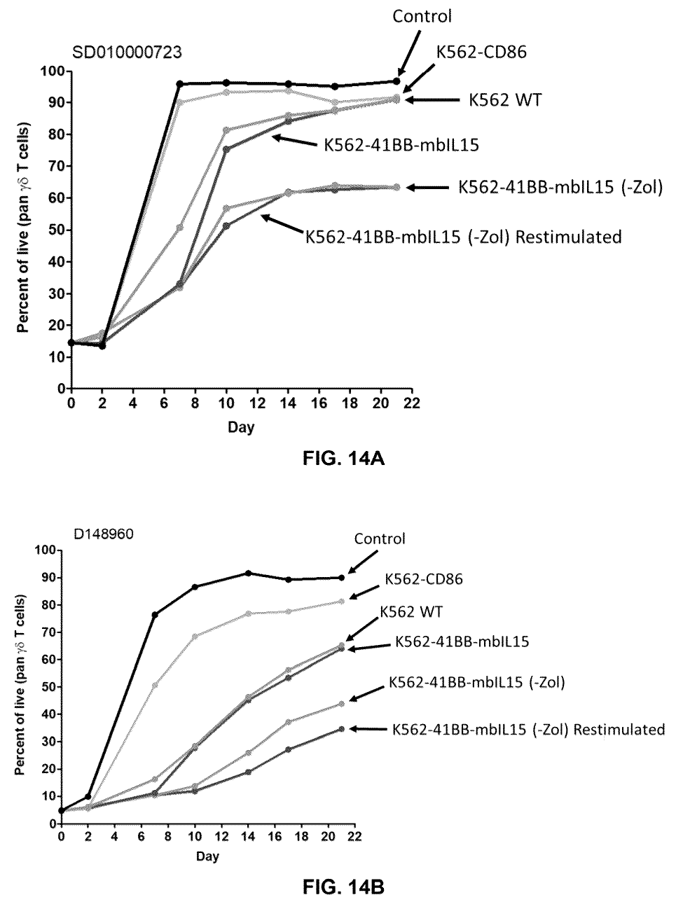

FIG. 14A and 14B show percentage of γδ T cells present within the entire live cell population in donor 1 (FIG. 14A) and donor 2 (FIG. 14B).

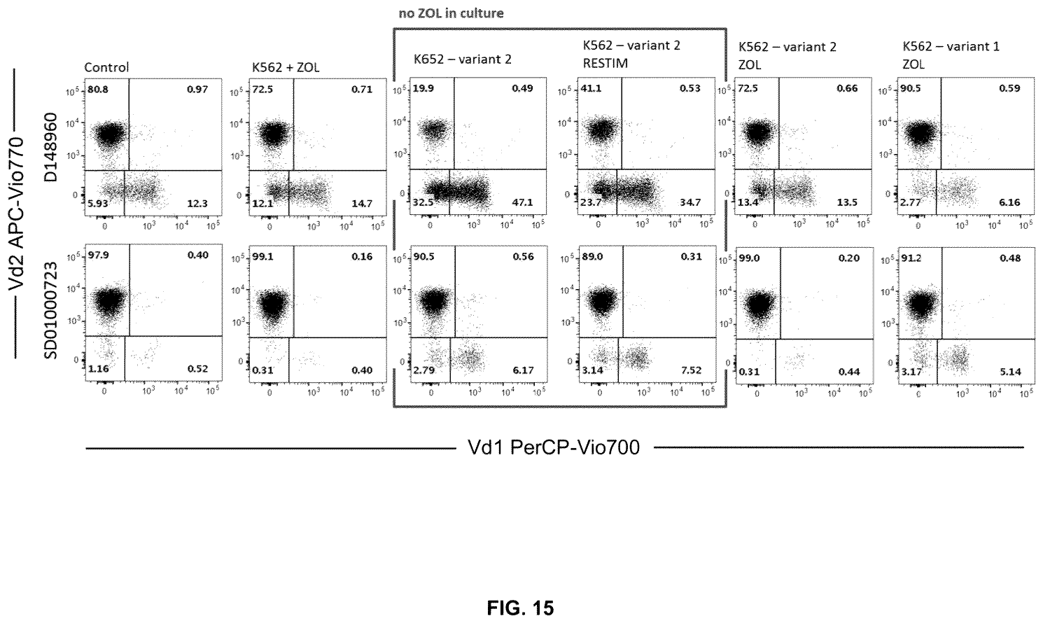

FIG. 15 shows that lack of zoledronate in the culture results in a polyclonal population (both δ1 and 82γδ T cells) compared to conditions in which zoledronate was in the culture. Cells were harvested on Day 21 and analyzed by flow cytometry to determine δ1 and δ2 populations.

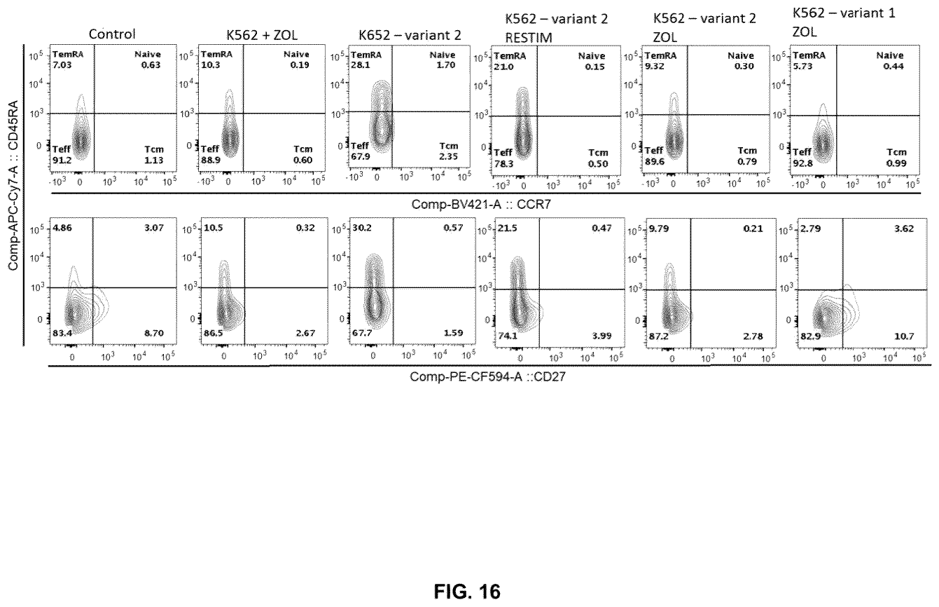

FIG. 16 shows that tumor-derived cell co-culture does not alter the memory phenotype of expanded γδ T cells. Cells were harvested on Day 21 and analyzed by flow cytometry to determine memory phenotype by detection of CD45, CD27, and CCR7 on the cell surface.

### Example 4

Re-stimulation of γδ T cells during expansion with allogenic cells leads to enhanced and prolonged expansion.

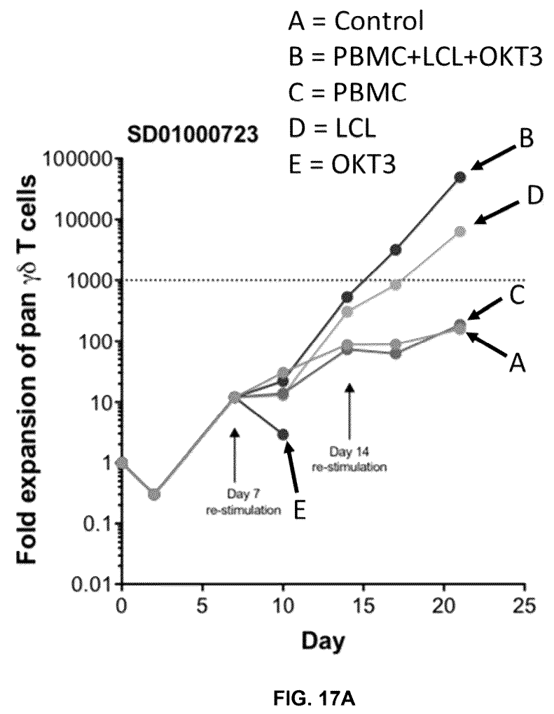

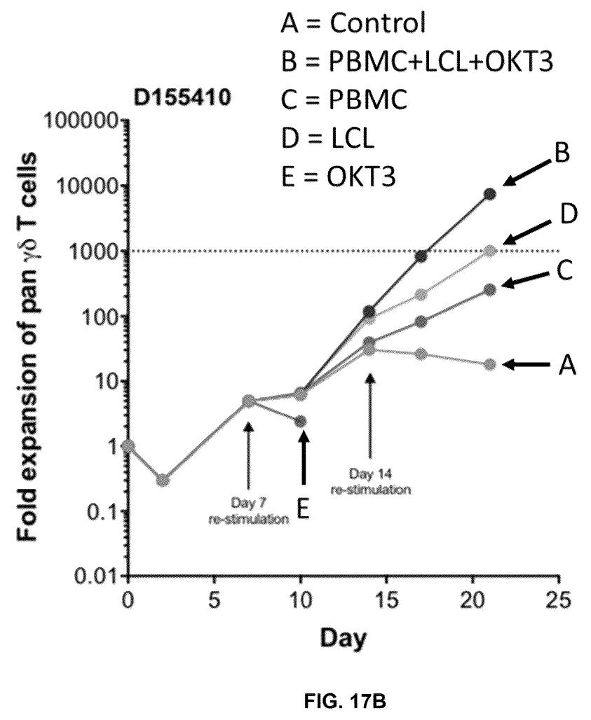

FIGS. 17A and 17B show the effect of re-stimulation with irradiated allogenic PBMC on the expansion of γδ T cells. Briefly, on Day 0, the αβ-TCR expressing T cells (including CD4+ and CD8+ T cells) depleted peripheral blood mononuclear cells (PBMC) (“γδ T cells”) were activated in the presence of zoledronate (ZOL) (5 μM), IL-2 (100 U/ml), and IL-15 (100 ng/ml). On Day 2, the activated γδ T cells were mock transduced. On Day 3, the mock-transduced cells were expanded. On Day 7, the expanded γδ T cells were separated into five separate groups to examine the effect of re-stimulation with allogenic feeder cells. Specifically, 2×10⁶ expanded γδ T cells were μlaced into each treatment group. The treatment groups were as follows: 1) IL-2+IL-15 (Control); 2) PBMC+LCL+OKT3+IL-2; 3) PBMC+IL-2; 4) LCL+IL-2; 5) OKT3+IL-2. For each group, PBMC =allogenic PBMCs pooled from 2-3 donors and irradiated and added in an amount of 25×10⁶ cells. LCL =irradiated lymphoblastoid cells and added in an amount of 5×10⁶ cells. OKT3 =soluble OKT3, an activating anti-CD3 antibody added in an amount of 30 ng/ml. IL-2 was added in an amount of 50 U/ml.

Each re-stimulation treatment was repeated on Day 14 and cells were harvested on Day 21 and analyzed.

FIG. 17A-B shows re-stimulation with allogenic PBMC and/or LCL increases fold-expansion without growth μlateau of γδ T cells obtained from two donors (D1 and D2) as compared with that without re-stimulation.

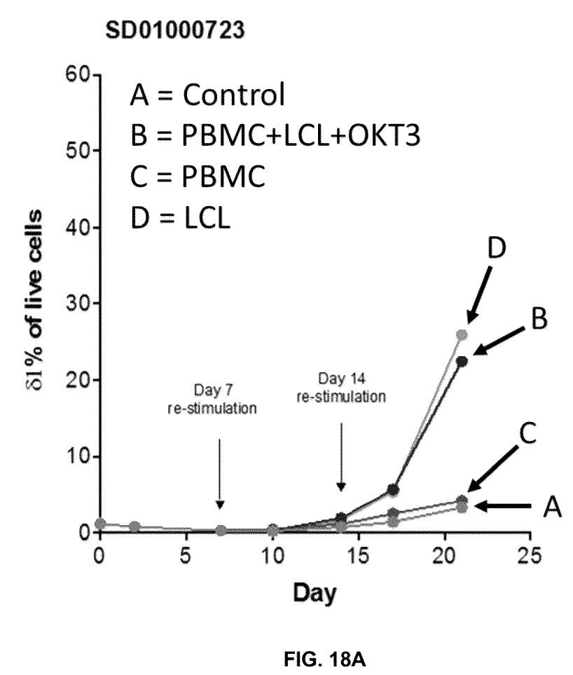

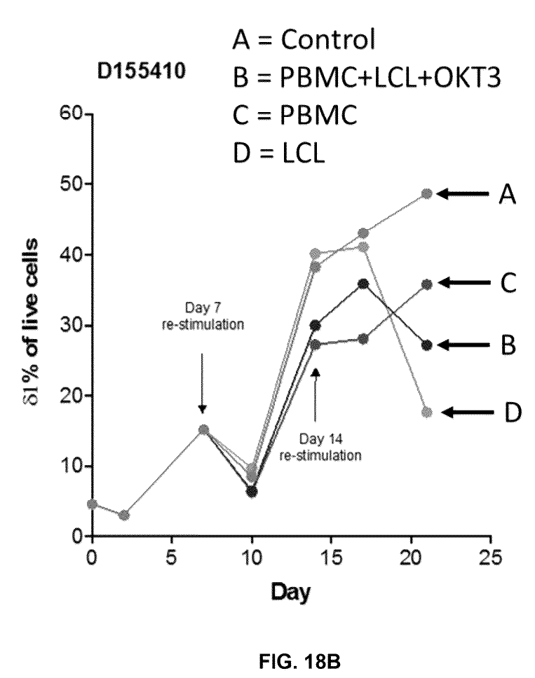

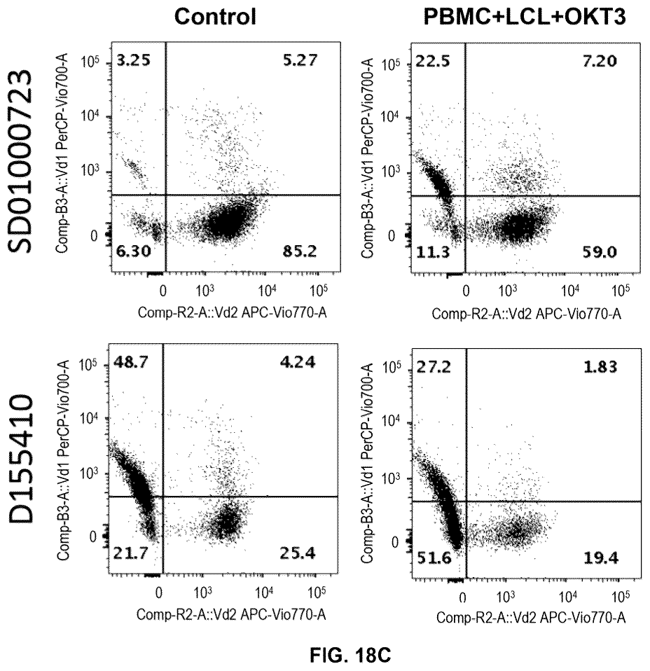

FIG. 18A-C shows re-stimulation with allogenic PBMC and/or LCL produces polyclonal (both δ1 and 82γδ T cells) population. The presence of δ1 cells as a percentage of live cells is shown for two donors in FIG. 18A & 18B. This data illustrate that presence of δ1 cells is donor dependent. FIG. 18C shows the results from control treatment (IL-2+IL-15) and from PBMC+LCL+OKT3 treatment (in the presence of IL-2) from the two donors on Day 21.

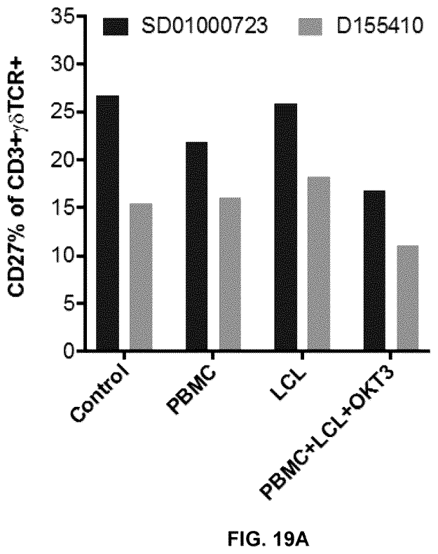

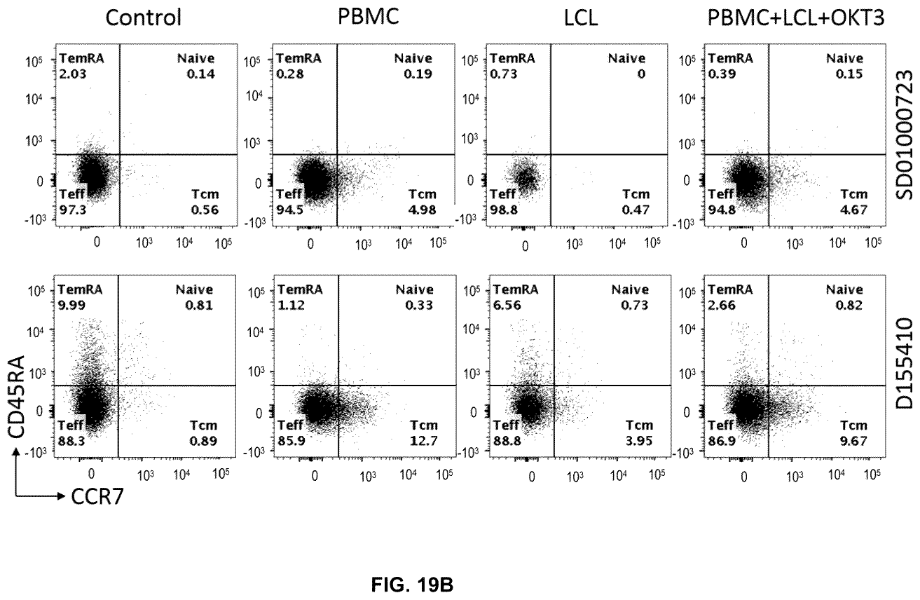

FIG. 19A-B shows the memory phenotype of expanded γδ T cell populations upon re-stimulation with PBMC and/or LCL. Memory phenotypes were measured on Day 14 instead of Day 21 and thus, were only re-stimulated once on Day 7. The expanded γδ T cell populations were analyzed by flow cytometry to determine memory phenotype by detection of CD45, CD27, and CCR7 on the cell surface. FIG. 19A presents CD27 detection on the expanded γδ T cell populations. There appears to be a slight decrease in the percentage of CD27 in expanded γδ T cells re-stimulated with PBMC+LCL+OKT3. FIG. 19B presents the CD45and CCR7 expression. An increased percentage of CCR7 is seen in expanded γδ T cells re-stimulated with PBMC and with PBMC+LCL+OKT3.

### Example 5

Generation of allogenic PBMCs pulsed with zoledronate for activation of γδ T cells.

As shown above in EXAMPLE 1, fresh autologous PBMCs pulsed with zoledronate (ZOL) and then irradiated can be used for re-stimulation of γδ T cells on Day 7 and optionally in additional re-stimulation steps (e.g., Day 14, etc.). However, this method requires several collections from the clinical donor.

To avoid the need for multiple collections, allogenic banks of PBMCs that are pulsed with ZOL can be generated for use in the one or more re-stimulations. These allogenic banks of PBMCs were generated as follows: frozen allogenic PBMCs (including αβ T cells) collected from the donor were thawed and pulsed with 100 μM ZOL for 4 hours. These ZOL-treated allogenic PBMCs were then washed and frozen. The frozen vials containing the ZOL-treated allogenic PBMCs were irradiated at 50 Gy and stored for future use. These irradiated, ZOL-treated allogenic PBMCs were thawed for re-stimulation at Day 7 of the manufacturing process.

### Example 6

Peptide-Specific Killing Activity of Transduced γδ T Cells

Transduced γδ T cells were prepared by the expansion methods shown in Table 3.

TABLE 3

| | Process 1 | Process 2 | Process 3 | Control |
|---|---|---|---|---|
| Feeders | irradiated | Zoledronate | Pooled | None |
| | K562-41BBL- | pulsed | irradiated | |
| | mbIL15 (i.e., | irradiated | allogenic | |
| | K562 cells | allogenic | PBMCs (2-3 | |
| | expressing | PBMCs | donors) + | |
| | membrane | | LCLs + OKT3 | |
| | bound IL15 and | | | |
| | 4-1 BB Ligand) | | | |
| Feeders | Day 0 | Day 7 and | Day 7 and | None |
| added to the | | Day 14 | Day 14 | |
| culture on | | | | |
| Feeders:γδ T | 2 K562:1 total | 20 PBMC:1 γδ | 1 γδ T cells:2.5 | None |
| cells ratio | cells (PBMC + | T cells ratio | LCL : 12.5 | |
| | γδ T cells) ratio | | PBMC ratio | |
| Cytokines | cells grown in | cells grown in | cells grown in | cells grown in |
| | IL15 + IL2 | IL15 + IL2 | IL15 + IL2 for | IL15 + IL2 |
| | throughout 21 | throughout 21 | first 7 days and | throughout 21 |
| | Day | Day | then switched | Day |
| | manufacturing | manufacturing | to IL2 only after | manufacturing |
| | | | Day 7 up to | |
| | | | Day 21 of | |
| | | | manufacturing | |

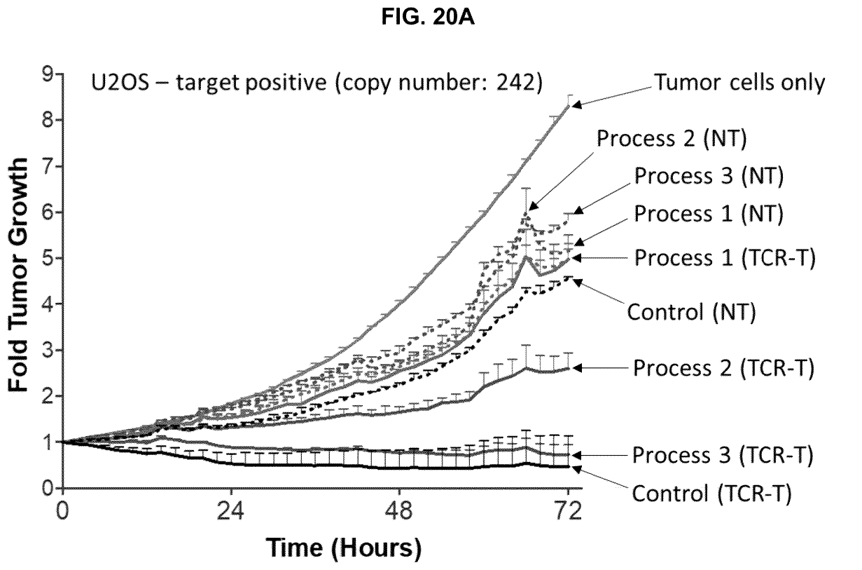

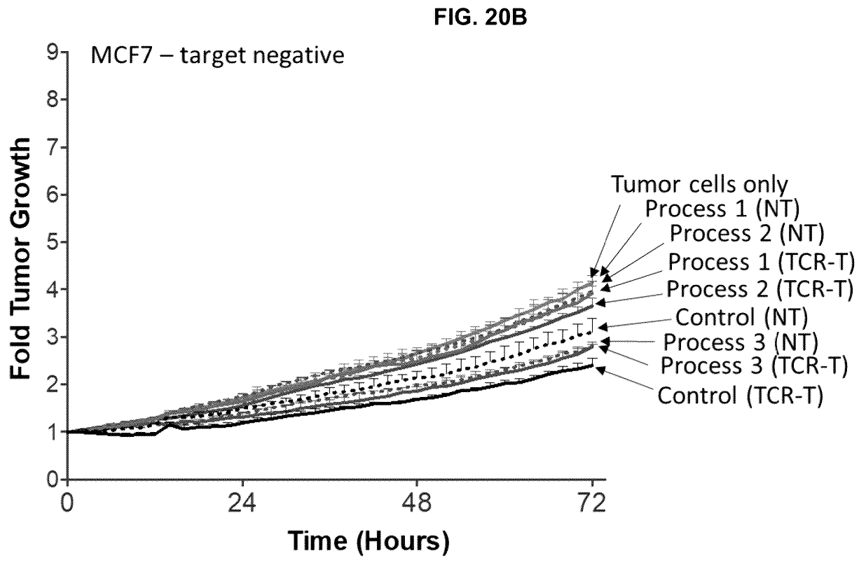

The above processes generated 6.8% peptide/MHC-specific TCR-transduced γδ T (Tet+) cells from Process 1, 21.9% Tet+ cells from Process 2, 47.4% Tet+ cells from Process 3, and 28.8% Tet+ cells from Control. To determine the peptide-specific killing activity of γδ T cells transduced with TCR (TCR-T), effector T cells, i.e., γδ T cells expanded by Process 1, 2, 3, or Control, were co-cultured with tumor cells (e.g., peptide-positive U2OS cells, which may present about 242 copies per cell, and peptide-negative MCF-7 cells) at a 3:1 (effector cell: tumor cell) ratio. Non-transduced γδ T cells (NT) serve as negative controls. Tumor cell viability/death was analyzed in real time using the Incucyte live-cell analysis system. FIG. 20A shows, against peptide-positive U2OS cells, the killing activity of γδ T cells (TCR-T) expanded by Process 3 is significantly higher than that expanded by Process 1 or Process 2 and is similar to that expanded by Control (TCR-T). γδ TCR-T cells expanded by Process 2, Process 3, and Control show higher killing activity than their respective γδ NT cells. It appears no significant difference between the killing activities of γδ TCR-T cells and γδ NT cells expanded by Process 1. FIG. 20B shows, against peptide-negative MCF-7, the killing activities of γδ T cells (TCR-T) expanded by various processes appear similar to that of their respective non-transduced γδ T cells (NT) cells. These results suggest that TCR-transduced γδ T cells expanded by Process 2, Process 3, and Control can recognize and kill tumor cells in a peptide-specific manner.

### Example 7

### Optimization of γδ T Cell Manufacturing

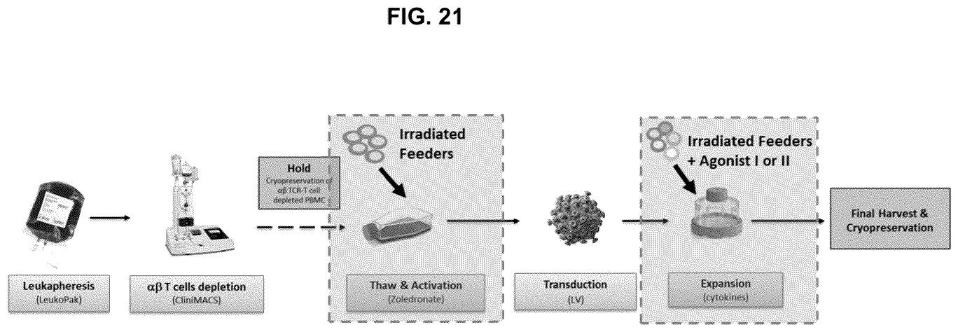

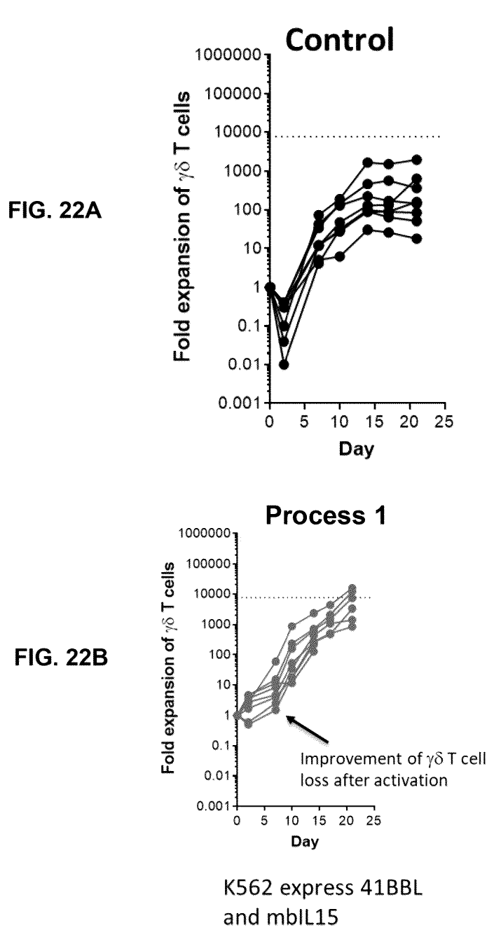

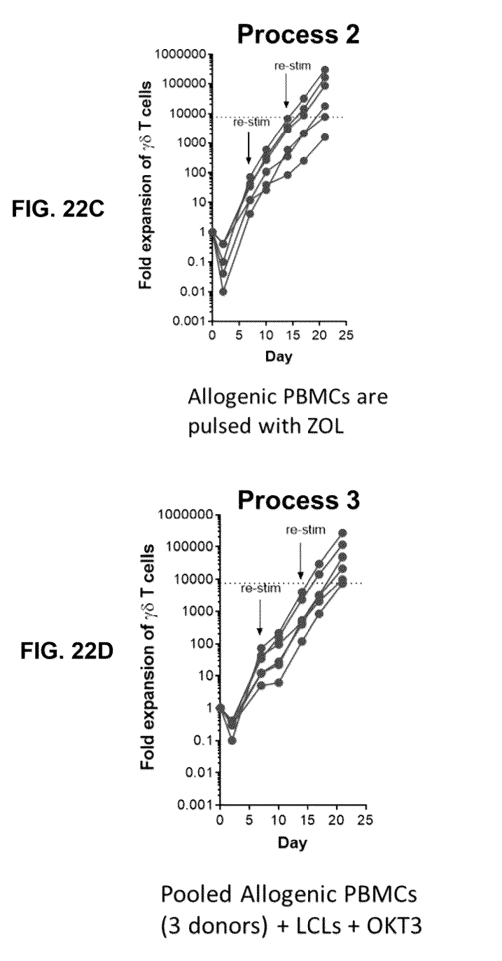

FIG. 21 shows γδ T cell manufacturing process, e.g., the control and Processes 1-3 (Table 3), in which cells may be thawed, activated, and/or expanded in the presence of feeder cells and/or agonists I or II, e.g., anti-CD3, anti-CD28, anti-41BB, anti-ICOS, anti-CD40, and anti-OX40 antibodies. Feeder cells were added on Day 0 (Process 1) or Day 7 (re-stim) and Day 14 (re-stim) (Process 2 and Process 3). FIGS. 22A-22D show growth μlateau observed in γδ T cells prepared by the control process (without feeder) (FIG. 22A) was overcome by feeder cell stimulation (e.g., Process 1 (FIG. 22B), Process 2 (FIG. 22C), and Process 3 (FIG. 22D)). Loss of γδ T cells after activation observed in cells produced by the control process, Process 2, and Process 3 was improved in cells produced by Process 1. On the other hand, γδ T cells produced by Process 2 and Process 3 exhibit higher fold expansion than that produced by Process 1. γδ T cells produced by Process 3 achieved at least 10,000-fold expansion.

### γδ T Cells Produced by Process 3 Exhibit “Younger” T Cell Phenotype

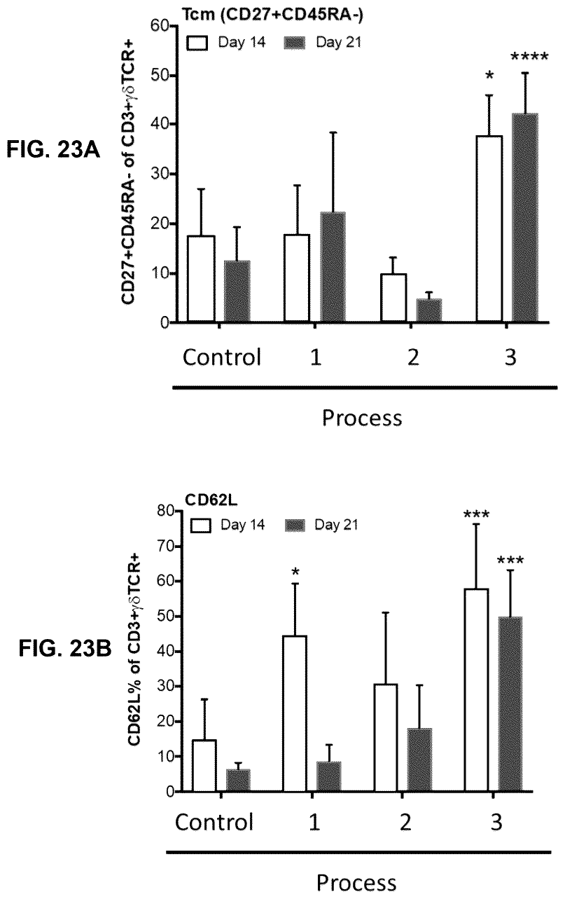

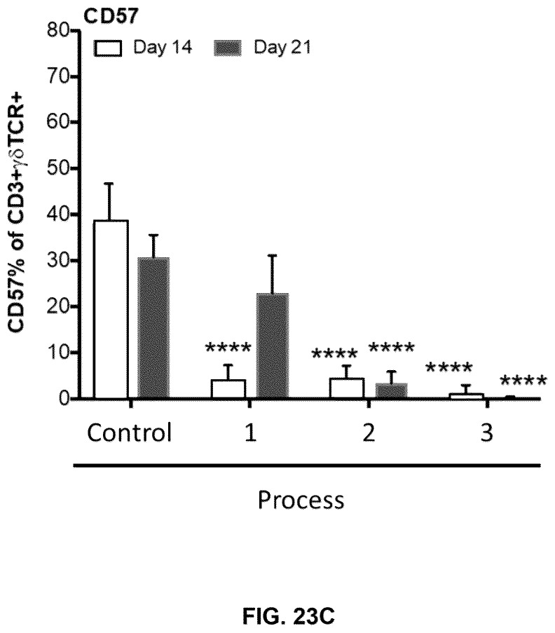

Phenotypes of γδ T cells produced by the control process and Processes 1-3 were analyzed. FIG. 23A shows γδ T cells produced by Process 3 at Day 14 and Day 21 have more % of γδ T cells exhibiting Tcm phenotype, e.g., CD27+CD45RA−, than those produced by the control process, Process 1, and Process 2. Consistently, γδ T cells produced by Process 3 at Day 14 and Day 21 have more % of γδ T cells exhibiting Tcm phenotype, e.g., CD62L+ (FIG. 23B), and less % of γδ T cells exhibiting non-Tcm phenotype, e.g., CD57+ (FIG. 23C), than those produced by the control process, Process 1, and Process 2. (n=4; mean+SD; ANOVA with Tukey's post hoc compared to control; ****p<0.0001; ***p<0.001; **p<0.01; *p<0.5)

### Effect on Immune Checkpoint Protein Expression in γδ T Cells Produced by Various Processes

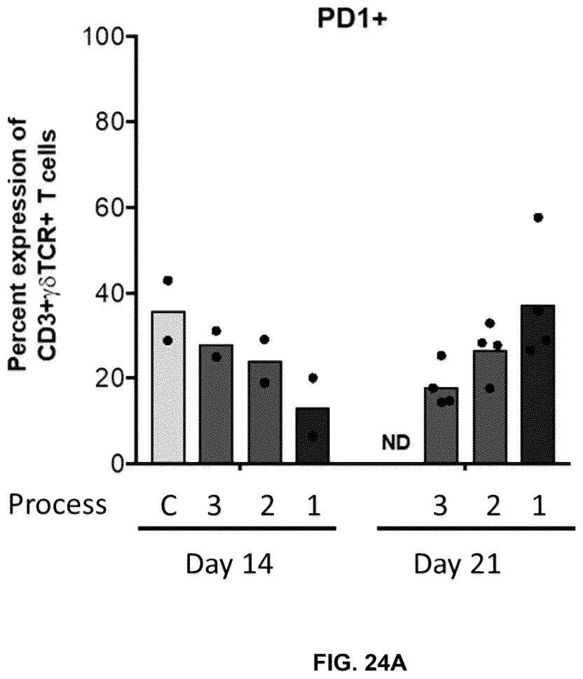

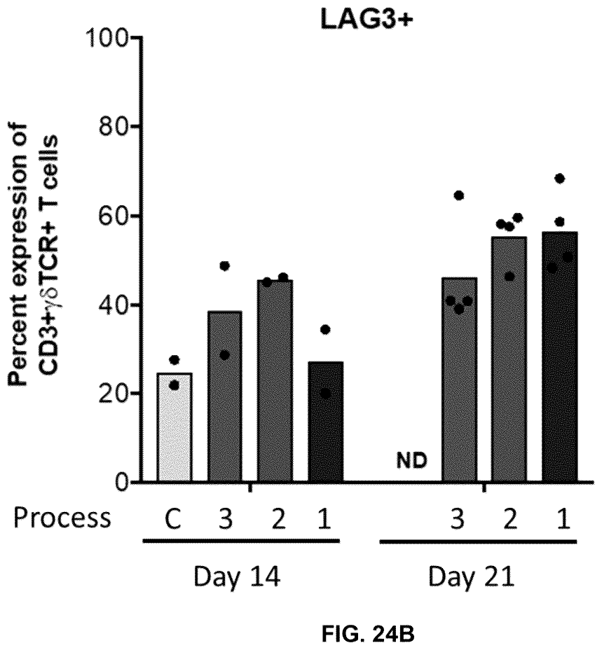

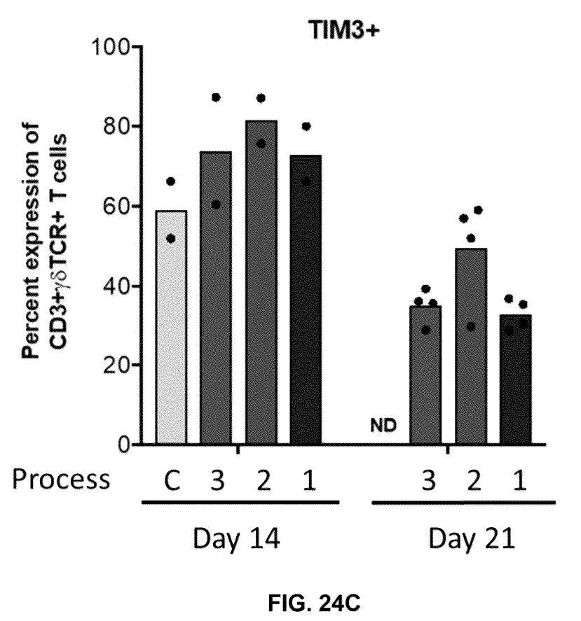

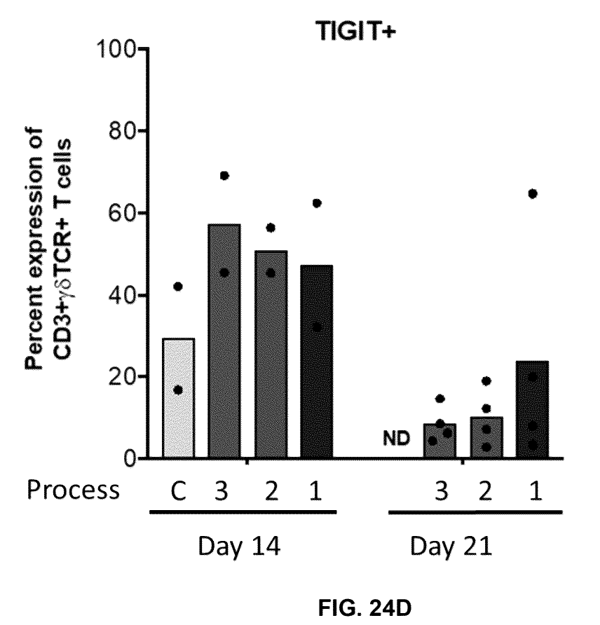

To determine immune checkpoint protein expression in γδ T cells produced by various processes, % PD1+ (FIG. 24A), LAG3+ (FIG. 24B), TIM3+ (FIG. 24C), and TIGIT+ (FIG. 24D) γδ T cells were determined. FIG. 24A shows, at Day 14, % PD1+ γδ T cells produced by Processes 1-3 decreases as compared with that produced by the control process (C). On the other hand, % PD1+ γδ T cells produced by Process 1 increases from Day 14 to Day 21. % PD1+ γδ T cells produced by Process 2 and Process 3 seems comparable from Day 14 to Day 21. FIG. 24B shows % LAG3+ γδ T cells produced by Processes 2 and 3 increases as compared with that produced by the control process (C) at Day 14. While % LAG3+ γδ T cells produced by Process 2 and Process 3 seem comparable from Day 14 to Day 21, % LAG3+ γδ T cells produced by Process 1 increases from Day 14 to Day 21. FIG. 24C shows % TIM3+ γδ T cells produced by Processes 1-3 decrease from Day 14 to Day 21. FIG. 24D shows % TIGIT+ γδ T cells produced by Processes 1-3 decrease from Day 14 to Day 21.

### Effect on Transgene Expression of γδ T Cells Produced by Various Processes

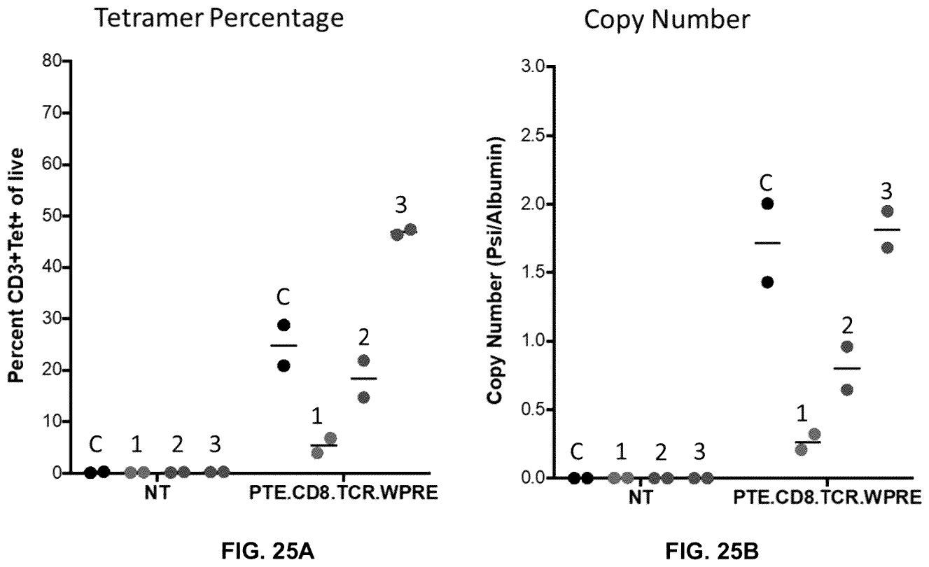

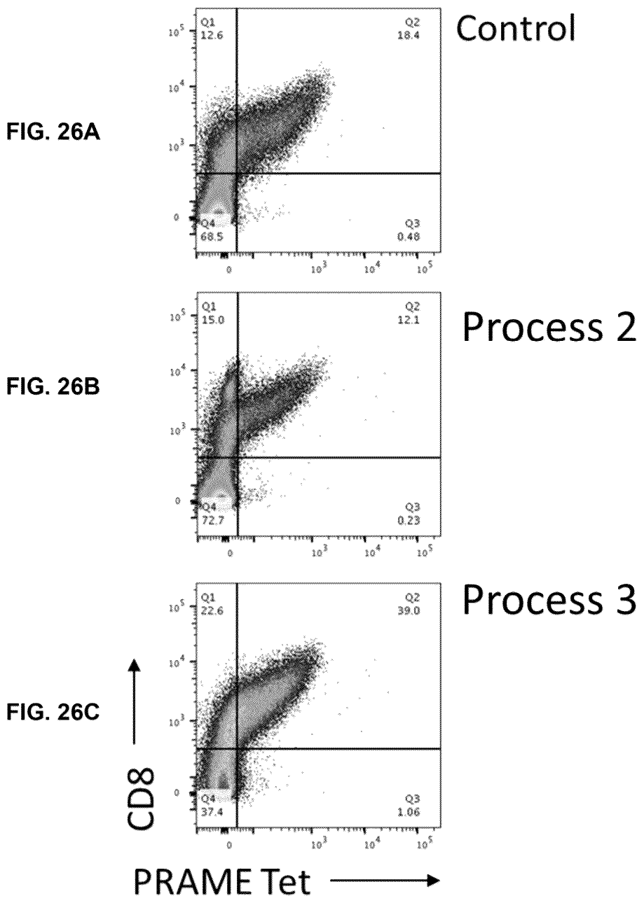

γδ T cells produced by Processes 1-3 and the control process (C) were transduced with viral vector encoding CD8αβ and TCRαβ (PTE.CD8.TCR.WPRE) followed by target peptide (PRAME)/MHC tetramer (Tet) staining. FIG. 25A shows that, at Day 14 after the first re-stimulation, % Tet+ γδ T cells transduced with PTE.CD8.TCR. WPRE produced by Process 3 is higher than that produced by Process 1, Process 2, and the control process. The non-transduced (NT) cells serve as negative controls. γδ T cells transduced with PTE.CD8.TCR.WPRE produced by Process 3 yielded more CD8+ PRAME Tet+ γδ T cells (39%, FIG. 26C) than that produced by the control process (18.4% FIG. 26A) and by Process 2 (12.1%, FIG. 26B). MFI is similar among transduction conditions. FIG. 25B shows that copy number of transgene incorporated in γδ T cells produced by Process 3 is about 2 copies/cell, which is comparable to that produced by the control process and is higher than that produced by Process 1 and Process 2.

### Effect of Initial K562 Stimulation in Process 1 on Transduction and Expansion

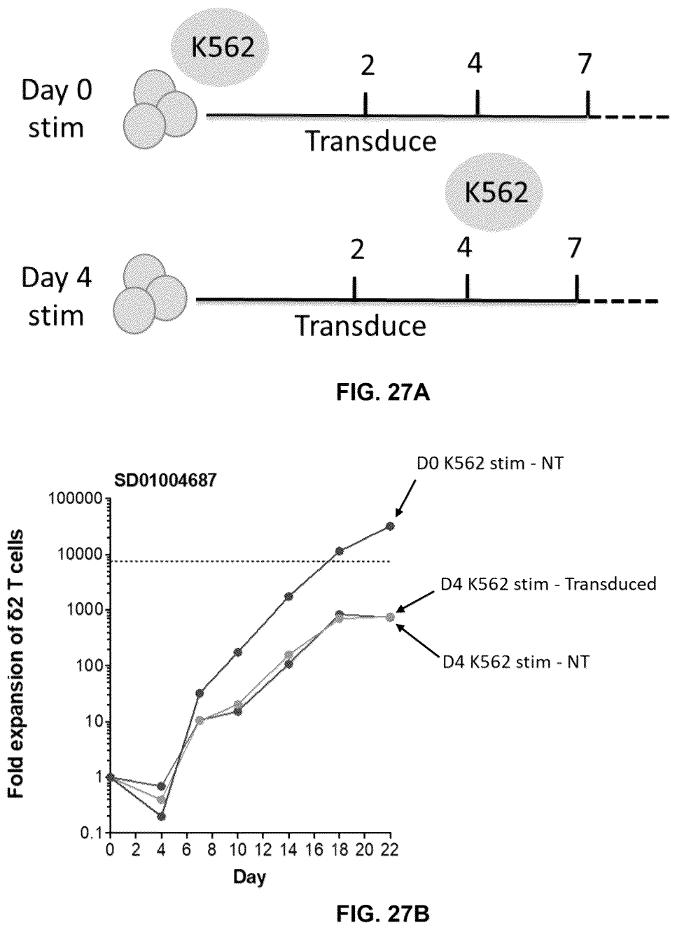

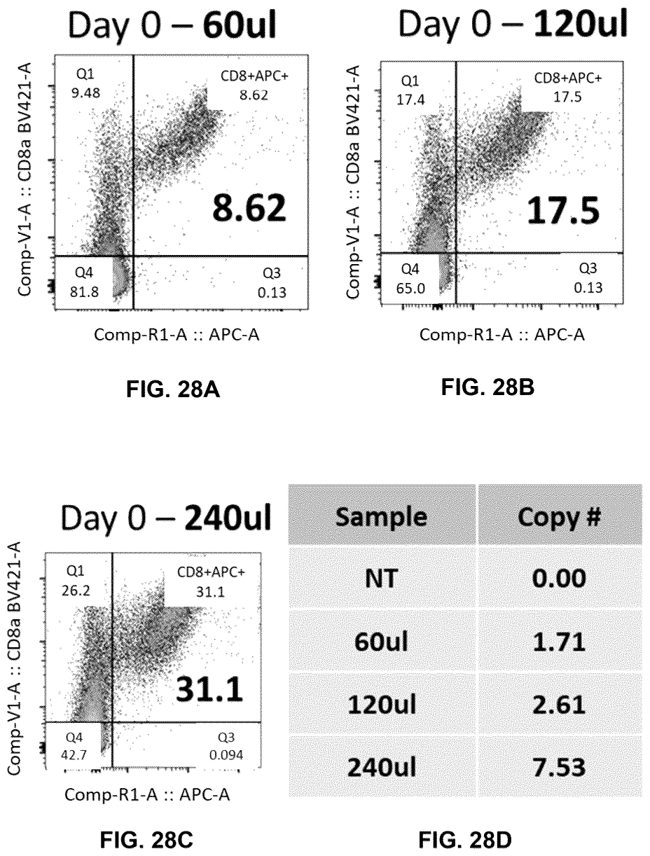

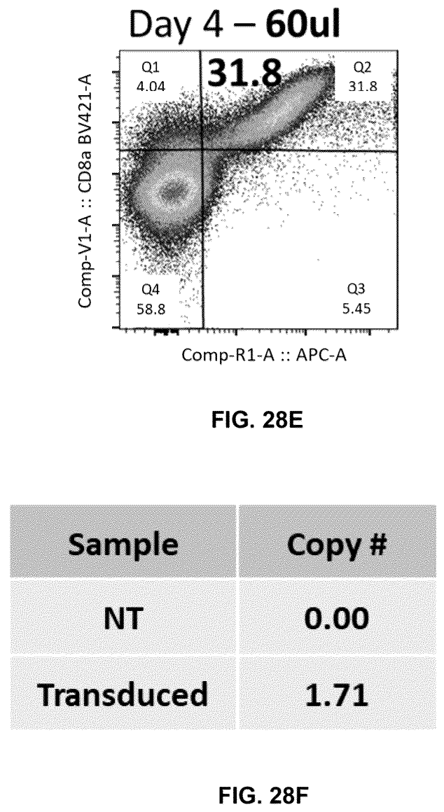

To determine the effect of initial K562 stimulation on γδ T cell products prepared by Process 1, as shown in FIG. 27A, γδ T cells were stimulated on Day 0 prior to transduction with PTE.CD8.TCR.WPRE on Day 2 or γδ T cells were stimulated on Day 4 after transduction with PTE.CD8.TCR.WPRE on Day 2. FIG. 27B shows fold expansion of γδ T cells stimulated on Day 4 with or without transduction is lower than that stimulated on Day 0. FIGS. 28A-28C show, for γδ T cells stimulated with K562 cells on Day 0, γδ T cells transduced with 60 μl, 120 μl, and 240 μl of PTE.CD8.TCR.WPRE yielded 8.62%, 17.5%, and 31.1% of CD8+PRAME Tet+ cells, respectively. FIG. 28D shows the copy numbers of the integrated transgene. Although γδ T cells transduced with 240 μl of PTE.CD8.TCR.WPRE yielded 31.1% of CD8+PRAME Tet+ cells (FIG. 28C), the copy number of the integrated transgene is 7.53 copies/cell, which exceeds the 5 copies/cell safety limit. In contrast, FIG. 28E shows γδ T cells transduced with 60 μl of PTE.CD8.TCR.WPRE followed by stimulation with K562 cells on Day 4 yielded 31.8% of CD8+PRAME Tet+ cells with the copy number of the integrated transgene of 1.71 copies/cell. These data suggest that, while K562 stimulation on Day 4 after transduction may allow sufficient transduction resulting in better and safer T cell products than that stimulated on Day 0 prior to transduction, it may limit expansion.

### Effect of Re-Stimulations on Transgene Expression

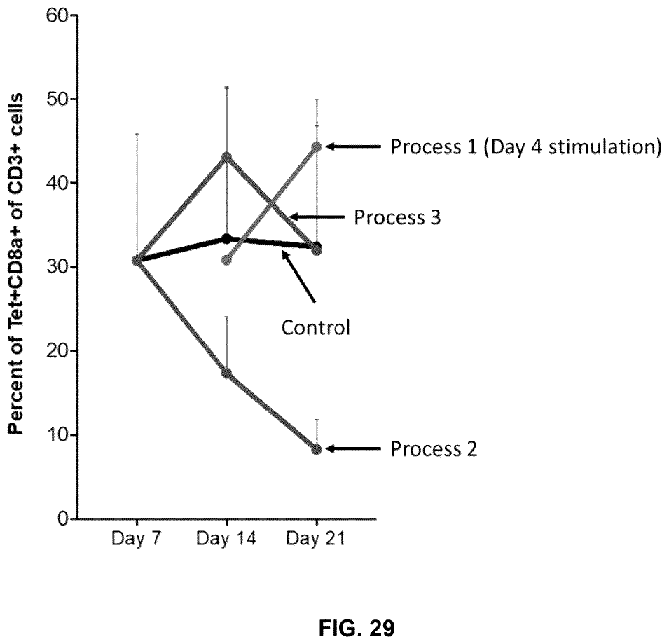

FIG. 29 shows transgene (PTE.CD8.TCR.WPRE) expression, e.g., % CD8+PRAME Tet+ γδ cells (1) increases from Day 14 to Day 21 for cells produced by Process 1 (n=2) with stimulation on Day 4; (2) decreases from Day 7 to Day 21 for cells produced by Process 2 (n=4) with re-stimulation on Day 7 and Day 14; and (3) increases from Day 7 to Day 14 and then decreases from Day 14 to Day 21 for cells produced by Process 3 (n=4) with re-stimulation on Day 7 and Day 14. Transgene expression remains at similar levels for cells produced by the control process.

### Effect on Functions of γδ T Cells Produced By Various Processes

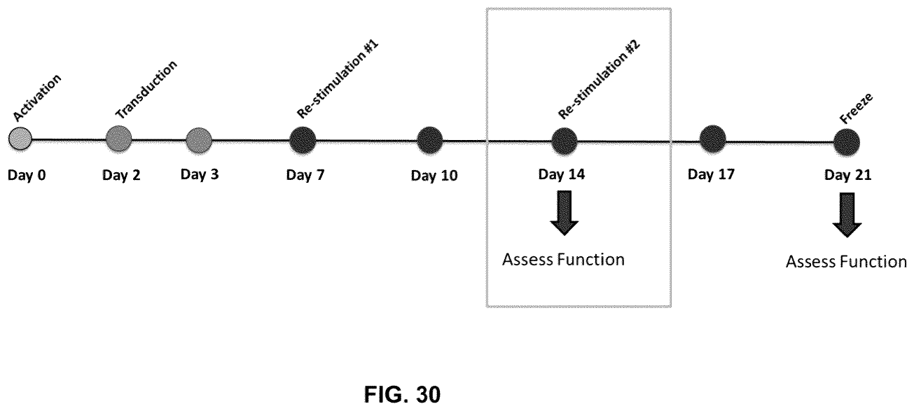

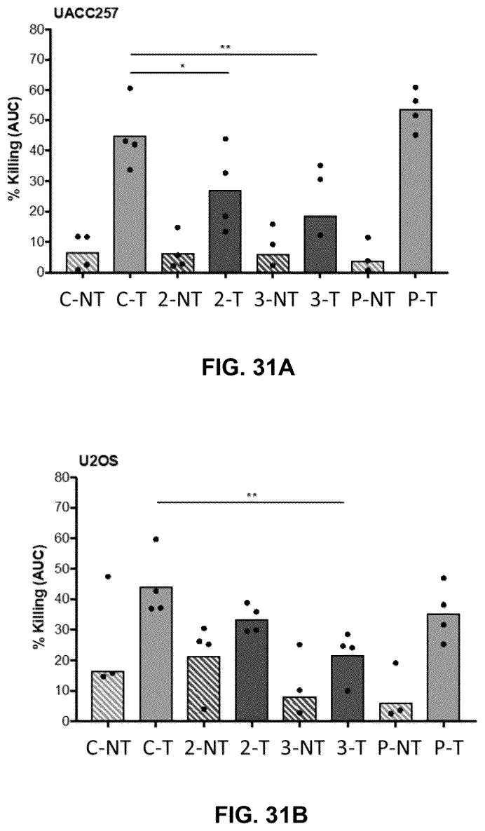

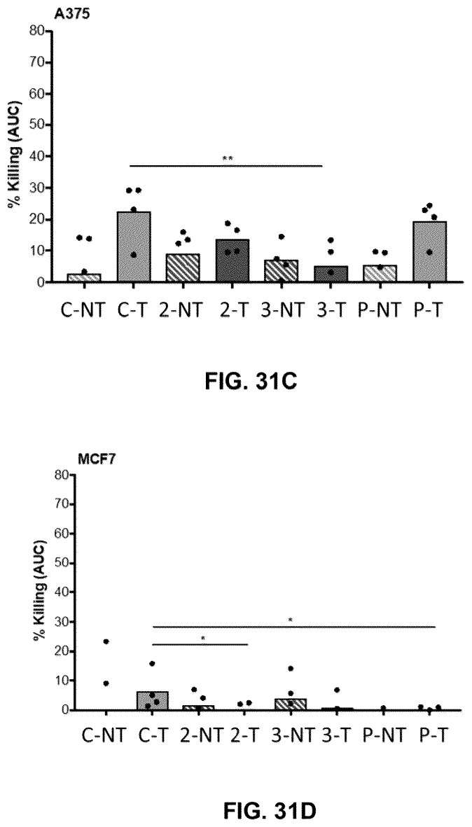

FIG. 30 shows functional assessment performed on Day 14 after the first re-stimulation on Day 7. γδ T cells produced by Processes 2 and 3 and the control process (C) were transduced with PTE.CD8.TCR.WPRE (2-T, 3-T, and C-T, respectively) or without transduction (2-NT, 3-NT, and C-NT, respectively). CD8+ [symbol] [symbol] T cells transduced with the same TCR or without transduction serve as positive controls (P-T and P-NT). Cells thus prepared were incubated with target cells, e.g., UACC257 (˜1081 target peptides per cell), U2OS (˜242 target peptides per cell), A375 (˜51 target peptides per cell), and MCF-7 (0target peptides per cell), at an effector/target ratio of 3:1, followed by cytotoxicity assay. Effector cells were normalized to transduction efficiency. FIGS. 31A-31C show, after the first re-stimulation, cytolytic activities of γδ T cells produced by Process 2 (2-T) and Process 3 (3-T) are lower than that of C-T and P-T against UACC257, U2OS, and A375 cells, respectively. FIG. 31D shows minimum cytolytic activities of γδ T cells produced by Process 2 (2-T) and Process 3 (3-T) against the non-target MCF7 cells. (ANOVA with Tukey's post hoc compared to control; n=4 donors; **p<0.01; *p<0.5)

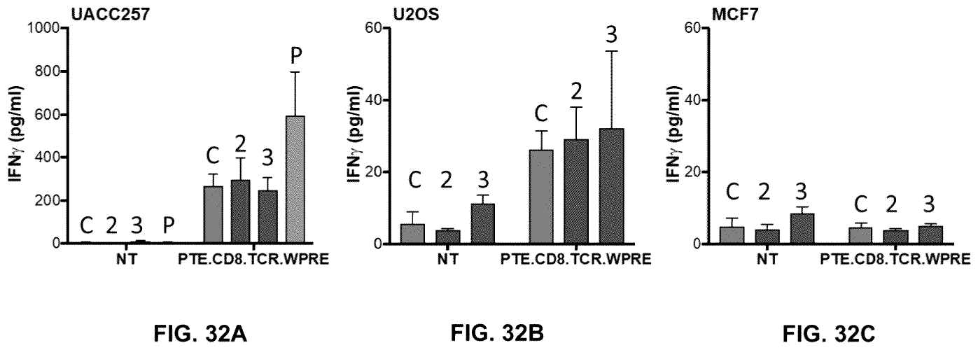

FIGS. 32A and 32B show, after the first re-stimulation, IFNγ secretion from γδ T cells produced by Process 2 (2) and Process 3 (3) are comparable to that produced by the control process (C) against UACC257 and U2OS cells, respectively, at an effector/target ratio of 3:1. Effector cells were normalized to transduction efficiency. FIG. 32C shows minimum IFNγ secretion from γδ T cells produced by Process 2 (2) and Process 3 (3) against the non-target MCF7 cells. The non-transduced (NT) cells serve as negative controls. CD8+ [symbol] [symbol] T cells transduced with the same TCR serve as positive controls (P). (n=2 donors; 2technical replicates/donor)

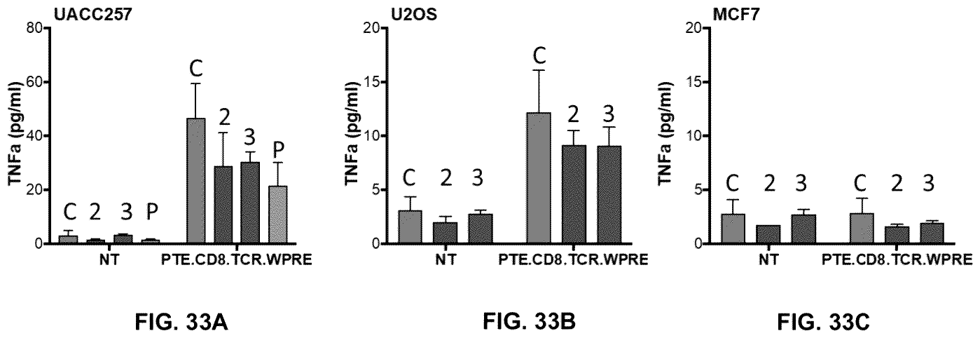

FIGS. 33A and 33B show, after the first re-stimulation, TNFα secretion from γδ T cells produced by Process 2 (2) and Process 3 (3) decrease as compared with that produced by the control process (C) against UACC257 and U2OS cells, respectively, at an effector/target ratio of 3:1. Effector cells were normalized to transduction efficiency. FIG. 33C shows minimum TNFα secretion from γδ T cells produced by Process 2 (2) and Process 3 (3) against the non-target MCF7 cells. The non-transduced (NT) cells serve as negative controls. CD8+ [symbol] [symbol] T cells transduced with the same TCR serve as positive controls (P). (n=2 donors; 2 technical replicates/donor)

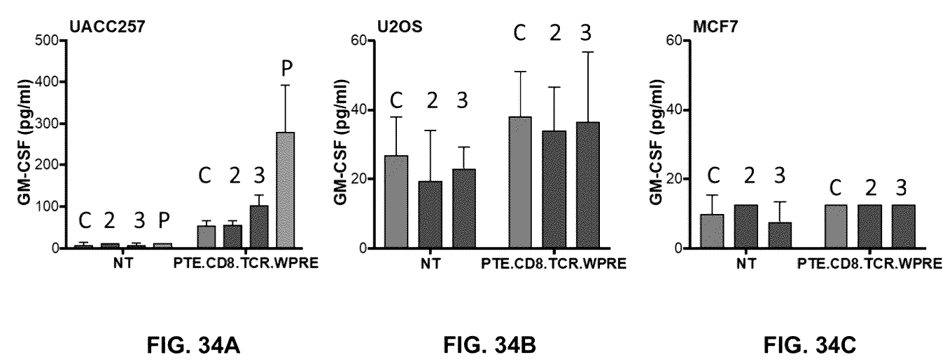

FIG. 34A shows, after the first re-stimulation, GM-CSF secretion from γδ T cells produced by Process 3 (3) increases as compared with that produced by Process 2 (2) and the control process (C) against UACC257 at an effector/target ratio of 3:1. Effector cells were normalized to transduction efficiency. FIG. 34B shows this increase of GM-CSF was not observed against U2OS cells, which express lower number of target peptide. FIG. 34C shows minimum GM-CSF secretion from γδ T cells produced by Process 2 (2) and Process 3 (3) against the non-target MCF-7 cells. The non-transduced (NT) cells serve as negative controls. CD8+ [symbol] [symbol] T cells transduced with the same TCR serve as positive controls (P). (n=2 donors; 2 technical replicates/donor) Furthermore, no differences were observed between non-transduced cells and transduced cells in the expression levels of IL-6, perforin, and granzyme B. Other analytes tested but below limit of detection include IL-2, IL-4, IL-5, IL-10, IL-12p70, and IL-17a.

### Tumor Cell Killing By γδ T Cells Produced by Various Processes

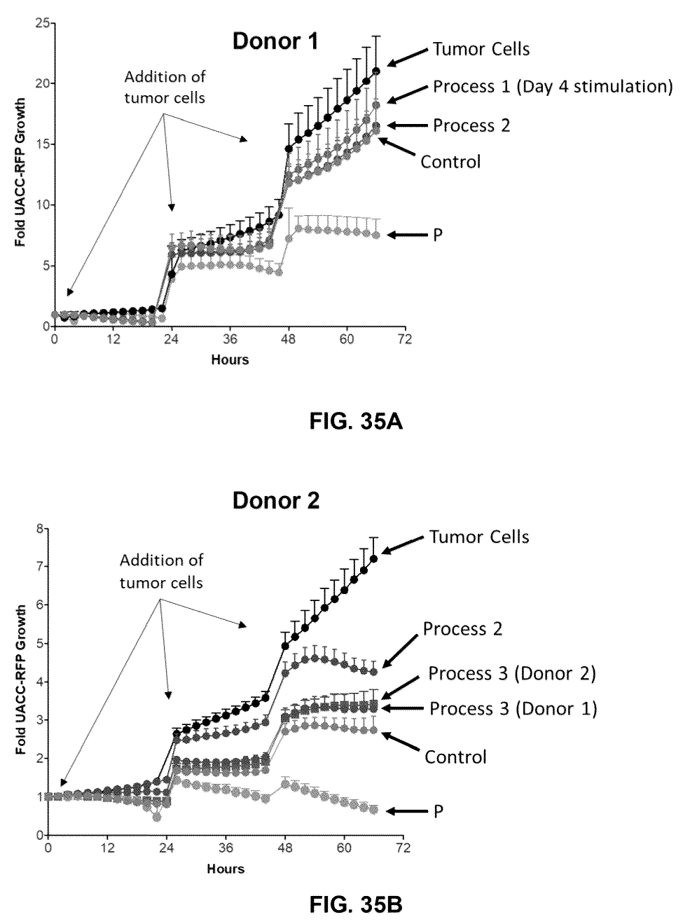

Tumor cell killing assays were performed at an effector/target ratio of 5:1. Effector cells were normalized to transduction efficiency using UACC257 cells (˜1081 target peptides per cell). UACC257 cells were added to the assays at three different time points, as indicated. FIG. 35A shows UACC257 tumor cell growth is inhibited by γδ T cells obtained from Donor 1 produced by Process 1 (Day 4 stimulation), Process 2, and the control process. CD8+ [symbol] [symbol] T cells transduced with the same TCR serve as positive controls (P). FIG. 35B shows UACC257 tumor cell growth is inhibited by γδ T cells obtained from Donor 2 produced by Process 2, Process 3, and the control process. CD8+ [symbol] [symbol] T cells transduced with the same TCR serve as positive controls (P).

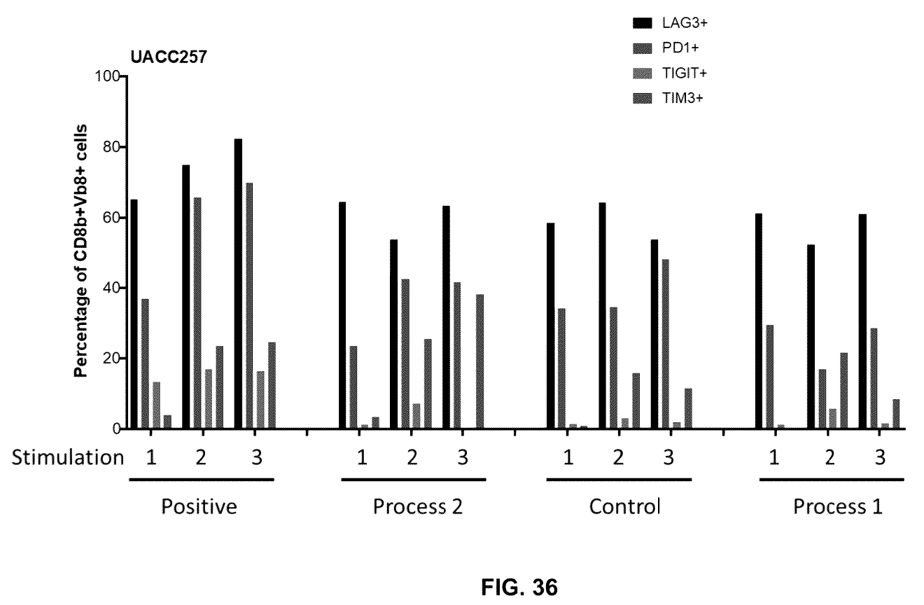

The expression of immune checkpoint molecules, e.g., LAG3, PD-1, TIGIT, and TIM3, in γδ T cells transduced with PTE.CD8.TCR.WPRE produced by various processes after up to 3× tumor stimulations (1, 2, and 3) were determined. FIG. 36 shows the expression of LAG3, PD-1, TIGIT, and TIM3 appear comparable among γδ T cells produced by Process 1, Process 2, and the control process. CD8+ [symbol] [symbol] T cells transduced with the same TCR serve as positive controls (Positive).

### Example 8

### Effect of Histone Deacetylase Inhibitors (HDACi) and IL-21 on γδ T Cell Manufacturing

Wang et.al. show that HDACi and IL21 can cooperate to reprogram human effector CD8+ T cells to memory T cells. (Cancer Immunol Res Jun. 1 2020 (8) (6) 794-805; the content of which is hereby incorporated by reference in its entirety). For example, pretreating tumor-infiltrating lymphocytes with HDACi, e.g., suberoylanilide hydroxamic acid (SAHA) or panobinostat (Pano), in the presence of IL-21 can increase Tcm αβ T cells (CD28+CD62L+) after 2 weeks of culture.

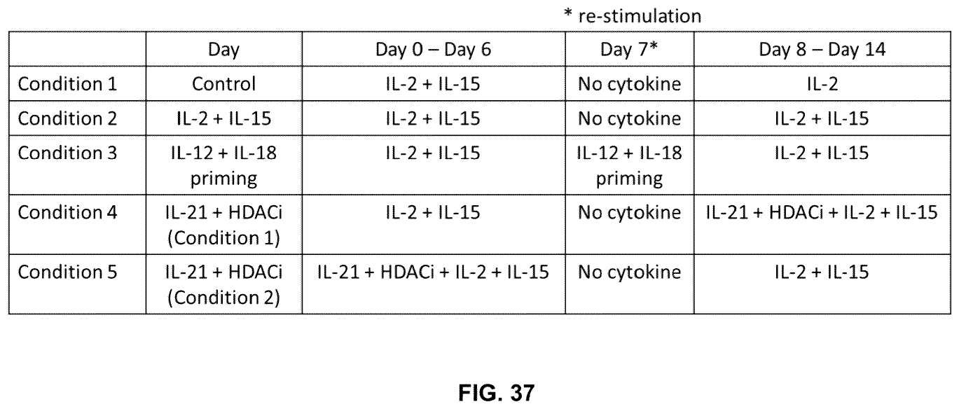

To test the effect of HDACi+IL-21 on the T cell products prepared by Process 3 feeder cells, FIG. 37 shows experimental design, e.g., under Condition 4, γδ T cells may be activated in the presence of zoledronate+IL-2+IL-15 on Day 0, expanded in the presence of IL-2+IL-15 from Day 0 to Day 6, followed by re-stimulation by Process 3 feeder cells in the absence of cytokines on Day 7, followed by expansion in the presence of HDACi+IL-21+IL-2+IL-15 from Day 8 to Day 14. Under Condition 5, γδ T cells may be activated in the presence of zoledronate+IL-2+IL-15 on Day 0, expanded in the presence of HDACi+IL-21+IL-2+IL-15 from Day 0 to Day 6, followed by re-stimulation by Process 3 feeder cells in the absence of cytokines on Day 7, followed by expansion in the presence of IL-2+IL-15 from Day 8 to Day 14.

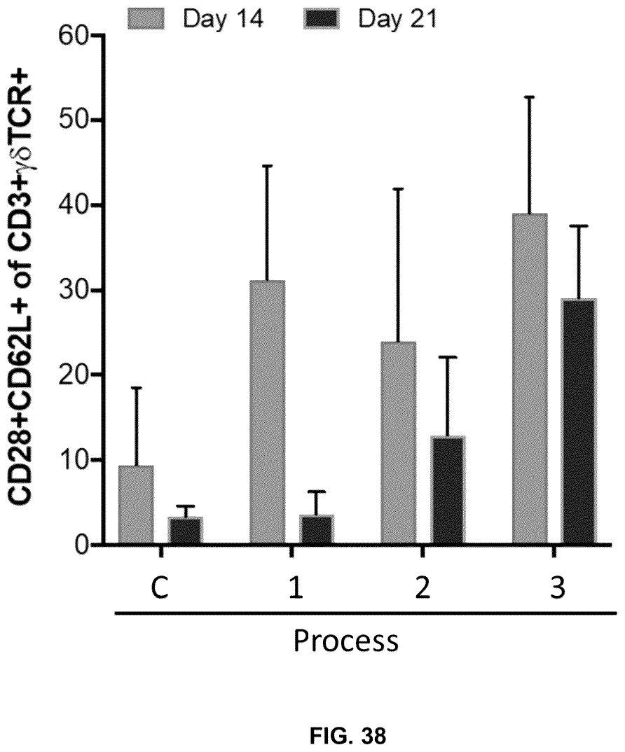

FIG. 38 shows, in the absence of HDACi and IL-21, re-stimulation by Process 3 feeder cells (pooled irradiated allogenic PBMCs+LCLs+OKT3) on Day 7 and on Day 14 resulted in more CD28+CD62L+γδ T cells at Day 14 and Day 21 than that re-stimulated by Process 1 feeders cells (irradiated K562-41BBL-mbIL15), Process 2 feeder cells (zoledronate pulsed irradiated allogenic PBMCs), and the control process (no feeder cells). The amount of CD28+CD62L+ γδ T cells decreases after the second re-stimulation on Day 14 for all processes. (n=4; mean+SD; ANOVA with multiple comparisons compared to control; ***p<0.0005; *p<0.5)

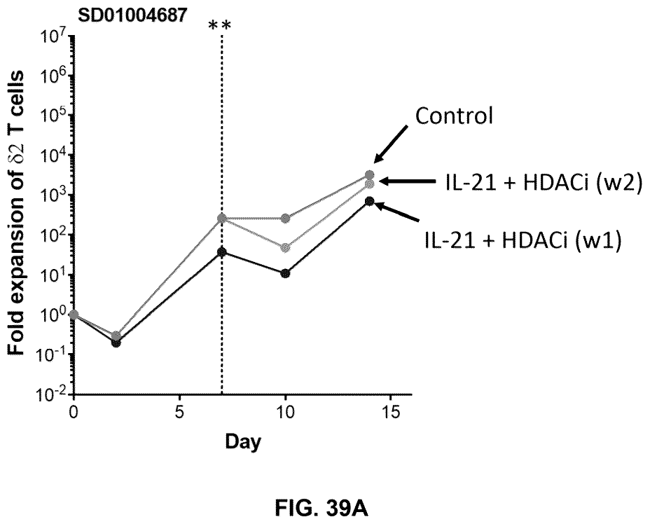

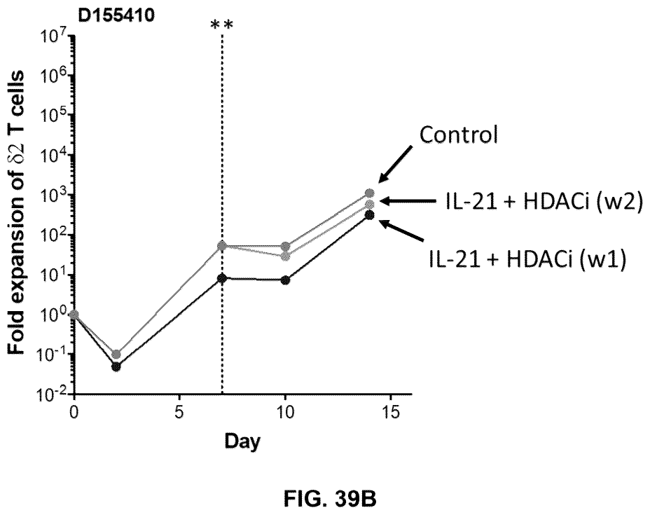

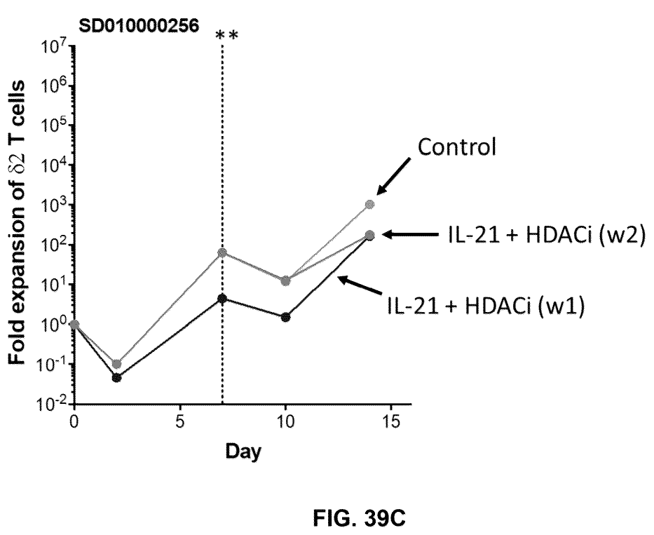

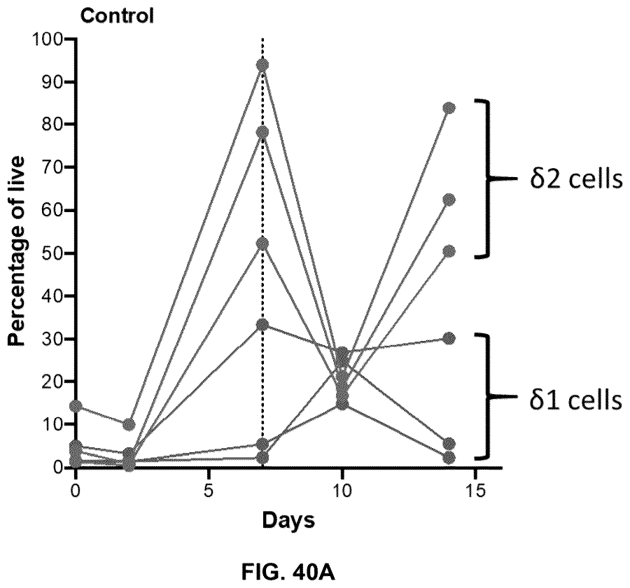

Fold expansion of γδ T cells under Condition 4 (IL-21+HDACi (w2)) and Condition 5 (IL-21+HDACi (w1)) after the first re-stimulation by Process 3 feeder cells (pooled irradiated allogenic PBMCs+LCLs+OKT3) on Day 7 was examined. FIGS. 39A-39C show fold expansion of γδ T cells obtained from 3 different donors (SD01004687 (FIG. 39A), D155410 (FIG. 39B), and SD01000256 (FIG. 39C) treated with control (without IL-21+HDACi), IL-21+HDACi during the first week (w1) (Condition 5), and IL-21+HDACi during the second week (w2) (Condition 4). The results show fold expansion of γδ T cells prepared by IL-21+HDACi during the first week (w1) (Condition 5) is less than that prepared by IL-21+HDACi during the second week (w2) (Condition 4) and the control process. This decrease, however, is recovered on Day 14 after cells expanded in the presence of IL-2+IL-15. (** indicates Process 3 feeder cells re-stimulation)

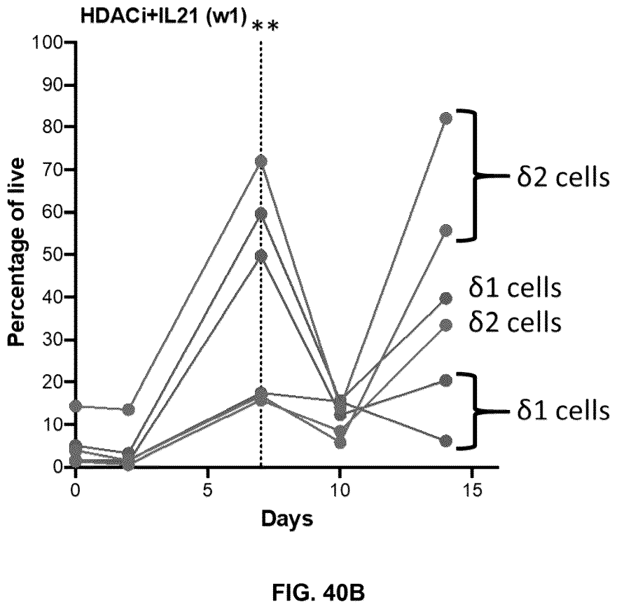

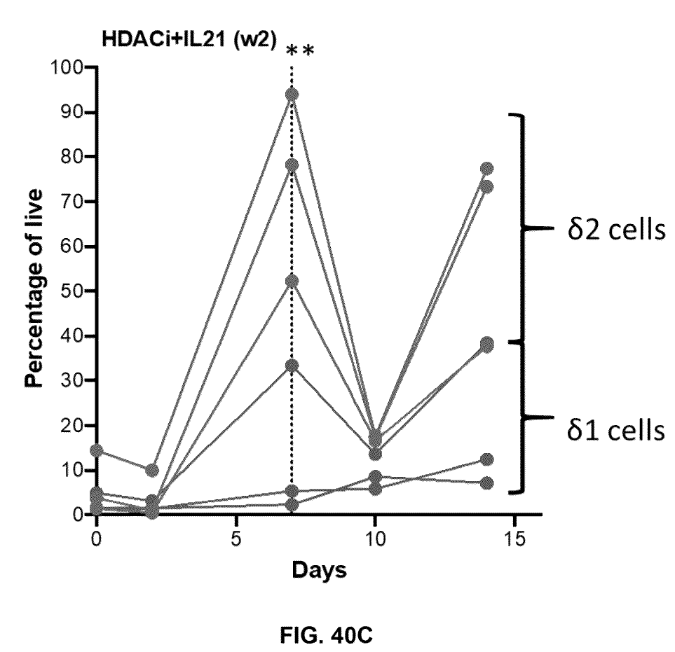

δ2 and δ1 T cells under Condition 4 (IL-21+HDACi (w2)) and Condition 5 (IL-21+HDACi (w1)) after the first re-stimulation by Process 3 feeder cells on Day 7 was examined. FIGS. 40A-40C show % of live δ2 and δ1 T cells treated with control (FIG. 40A), IL-21+HDACi (w1) (FIG. 40B), and IL-21+HDACi (w2) (FIG. 40C). FIG. 40B shows the amount of δ2 T cells decreases during the first week of culture in the presence of HDACi+IL21 (IL-21+HDACi (w1)) as compared with that prepared by the control process (FIG. 40A). FIG. 40C shows the amount of 82 and 81 T cells during the second week of culture in the presence of HDACi+IL21 (IL-21+HDACi (w2)) is comparable to that prepared by the control process (FIG. 40A). (** indicates Process 3 feeder cells re-stimulation)

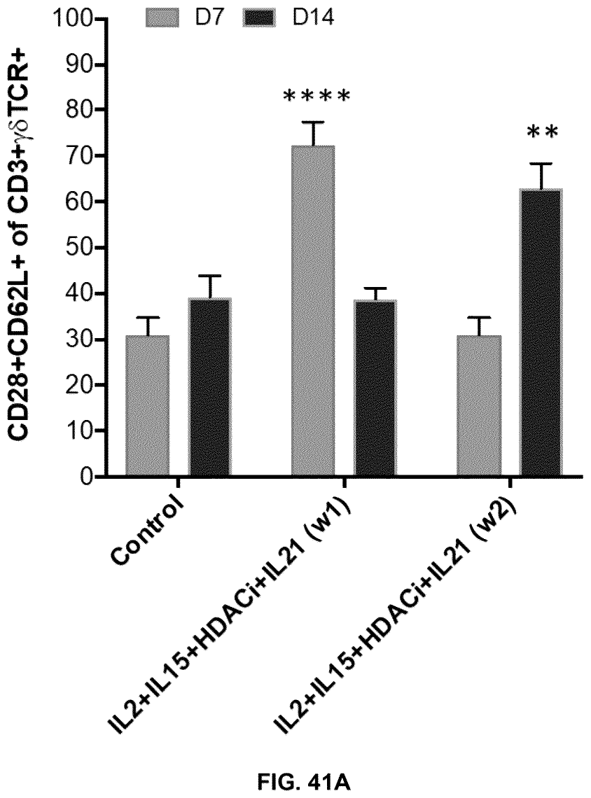

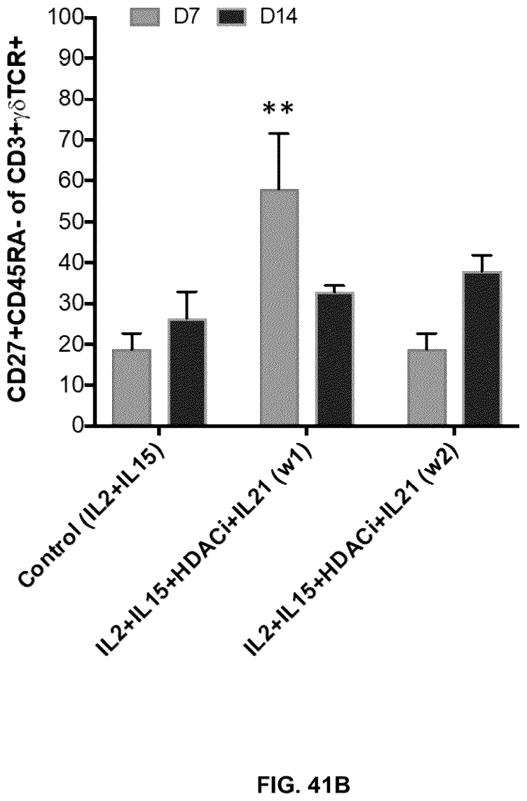

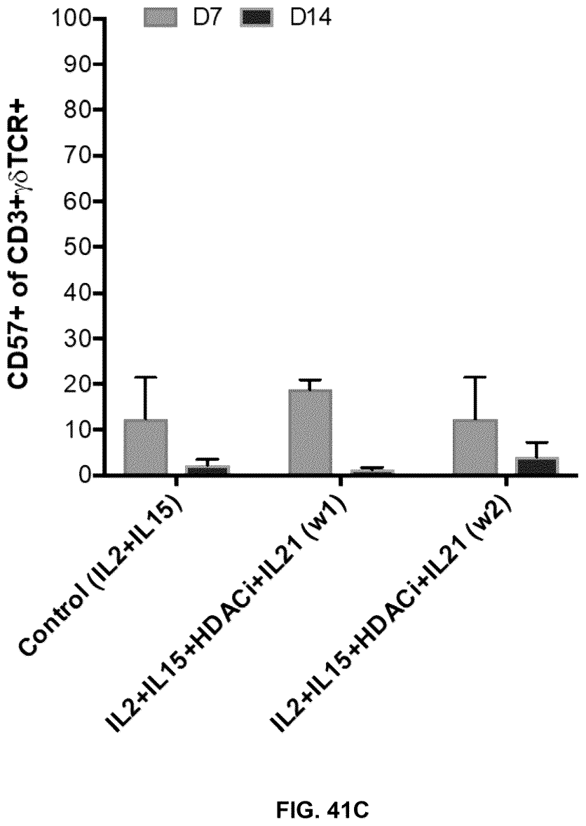

FIG. 41A shows that HDACi+IL-21 during the first week of culture (IL-21+HDACi (w1)) (Condition 5), switch to IL-2+IL-15 during the second week resulted in a decrease of CD28+CD62L+ Tom γδ T cells. On the other hand, IL-2+IL-15 during the first week of culture, switch to IL-21+HDACi (w2) (Condition 4) during the second week resulted in an increase of CD28+CD62L+ Tom γδ T cells. (n=3; mean+SD; ANOVA with multiple comparisons compared to control; ****p<0.0001, **p<0.005)

Similarly, FIG, 41B shows that HDACi+IL-21 during the first week of culture (IL-21+HDACi (w1)) (Condition 5), switch to IL-2+IL-15 during the second week resulted in a decrease of CD27+CD45RA-Tcm γδ T cells. On the other hand, IL-2+IL-15 during the first week of culture, switch to IL-21+HDACi (w2) (Condition 4) during the second week resulted in an increase of CD27+CD45RA-Tcm γδ T cells. (n=3; mean+SD; ANOVA with multiple comparisons compared to control; ****p<0.0001, **p<0.005)

FIG. 41C shows HDACi+IL-21 during the first week of culture (IL-21+HDACi (w1)) (Condition 5) or during the second week of culture (IL-21+HDACi (w2)) (Condition 4) has little effect on CD57+ γδ T cells. (n=3; mean+SD; ANOVA with multiple comparisons compared to control; **p<0.0005; *p<0.5)

In sum, HDACi+IL-21 may promote Tcm in γδ T cells. This Tcm phenotype, however, may be reverted after HDACi+IL-21 removal. In addition, HDACi+IL-21 may affect expansion and δ1 and δ2 T cell subset percentages, if HDACi+IL-21 are used during the first week of culture (Day 0-Day 7).

### Example 9

### Effect of Restimulation in the Presence of IL-12 and IL-18 on γδ T Cell Manufacturing

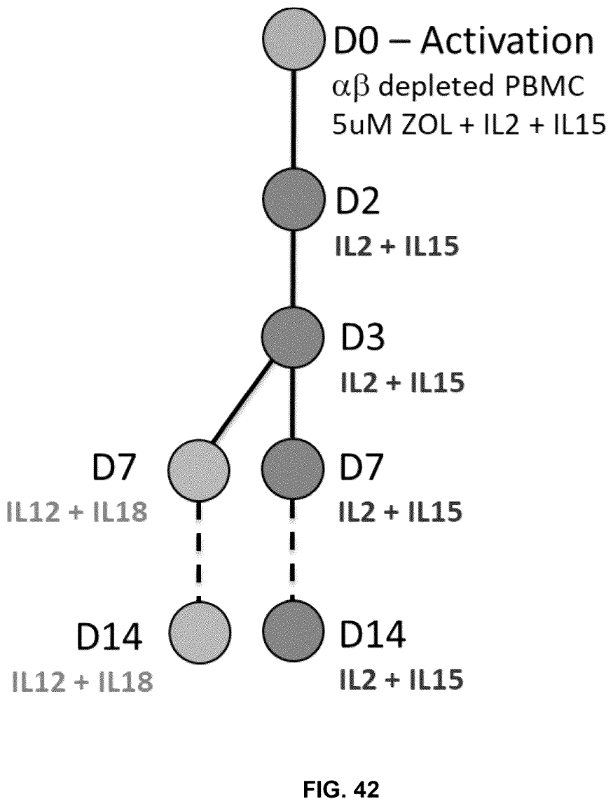

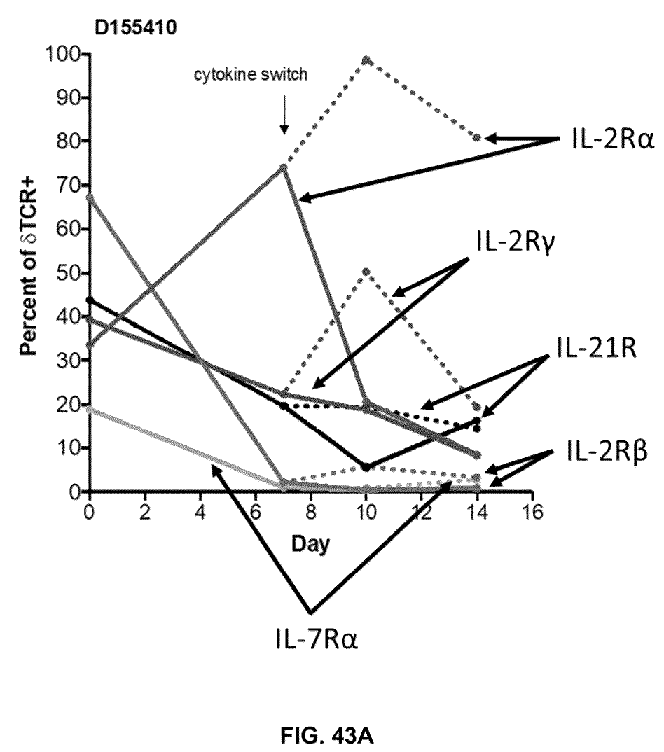

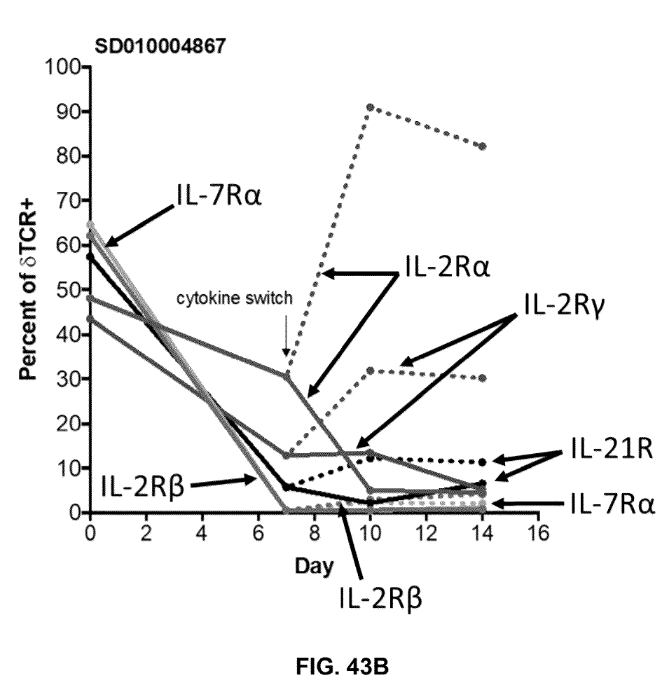

FIG. 42 shows that, on Day 0, PBMCs were depleted of αβTCR-expressing T cells followed by activation in the presence of zoledronate (ZOL) (5 μM), IL-2, and IL-15. Cells were then expanded in the presence of IL-2 and IL-15. On Day 7, cells were either expanded continuously in the presence of IL-2 and IL-15 or expanded in the presence of IL-12 and IL-18 and in the absence of IL-2 and IL-15 from Day 7 to Day 14 (cytokine switch). Cytokine switch decreased expansion of γδ T cells, suggesting that long-term culture with IL-12 and IL-18 may have negative effect on γδ T cell growth. % γδ T cells expressing IL-2 receptors, e.g., IL-2Rα, IL-2Rβ, and IL-2y, IL-7 receptor, e.g., IL-7Rα, and IL-21 receptor (IL-21R) were determined on Day 0, 7, 10, and 14. The results show that cytokine switch from IL-2+IL-15 to IL-12+IL-18 in the absence of IL-2 and IL-15 from Day 7 to Day 14 increases % γδ T cells expressing IL-2Rα, IL-2Ry, and IL-21R in cells obtained from two donors (D155410 (FIG. 43A) and SD010004867 (FIG. 43B)). Dotted lines represent conditions with IL-12+IL-18 (cytokine switch). Cytokine switch has little effect on % γδ T cells expressing IL-2Rβ and IL-7Rα.

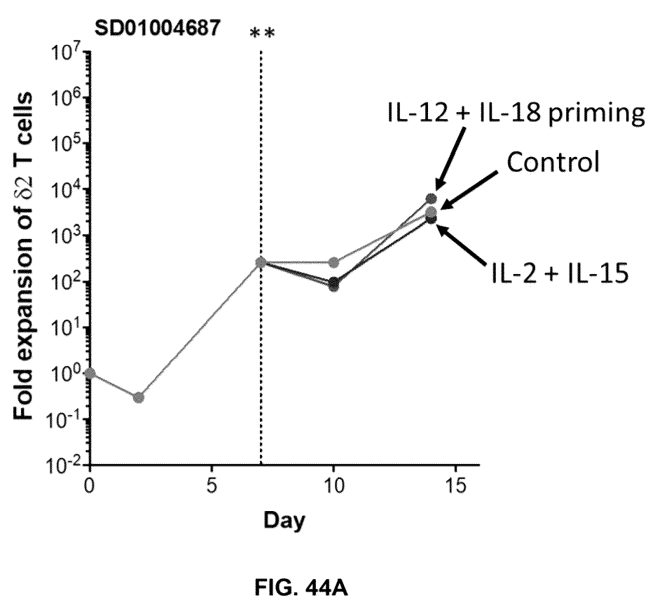

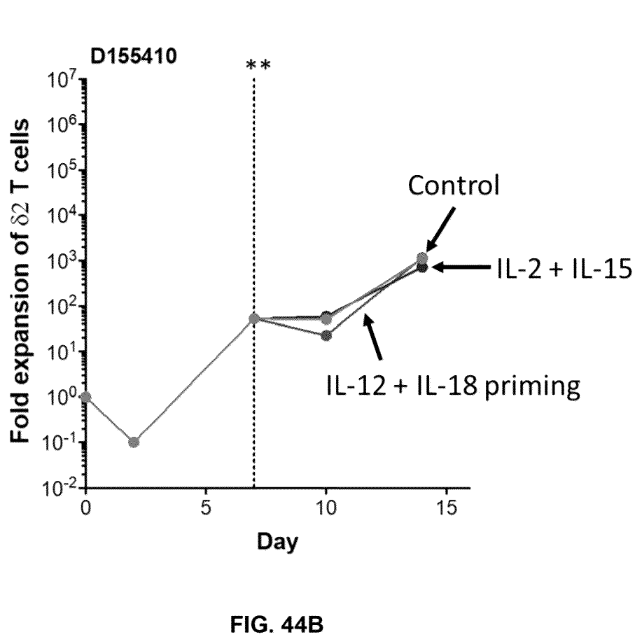

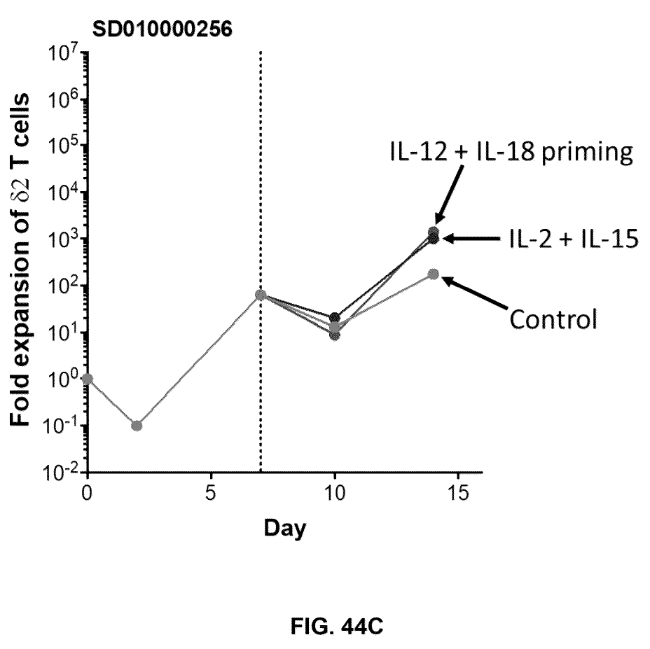

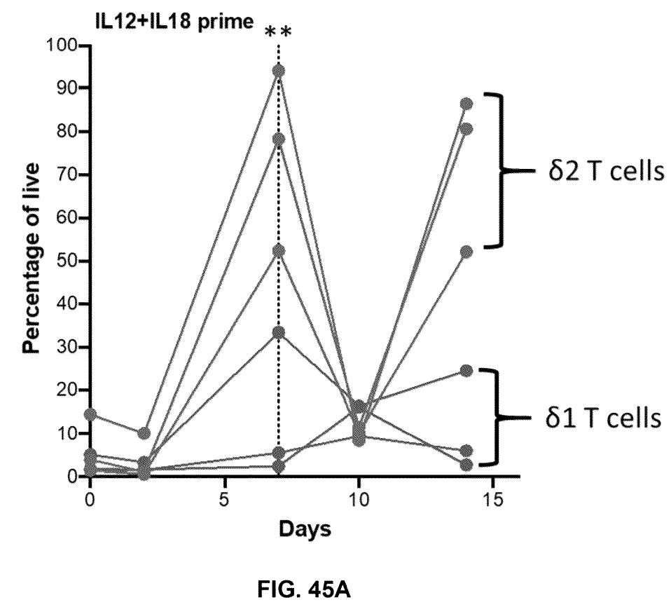

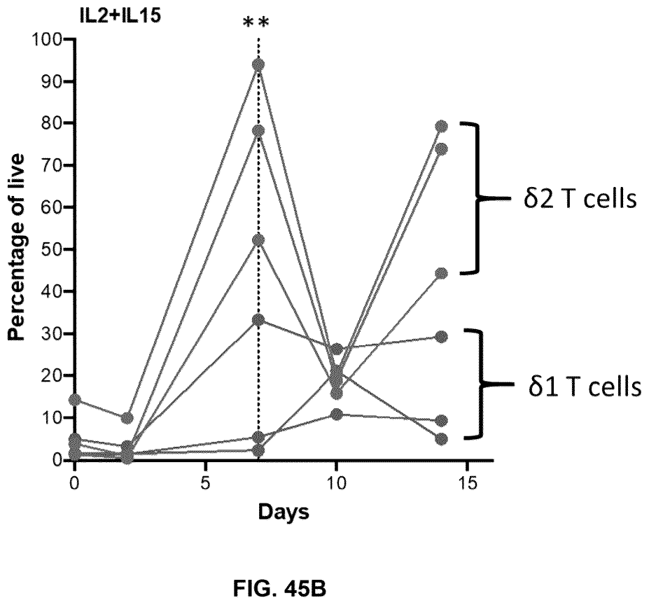

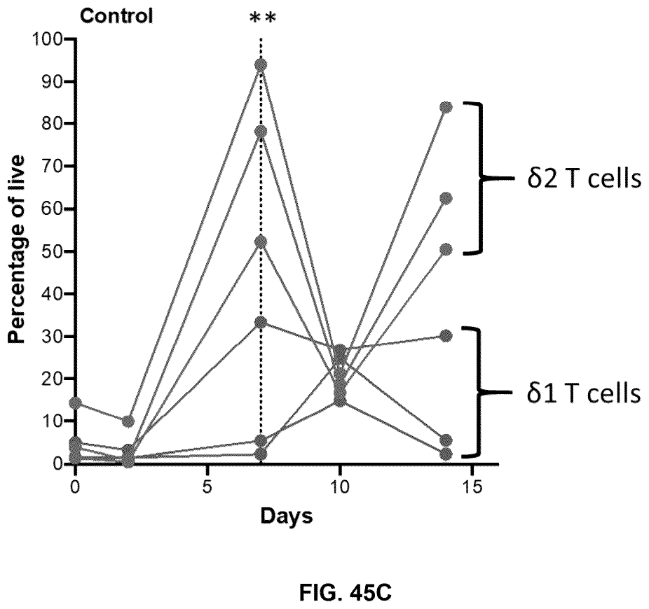

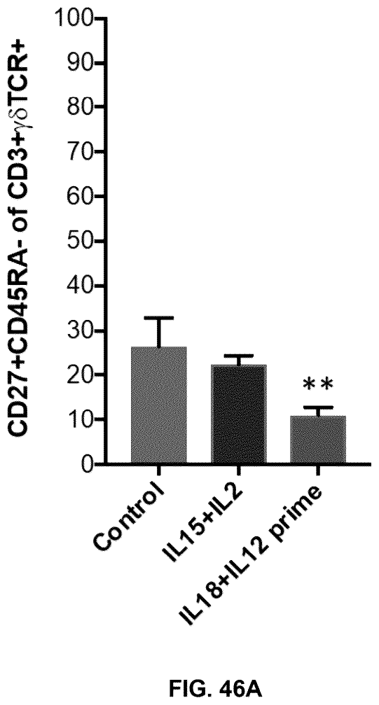

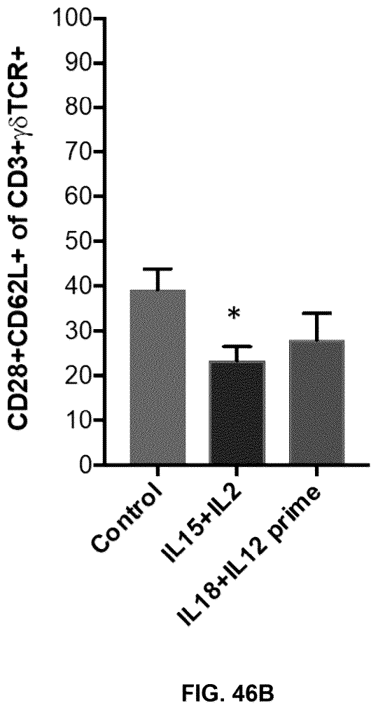

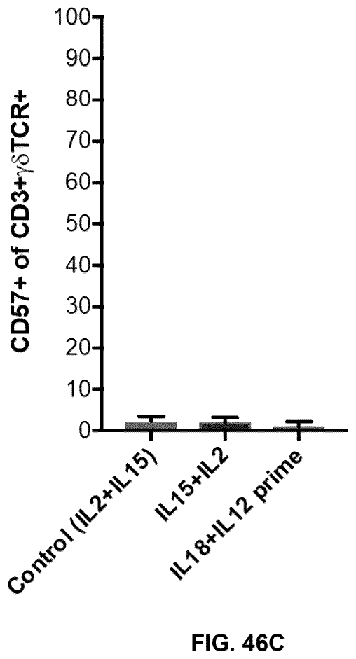

To test the effect on γδ T cell expansion in Condition 3 (IL-12+IL-18 priming on Day 7 re-stimulation) and IL-2+IL-15 after re-stimulation), fold expansion of cells produced by Condition 1 (Control), Condition 2 (IL-2+IL-15) and Condition 3, as shown in FIG. 37, were compared. IL-12+IL-18 priming (Condition 3) has little effect on γδ T cell expansion as compared with that produced by Control and Condition 2 (IL-2+IL-15). There is no significant difference in fold expansion between γδ T cells with IL-12+IL-18 priming and without IL-12+IL-18 priming (IL-2+IL-15) from cells obtained from 3 donors (SD01004687(FIG. 44A), D155410 (FIG. 44B), and SD010000256 (FIG. 44C)). In addition, there is no significant difference between % δ1 T cells and % δ2 T cells prepared with IL-12+IL-18 priming (FIG. 45A) and without IL-12+IL-18 priming (IL-2+IL-15) (FIG. 45B), as compared with Control (FIG. 45C). Phenotype of δ2 T cells prepared by Condition 1 (Control), Condition 2 (IL-2+IL-15), and Condition 3 (IL-12+IL-18 priming), as shown in FIG. 37, were assessed on Day 14 (7 days post IL-12+IL-18 priming), n=3 donors. FIG. 46A shows that Tcm phenotype, e.g., CD27+CD45RA−, of γδ T cells prepared by IL-12+IL-18 priming is significantly reduced as compared with that produced by Control and IL-2+IL-15. FIG. 46B shows that Tcm phenotype, e.g., CD28+CD62L+, of γδ T cells prepared by IL-2+IL-15 is significantly reduced as compared with that produced by Control and IL-12+IL-18 priming. FIG. 46C shows that non-Tcm phenotype, e.g., CD57+, of γδ T cells is minimum in cells produced by Control, IL-2+IL-15, and IL-12+IL-18 priming.

In sum, cytokine switch or IL-12+IL-18 priming may not affect expansion or δ1 and δ2 T cell subset percentages. Cytokine switch or IL-12+IL-18 priming may reduce Tcm γδ T cells by Day 14 as compared with Control method.

### Example 10

### Effect of Initial Stimulation Using Wild Type (WT) K562 Versus K562-41BBL-Mbil15 On γδ T Cell Manufacturing

TABLE 4

| | | Initial | Initial | | |
|---|---|---|---|---|---|
| | | stimulation | Zoledronate | Re-stimulation | Re-stimulation |
| Donor | Process | Feeder | (5 μM) | on Day 7 | on Day 14 |
| D148960 | a | no | Yes | no | no |
| | b | K562 WT | Yes | no | no |
| | c | K562-41BB- | No | no | no |
| | | mbIL15 | | | |
| | d | K562-41BB- | No | K562-41BB- | K562-41BB- |
| | | mbIL15 | | mbIL15 | mbIL15 |
| | e | K562-41BB- | Yes | no | no |
| | | mbIL15 | | | |
| | f | K562-CD86 | Yes | no | no |
| SD01000723 | a | no | Yes | no | no |
| | b | K562 WT | Yes | no | no |
| | c | K562-41BB- | No | no | no |
| | | mbIL15 | | | |
| | d | K562-41BB- | No | K562-41BB- | K562-41BB- |
| | | mbIL15 | | mbIL15 | mbIL15 |
| | e | K562-41BB- | Yes | no | no |
| | | mbIL15 | | | |
| | f | K562-CD86 | Yes | no | no |

γδ T cells obtained from two donors (D148960 and SD01000723) were prepared with initial stimulation using K562 WT, K562-41BB-mbIL15, or K562-CD86 (K562 cell engineered to express CD86) feeder cells according to the processes shown in Table 4.

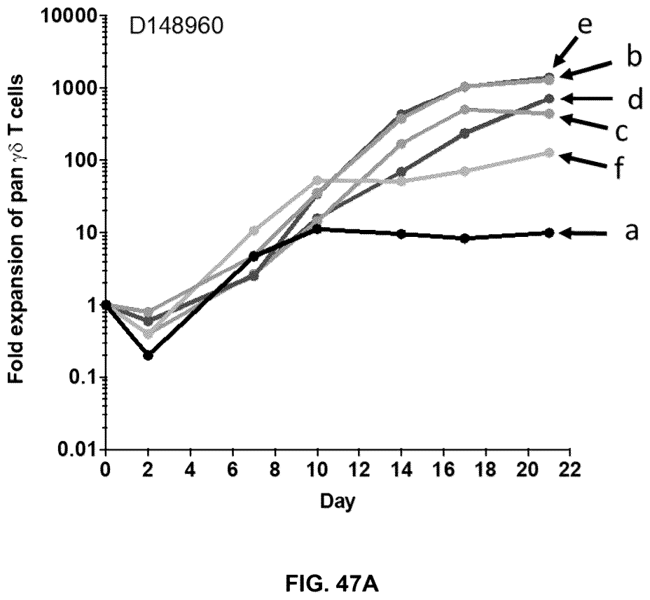

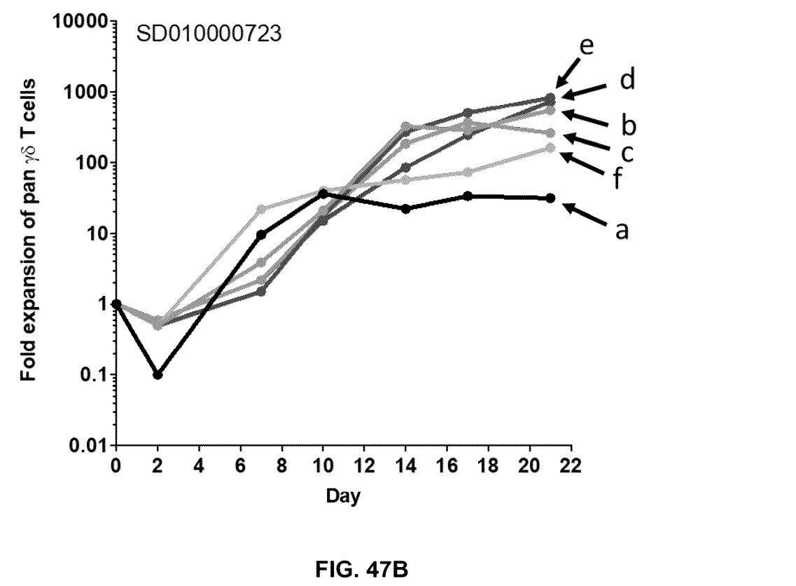

The results show that, in general, fold expansion of pan γδ T cells obtained from two donors (D148960 (FIG. 47A) and SD01000723 (FIG. 47B)) prepared by Processes b-f are higher than that prepared by

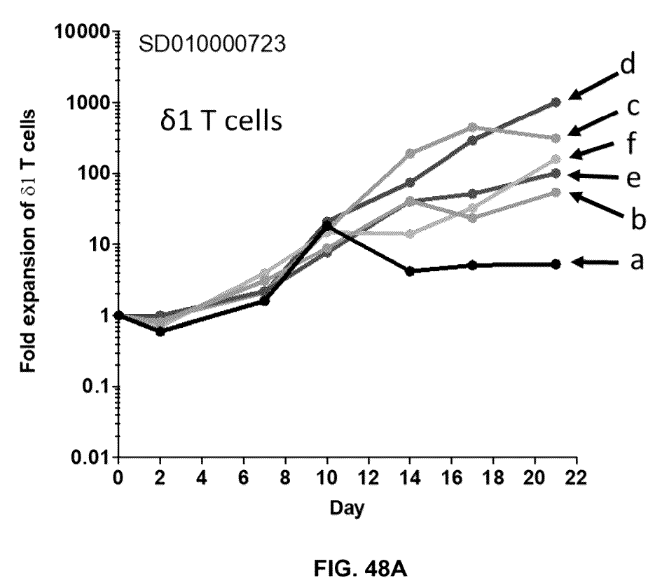

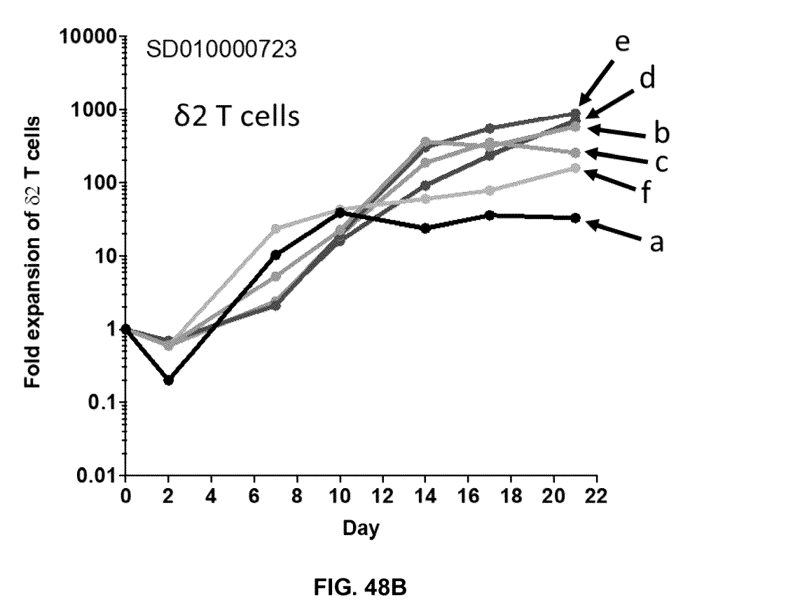

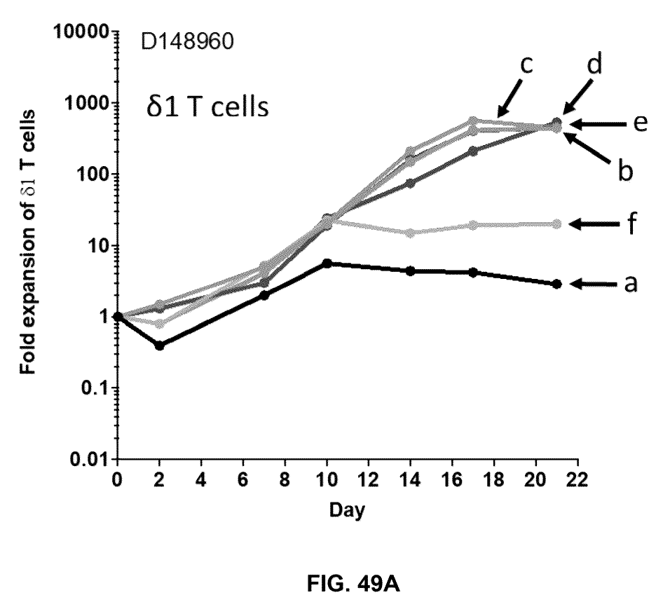

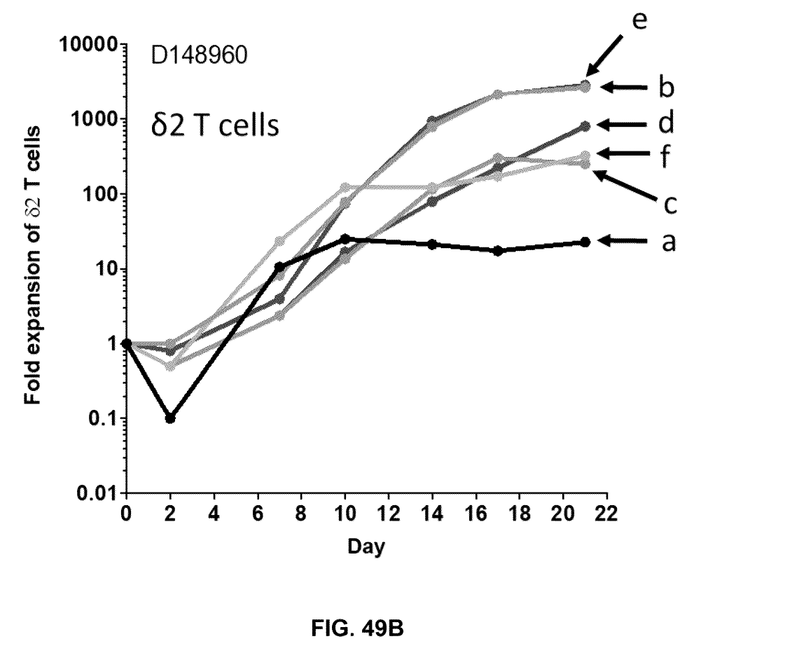

Process a (Control). Initial stimulation with K562 WT (Process b) or K562-41BB-mbIL15 (Processes c, d, and e) yields comparable results. In general, fold expansion of δ1 and δ2 subset T cells obtained from two donors (D148960 (FIGS. 48A and 48B) and SD01000723 (FIGS. 49A and 49B)) prepared by Processes b-f are higher than that prepared by Process a (Control).

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. A method of preparing γδ T cells comprising
isolating γδ T cells from a blood sample of a human subject,
activating the isolated γδ T cells in the presence of a feeder cell and at least one cytokine selected from the group consisting of interleukin (IL)-1, IL-2, IL-12, IL-15, IL-18, and IL-21, interferon (IFN)-α, and IFN-β,
introducing a vector comprising a nucleic acid encoding a T cell receptor (TCR) or a chimeric antigen receptor (CAR) into the activated γδ T cells, and
expanding the introduced γδ T cells.

2. The method of claim 1, wherein the activating is further in the presence of an aminobisphosphonate selected from the group consisting of pamidronic acid, alendronic acid, zoledronic acid, risedronic acid, ibandronic acid, incadronic acid, a salt thereof and a hydrate thereof.

3. The method of claim 1, wherein the isolating comprises contacting the blood sample with anti-α and anti-β T cell receptor (TCR) antibodies and depleting α- and/or β-TCR positive cells from the blood sample.

4. The method of claim 1, wherein the feeder cell is a human cell, a non-human cell, a virus-infected cell, a non-virus infected cell, a cell extract, a particle, a bead, a filament, or a combination thereof.

5. The method of claim 4, wherein the human cell is a K562 cell comprising at least one recombinant protein is selected from the group consisting of CD86, 4-1BBL, IL-15, and any combination thereof.

6. The method of claim 1, further comprising restimulating the expanded γδ T cells in the presence of a feeder cell.

7. An in vitro method of expanding γδ T cells comprising
isolating γδ T cells from a blood sample of a human subject,
activating the isolated γδ T cells in the presence of at least one cytokine selected from the group consisting of interleukin (IL)-1, IL-2, IL-12, IL-15, IL-18, IL-21, interferon (IFN)-α, and IFN-β and an aminobisphosphonate in the absence of a feeder cell,
expanding the activated γδ T cells, and
restimulating the expanded γδ T cells in the presence of a feeder cell.

8. The method of claim 7, wherein the aminobisphosphonate is selected from the group consisting of pamidronic acid, alendronic acid, zoledronic acid, risedronic acid, ibandronic acid, incadronic acid, a salt thereof and a hydrate thereof.

9. The method of claim 7, wherein the isolating comprises contacting the blood sample with anti-α and anti-β T cell receptor (TCR) antibodies and depleting α- and/or β-TCR positive cells from the blood sample.

10. The method of claim 7, wherein the feeder cell is a human cell, a non-human cell, a virus-infected cell, a non-virus infected cell, a cell extract, a particle, a bead, a filament, or a combination thereof.

11. The method of claim 7, wherein the expanding is in the absence of an aminobisphosphonate and in the presence of at least one cytokine.

12. The method of claim 7, wherein the feeder cell comprises peripheral blood mononuclear cells (PBMCs), monocytes, and/or lymphoblastoid cells (LCLs).

13. The method of claim 7, wherein the restimulating is performed in the presence of OKT3.

14. The method of claim 7, further comprising introducing a vector comprising a nucleic acid encoding a T cell receptor (TCR) or a chimeric antigen receptor (CAR) into the activated γδ T cells before the expanding.

15. The method of claim 14, wherein the vector comprises a nucleic acid encoding a TCR and a nucleic acid encoding CD8αβ or CD8α.

16. The method of claim 7, wherein the restimulating is in the presence of at least one cytokine.

17. The method of claim 7, wherein the restimulating is in the absence of a cytokine.

18. The method of claim 12, wherein the feeder cell is pulsed with an aminobisphosphonate selected from the group consisting of pamidronic acid, alendronic acid, zoledronic acid, risedronic acid, ibandronic acid, incadronic acid, a salt thereof and a hydrate thereof.

19. The method of claim 7, wherein the restimulating is performed on Day 7 and/or Day 14 after the activating performed on Day 0.

20. A method of treating an autoimmune disease, comprising administering to a patient in need thereof an effective amount of T cells prepared by
isolating γδ T cells from a blood sample of a human subject,
activating the isolated γδ T cells in the presence of a feeder cell and at least one cytokine selected from the group consisting of interleukin (IL)-1, IL-2, IL-12, IL-15, IL-18, and IL-21, interferon (IFN)-α, and IFN-β,
introducing a vector comprising a nucleic acid encoding a T cell receptor (TCR) or a chimeric antigen receptor (CAR) into the activated γδ T cells, and
expanding the introduced γδ T cells,
wherein the autoimmune disease is selected from the group consisting of Arthritis, Chronic obstructive pulmonary disease, Ankylosing Spondylitis, Crohn's Disease (one of two types of idiopathic inflammatory bowel disease “IBD”), Dermatomyositis, Diabetes mellitus type 1, Endometriosis, Goodpasture's syndrome, Graves' disease, Guillain-Barre syndrome (GBS), Hashimoto's disease, Hidradenitis suppurativa, Kawasaki disease, IgA nephropathy, Idiopathic thrombocytopenia purpura, Interstitial cystitis, Lupus erythematosus, Mixed Connective Tissue Disease, Morphea, Myasthenia gravis, Narcolepsy, Neuromyotonia, Pemphigus vulgaris, Pernicious anaemia, Psoriasis, Psoriatic Arthritis, Polymyositis, Primary biliary cirrhosis, Relapsing polychondritis, Rheumatoid arthritis, Schizophrenia, Scleroderma, Sjogren's syndrome, Stiff person syndrome, Temporal arteritis (also known as “giant cell arteritis”), Ulcerative Colitis (one of two types of idiopathic inflammatory bowel disease “IBD”), Vasculitis, Vitiligo, and Wegener's granulomatosis.
